SEQUENCE LISTING (265 sequences)

SEQ ID NO: 1 - protein, 324 aa, Crotalus atrox
MNSLVALVLLGQIIGSTLSFQLAGNMDCNTKGTKDWADIGVRYINEHKLHGYKQALNIIKIFRLLPSDGRSVIFHFKLNLLETECHVLDPTPLENCTVRQQNNHAVEMDCNVRIIHDIATFEDEVFVKCNSTPDSVENVRRNCPKCPILLPPNDPHVIDSVEYVLNKHNEKLSGHIYEVLEISRGQHKYEPEAFYVEFAIVEVNCTAEEAHDDHHDCHPNTDGENHIGFCRATVFRSHASLEKPKEEMFESDCVILHVKKGHAYSHLIEHHVGKYNTSPGRNSTDECVVQCPVAFVNKEVPKDISDHDTPPVEGCPGKALHFQL

SEQ ID NO: 2 - protein, 324 aa, Gloydius blomhoffii
MHFLVALVLLGQIIGSTLSSQVRGDLECNDREAKEWADQAVRYINEHKLHEYKQALNVIKNIVVVPWNGDLVAVFLKLNLLETECHVLDPTPVEKCTIRPQQNHAVEMDCDAKIMFDVETFKQDVFVKCHSTPDSVEDVRRNCLKCPILLSPSDPHVVDSVEYVLNKHNEQLSGHVYEVLEISRGQHKYEPEAFYVEFAIVEVNCTAQEAHDDHHHCHPNTAGEDHIAFCKATVFRSHASLEKPKHENFESDCVILDVKEGHAHSHLIEHHIGKYSTSPGQNSTVECVAECPVAFVNKEVPTDISDRHTTPVKGCPGKILHFQL

SEQ ID NO: 3 - protein, 341 aa, Protobothrops flavoviridis
MNSLVALVLLGQIIGSTLSSQVRGDLECDDKEAKNWADDAVRYINEHKLHGHKQALNVIKNICVVPWNGDLVAVFLELNLLETECHVLDPTPVEKCTVRQQHNHAVEMDCDAKIMFNVETFKRDVFVKCHSTPDSVENVRRNCSKCPILLPPNNPHVVDSVEYVLNKHNEKLSGHIYEVLEISRGQHKYEPEAYYLEFVIVEINCTAQEAHDDHHQCHPYTAGEDHIAFCRSTVFRSHASLEKPKDEKFESDCVILDVKDGHAHSHLIQQHIEKNSISPEHNITILNFVHPDNHTSTSHESHEHVAEVPVVFVKKELPTDISDHHTTPVKGCPGKVHHFEL

SEQ ID NO: 4 - protein, 341 aa, Bothrops jararaca
MNSLVALVLLGQIIGSTLSSQVRGDLECDEKDAKEWTDTGVRYINEHKLHGYKYALNVIKNIVVVPWDGDWVAVFLKLNLLETECHVLDPTPVKNCTVRPQHNHAVEMDCDVKIMFNVDTFKEDVFAKCHSTPDSVENVRRNCPKCPILLPSNNPQVVDSVEYVLNKHNEKLSDHVYEVLEISRGQHKYEPEAYYVEFAIVEVNCTAQELHDDHHHCHPNTAGEDHIAFCRATVFRSHASLEKPKDEQFESDCVILHVKEGHAHSHLIQQHVEKDSISPEHNNTALNFVHPHNDTSTSHESHEHLAEVPVAFVKKELPKDISDRHTTPVKGCPGKVHHFEL

SEQ ID NO: 5 - protein, 344 aa, Crotalus atrox
MNSLVALVLLGQIIGSTHSYMVREEDLNCNSKAAKYRADQAVRYINEHNLHGYKQALNIIKTIRLLPSDGLLVVIYLKLNLLETECHVLDPTPVENCTVRQQNNHAVETDCDVKVIYNIETFKDEFLVKCHSTPGSVEKILQDCPKCPILLSLSDPHVVDSVEYVLNKHNEQLSGHVYEVLEISRGQHKYEPEAFYVEFAIVEVNCTAQEAHDDHHHCHPNTAGENHIGFCRATVFRSHASLEKPKDEMSESDCVILDVKEGHAHSHLIEHHTGKHSTSPGHNSTVLNLAHSHNHTSASHESHSHEHVMEVPVAVANREDPTDIPHHHTPSVDGCPGKALHFQL

SEQ ID NO: 6 - protein, 343 aa, Crotalus atrox
MNSLVALVLLGQIIGSTLSYMVKEEDMNCNSKAAKYRADQAVRYINEHKLHGYKQALNIIKTFHLLPPERFTTVFYLKLNLLETECHVLDPTPVENCTVRPQNNHAVEMDCDVKINFNFEFFKTEIFVKCNSTPDSVENVQRNCPKCPILLPPNDPHVINSVEYMLNKHNEKLSGHVYEVLEISRGQHKYEPEAFYVEFAIVEVNCTAEEAHDDHHHCRPNTAGEDHIGFCRSTVFRSHASLEKPKDEMFESDCVILHVKKGHSHSHLIQQHIGKYNISPGHHNTVLNLAHSHNDTRASQESHSDECVVECPVAFVKKEVPTHIPHHTHPVEGCPGKVHHFKL

SEQ ID NO: 7 - protein, 324 aa, Crotalus atrox
MNSLVALVLLGQIIGSTLSSQVRGDLECDDKEAKEWADMAVRYINEHKLHGYKQALNVIKNILVVPWDGDLVAVYLKLNLLETECHVLDPHPVENCTIRPQNNHAVKMDCNAKIMFDVVTFKQDVFVKCHSTPDSVENIRRNCPKCPILLPRNDSHVVDSVEYVLNKHNEKLSGHIYEVLEISRGQHKYEPEAFYVEFAIVEVNCTAQEAHDDHHHCHPNTAGEDHIGFCRATVFRSHASLEKPKDEKFESDCVILHVKEGHVHSHLIEHHTGKYSTSPGSNSTDECVVQCPVAFVNKELPTDISDHHTTPVKGCPGKVFHFQL

SEQ ID NO: 8 - protein, 342 aa, Crotalus atrox
MNSLVALVLLGQIIGCTFSHHLQSQIDCNGEDAEKWADMAVHYINEHNLHGYKYTTNVINEIHVLPWRPHGEIIFLELKVLETQCHVLDPTPVENCTVRPLHYHAVGGDCDVKIIHDEGVDKVIGAKCHSDPDSVENVRQNCPECPILLSLSDPHVVDCVEYVLNKYNEKLSGHIYEVLEISRGQHKYEPEAFYVEFAIVEVNCTAEEAHDDHHHCHPNTAGENHIGFCRATVFRSHASLEKPKDEMFESDCVIFDVKEGHAHSHLIEHHTGKYSTSPGHNNTVLNLIHSHNHTSASHESHSDEHMTEVPVAVAKREVPTDVPHDHTHPVKLCPGKVHHFKL

SEQ ID NO: 9 - DNA, 24 bp, synthetic construct
  source 1..24: mol_type = other DNA
atgaattccctggtagctctcgtg

SEQ ID NO: 10 - DNA, 25 bp, synthetic construct
  source 1..25: mol_type = other DNA
gagtcttccttctttccaactagtg

SEQ ID NO: 11 - DNA, 25 bp, synthetic construct
  source 1..25: mol_type = other DNA
taaaataccgtcgtaaacctctccg

SEQ ID NO: 12 - DNA, 20 bp, synthetic construct
  source 1..20: mol_type = other DNA
ccggagaggtttacgacggt

SEQ ID NO: 13 - DNA, 25 bp, synthetic construct
  source 1..25: mol_type = other DNA
tctcaatgctctctagtatggctgg

SEQ ID NO: 14 - DNA, 24 bp, synthetic construct
  source 1..24: mol_type = other DNA
tttaaggtagactgccaccaaatc

SEQ ID NO: 15 - DNA, 27 bp, synthetic construct
  source 1..27: mol_type = other DNA
tctcaagtgaggggggatttagaatgt

SEQ ID NO: 16 - DNA, 28 bp, synthetic construct
  source 1..28: mol_type = other DNA
gtttgctattgtggaggttaactgcact

SEQ ID NO: 17 - DNA, 26 bp, synthetic construct
  source 1..26: mol_type = other DNA
caccacacattcatcagtactgttgc

SEQ ID NO: 18 - protein, 17 aa, synthetic construct
VTTGSQCAEGLCCDQCK

SEQ ID NO: 19 - protein, 30 aa, synthetic construct
RHDNAQLLTSIDFDGPTIGLAYIGSICDPK

SEQ ID NO: 20 - protein, 31 aa, synthetic construct
RHDNAQLLTSIDFDGPTIGLAYIGSICDPKR

SEQ ID NO: 21 - protein, 15 aa, synthetic construct
RSTGVVQDHSEINLR

SEQ ID NO: 22 - protein, 14 aa, synthetic construct
STGVVQDHSEINLR

SEQ ID NO: 23 - protein, 24 aa, synthetic construct
YFSDCSYIQCWNFIMNQKPQCILK

SEQ ID NO: 24 - protein, 15 aa, synthetic construct
YVELFIVVDHGMFTK

SEQ ID NO: 25 - protein, 8 aa, synthetic construct
DGICDPKR

SEQ ID NO: 26 - protein, 16 aa, synthetic construct
ISHDNAQLLTSTDFNR

SEQ ID NO: 27 - protein, 8 aa, synthetic construct
QAIGLAYR

SEQ ID NO: 28 - protein, 15 aa, synthetic construct
QAIGLAYRDGICDPK

SEQ ID NO: 29 - protein, 11 aa, synthetic construct
RSTGVVQDHSK

SEQ ID NO: 30 - protein, 10 aa, synthetic construct
STGVVQDHSK

SEQ ID NO: 31 - protein, 20 aa, synthetic construct
VSMVDRNDDTCTGQSADCPR

SEQ ID NO: 32 - protein, 24 aa, synthetic construct
YFSDCSYIQCWDFIMNQNPQCILK

SEQ ID NO: 33 - protein, 15 aa, synthetic construct
YVELFIVVDHGMYTK

SEQ ID NO: 34 - protein, 16 aa, synthetic construct
AWVYEIFNTINEIFQR

SEQ ID NO: 35 - protein, 30 aa, synthetic construct
SHDNAQLLTSIDLDGPTIGLAYIGGICDPK

SEQ ID NO: 36 - protein, 16 aa, synthetic construct
AWVYEIVNTINEIFQR

SEQ ID NO: 37 - protein, 18 aa, synthetic construct
IIVQSSADITLDLFGTWR

SEQ ID NO: 38 - protein, 10 aa, synthetic construct
NLCCDAATCK

SEQ ID NO: 39 - protein, 30 aa, synthetic construct
SHDNAQLLTSINFDGQTIGWAYIGGICDSK

SEQ ID NO: 40 - protein, 31 aa, synthetic construct
SHDNAQLLTSINFDGQTIGWAYIGGICDSKR

SEQ ID NO: 41 - protein, 55 aa, synthetic construct
STGVVQDFSPINFLVAITMAHEMGHNLGMTHDENYCSCGGFACIMSPVISPQPSK

SEQ ID NO: 42 - protein, 10 aa, synthetic construct
AYTGSMCDPR

SEQ ID NO: 43 - protein, 11 aa, synthetic construct
AYTGSMCDPRK

SEQ ID NO: 44 - protein, 21 aa, synthetic construct
ISHDNAQLLTAINFQENIIGR

SEQ ID NO: 45 - protein, 31 aa, synthetic construct
ISHDNAQLLTAINFQENIIGRAYTGSMCDPR

SEQ ID NO: 46 - protein, 15 aa, synthetic construct
IWVHEIVNTINVFYR

SEQ ID NO: 47 - protein, 11 aa, synthetic construct
YIELVIVADHR

SEQ ID NO: 48 - protein, 9 aa, synthetic construct
YNSNLNTIR

SEQ ID NO: 49 - protein, 24 aa, synthetic construct
YNSNLNTIRIWVHEIVNTINVFYR

SEQ ID NO: 50 - protein, 15 aa, synthetic construct
AAKDECDMADVCTGR

SEQ ID NO: 51 - protein, 14 aa, synthetic construct
ACSNGQCVDVTTPY

SEQ ID NO: 52 - protein, 28 aa, synthetic construct
CPIMADQCIALFGPGATVSQDACFQFNR

SEQ ID NO: 53 - protein, 12 aa, synthetic construct
DECDMADVCTGR

SEQ ID NO: 54 - protein, 8 aa, synthetic construct
DHREFLIK

SEQ ID NO: 55 - protein, 30 aa, synthetic construct
DTCTCGTRPCVMAGALSCEASFLFSDCSQK

SEQ ID NO: 56 - protein, 9 aa, synthetic construct
EGNHYGYCR

SEQ ID NO: 57 - protein, 9 aa, synthetic construct
IACEPQDVK

SEQ ID NO: 58 - protein, 17 aa, synthetic construct
LRQGAQCAEGLCCDQCR

SEQ ID NO: 59 - protein, 14 aa, synthetic construct
MYDIVNVITPIYHR

SEQ ID NO: 60 - protein, 8 aa, synthetic construct
NMPQCILK

SEQ ID NO: 61 - protein, 10 aa, synthetic construct
NNNGYCYNGK

SEQ ID NO: 62 - protein, 14 aa, synthetic construct
NPCNIYYSPNDEDK

SEQ ID NO: 63 - protein, 15 aa, synthetic construct
QGAQCAEGLCCDQCR

SEQ ID NO: 64 - protein, 41 aa, synthetic construct
SHDNAQLLTGINFNGPTAGLGYLGGICNTMYSAGIVQDHSK

SEQ ID NO: 65 - protein, 13 aa, synthetic construct
TCRDPCCDATTCK

SEQ ID NO: 66 - protein, 26 aa, synthetic construct
TDVVSPAVCGNYFVEVGEECDCGSPR

SEQ ID NO: 67 - protein, 17 aa, synthetic construct
LHSWVECESGECCDQCR

SEQ ID NO: 68 - protein, 12 aa, synthetic construct
AMVTKNNGDLDK

SEQ ID NO: 69 - protein, 26 aa, synthetic construct
ASMSECDPAEHCTGQSSECPADVFHK

SEQ ID NO: 70 - protein, 11 aa, synthetic construct
DNSPGQNNPCK

SEQ ID NO: 71 - protein, 14 aa, synthetic construct
ENGNKIPCAPEDVK

SEQ ID NO: 72 - protein, 10 aa, synthetic construct
FVELFLVVDK

SEQ ID NO: 73 - protein, 8 aa, synthetic construct
GNYYGYCR

SEQ ID NO: 74 - protein, 9 aa, synthetic construct
GNYYGYCRK

SEQ ID NO: 75 - protein, 14 aa, synthetic construct
HDNAQLLTAIDLDR

SEQ ID NO: 76 - protein, 28 aa, synthetic construct
HDNAQLLTAIDLDRVIGLAYVGSMCHPK

SEQ ID NO: 77 - protein, 29 aa, synthetic construct
HDNAQLLTAIDLDRVIGLAYVGSMCHPKR

SEQ ID NO: 78 - protein, 18 aa, synthetic construct
ITVKPEAGYTLNAFGEWR

SEQ ID NO: 79 - protein, 19 aa, synthetic construct
ITVKPEAGYTLNAFGEWRK

SEQ ID NO: 80 - protein, 25 aa, synthetic construct
ITVKPEAGYTLNAFGEWRKTDLLTR

SEQ ID NO: 81 - protein, 15 aa, synthetic construct
KENGNKIPCAPEDVK

SEQ ID NO: 82 - protein, 15 aa, synthetic construct
KHDNAQLLTAIDLDR

SEQ ID NO: 83 - protein, 29 aa, synthetic construct
KHDNAQLLTAIDLDRVIGLAYVGSMCHPK

SEQ ID NO: 84 - protein, 16 aa, synthetic construct
KKHDNAQLLTAIDLDR

SEQ ID NO: 85 - protein, 8 aa, synthetic construct
KTDLLTRK

SEQ ID NO: 86 - protein, 15 aa, synthetic construct
LYCKDNSPGQNNPCK

SEQ ID NO: 87 - protein, 10 aa, synthetic construct
MFYSNEDEHK

SEQ ID NO: 88 - protein, 13 aa, synthetic construct
MYEIVNTVNEIYR

SEQ ID NO: 89 - protein, 32 aa, synthetic construct
MYEIVNTVNEIYRYMYIHVALVGLEIWSNEDK

SEQ ID NO: 90 - protein, 50 aa, synthetic construct
MYEIVNTVNEIYRYMYIHVALVGLEIWSNEDKITVKPEAGYTLNAFGEWR

SEQ ID NO: 91 - protein, 42 aa, synthetic construct
NGQPCLDNYGYCYNGNCPIMYHQCYDLFGADVYEAEDSCFER

SEQ ID NO: 92 - protein, 11 aa, synthetic construct
NQKGNYYGYCR

SEQ ID NO: 93 - protein, 15 aa, synthetic construct
TRMYEIVNTVNEIYR

SEQ ID NO: 94 - protein, 34 aa, synthetic construct
TRMYEIVNTVNEIYRYMYIHVALVGLEIWSNEDK

SEQ ID NO: 95 - protein, 14 aa, synthetic construct
VCSNGHCVDVATAY

SEQ ID NO: 96 - protein, 14 aa, synthetic construct
VIGLAYVGSMCHPK

SEQ ID NO: 97 - protein, 15 aa, synthetic construct
VIGLAYVGSMCHPKR

SEQ ID NO: 98 - protein, 19 aa, synthetic construct
YMYIHVALVGLEIWSNEDK

SEQ ID NO: 99 - protein, 37 aa, synthetic construct
YMYIHVALVGLEIWSNEDKITVKPEAGYTLNAFGEWR

SEQ ID NO: 100 - protein, 20 aa, synthetic construct
YNPFRFVELFLVVDKAMVTK

SEQ ID NO: 101 - protein, 10 aa, synthetic construct
AFDSNEDDHK

SEQ ID NO: 102 - protein, 10 aa, synthetic construct
AHVASMCEPK

SEQ ID NO: 103 - protein, 8 aa, synthetic construct
GDDDGYCR

SEQ ID NO: 104 - protein, 9 aa, synthetic construct
GDDDGYCRK

SEQ ID NO: 105 - protein, 19 aa, synthetic construct
HDNAQLLTAIDFSGSTIGK

SEQ ID NO: 106 - protein, 24 aa, synthetic construct
RSECDIAESCTGQSDDCPTDDFHR

SEQ ID NO: 107 - protein, 23 aa, synthetic construct
SECDIAESCTGQSDDCPTDDFHR

SEQ ID NO: 108 - protein, 14 aa, synthetic construct
VCSNGHCVDVASAY

SEQ ID NO: 109 - protein, 10 aa, synthetic construct
AYIASMCDPK

SEQ ID NO: 110 - protein, 14 aa, synthetic construct
FCTNGHCVDVATAY

SEQ ID NO: 111 - protein, 9 aa, synthetic construct
GDDYGYCRK

SEQ ID NO: 112 - protein, 15 aa, synthetic construct
LYCNDNSPGQNNPCK

SEQ ID NO: 113 - protein, 24 aa, synthetic construct
RSECDIAESCTGQSADCPMDDFHK

SEQ ID NO: 114 - protein, 11 aa, synthetic construct
LFCNVNDFLCR

SEQ ID NO: 115 - protein, 12 aa, synthetic construct
NRQCVDVTTAYK

SEQ ID NO: 116 - protein, 10 aa, synthetic construct
QCVDVTTAYK

SEQ ID NO: 117 - protein, 12 aa, synthetic construct
RYVELVIVADHR

SEQ ID NO: 118 - protein, 11 aa, synthetic construct
YVELVIVADHR

SEQ ID NO: 119 - protein, 17 aa, synthetic construct
EQPSYEFSDCSQNQYLR

SEQ ID NO: 120 - protein, 15 aa, synthetic construct
YSADPDYGMVNHGTK

SEQ ID NO: 121 - protein, 11 aa, synthetic construct
LFCEVNNFPCR

SEQ ID NO: 122 - protein, 14 aa, synthetic construct
ENGVNIPCSYEDVK

SEQ ID NO: 123 - protein, 14 aa, synthetic construct
FALVGLEIWSNGDK

SEQ ID NO: 124 - protein, 25 aa, synthetic construct
ITVQSSAYNTLDSFEEWRETDLLTR

SEQ ID NO: 125 - protein, 13 aa, synthetic construct
LFCEFNNFPCQYK

SEQ ID NO: 126 - protein, 16 aa, synthetic construct
RHDNAQLLTAIDFDGR

SEQ ID NO: 127 - protein, 23 aa, synthetic construct
SECDIAESCTGQSADCPTDDFHR

SEQ ID NO: 128 - protein, 14 aa, synthetic construct
TLGLASVSSMCNQK

SEQ ID NO: 129 - protein, 15 aa, synthetic construct
YSEDLDYGMVDHGTK

SEQ ID NO: 130 - protein, 12 aa, synthetic construct
DDCDMAESCTGR

SEQ ID NO: 131 - protein, 20 aa, synthetic construct
DDCDMADLCTGQSAECPTDR

SEQ ID NO: 132 - protein, 12 aa, synthetic construct
DEDIGMVLPGTK

SEQ ID NO: 133 - protein, 8 aa, synthetic construct
GSSYGYCR

SEQ ID NO: 134 - protein, 9 aa, synthetic construct
GSSYGYCRK

SEQ ID NO: 135 - protein, 35 aa, synthetic construct
GSVGVIQEHSTINLLMAVTMAHEMGHNLGMDHDIK

SEQ ID NO: 136 - protein, 9 aa, synthetic construct
IPCPPQDVK

SEQ ID NO: 137 - protein, 23 aa, synthetic construct
IYEIVNTMNEIYIPLNIHVALVR

SEQ ID NO: 138 - protein, 16 aa, synthetic construct
KYVEFVVVLDHGMYTK

SEQ ID NO: 139 - protein, 11 aa, synthetic construct
LFCFPNKPGEK

SEQ ID NO: 140 - protein, 17 aa, synthetic construct
LTPGSQCADGVCCDQCR

SEQ ID NO: 141 - protein, 9 aa, synthetic construct
NGHPCLNNK

SEQ ID NO: 142 - protein, 15 aa, synthetic construct
NQCISFFGPSATVAK

SEQ ID NO: 143 - protein, 10 aa, synthetic construct
NVCNVIYMSR

SEQ ID NO: 144 - protein, 22 aa, synthetic construct
NVCNVIYMSRDEDIGMVLPGTK

SEQ ID NO: 145 - protein, 11 aa, synthetic construct
RPQCILNEPLR

SEQ ID NO: 146 - protein, 50 aa, synthetic construct
RPQCILNEPLRTDIVSPPVCGNELLEVGEECDCGSPGNCQNPCCNATTCK

SEQ ID NO: 147 - protein, 24 aa, synthetic construct
SHDNAQLLTTIDFDGDTVGLAYVR

SEQ ID NO: 148 - protein, 13 aa, synthetic construct
VCNSNGQCVDVNR

SEQ ID NO: 149 - protein, 15 aa, synthetic construct
VCNSNGQCVDVNRAY

SEQ ID NO: 150 - protein, 21 aa, synthetic construct
YCTCGAPSCIMADTLSHQPSK

SEQ ID NO: 151 - protein, 15 aa, synthetic construct
YVEFVVVLDHGMYTK

SEQ ID NO: 152 - protein, 12 aa, synthetic construct
SECDIAESCTGR

SEQ ID NO: 153 - protein, 29 aa, synthetic construct
CPTLDHQCISFFGSNAAVAPDVCFDFNLK

SEQ ID NO: 154 - protein, 11 aa, synthetic construct
FSDCSRDEHWR

SEQ ID NO: 155 - protein, 8 aa, synthetic construct
GEGNFYCR

SEQ ID NO: 156 - protein, 21 aa, synthetic construct
HDNAQLLTAIDLDGPTIGLAR

SEQ ID NO: 157 - protein, 9 aa, synthetic construct
IFPCAPQDK

SEQ ID NO: 158 - protein, 10 aa, synthetic construct
IFPCAPQDKK

SEQ ID NO: 159 - protein, 33 aa, synthetic construct
LFCVLGPTGNTISCQSTSSQSDLDIGMVDLGTK

SEQ ID NO: 160 - protein, 19 aa, synthetic construct
LRPGTQCEDGECCEQCQFR

SEQ ID NO: 161 - protein, 16 aa, synthetic construct
NGQPCLNNNGYCYNGK

SEQ ID NO: 162 - protein, 22 aa, synthetic construct
RHDNAQLLTAIDLDGPTIGLAR

SEQ ID NO: 163 - protein, 30 aa, synthetic construct
RHDNAQLLTAIDLDGPTIGLARVGSMCDPK

SEQ ID NO: 164 - protein, 11 aa, synthetic construct
RSTGIVQDHSK

SEQ ID NO: 165 - protein, 23 aa, synthetic construct
SECDIAESCTGQSADCPTDNFQR

SEQ ID NO: 166 - protein, 10 aa, synthetic construct
STGIVQDHSK

SEQ ID NO: 167 - protein, 8 aa, synthetic construct
LIFELGYR

SEQ ID NO: 168 - protein, 15 aa, synthetic construct
LKPGTQCEDGVCCDR

SEQ ID NO: 169 - protein, 10 aa, synthetic construct
AYTSSMCNPR

SEQ ID NO: 170 - protein, 29 aa, synthetic construct
DHSPINLLVGVTMAHELGHNLGMNHDGDK

SEQ ID NO: 171 - protein, 15 aa, synthetic construct
GASLCIMRPGLTPGR

SEQ ID NO: 172 - protein, 14 aa, synthetic construct
ITTNPSVEDHCYYR

SEQ ID NO: 173 - protein, 15 aa, synthetic construct
KRVHELVNTINGFYR

SEQ ID NO: 174 - protein, 17 aa, synthetic construct
KSHDHAQLLTAINFEGK

SEQ ID NO: 175 - protein, 10 aa, synthetic construct
NTLNSFGEWR

SEQ ID NO: 176 - protein, 16 aa, synthetic construct
NTLNSFGEWREADLLR

SEQ ID NO: 177 - protein, 14 aa, synthetic construct
RVHELVNTINGFYR

SEQ ID NO: 178 - protein, 16 aa, synthetic construct
SHDHAQLLTAINFEGK

SEQ ID NO: 179 - protein, 20 aa, synthetic construct
SHDHAQLLTAINFEGKIIGR

SEQ ID NO: 180 - protein, 30 aa, synthetic construct
SHDHAQLLTAINFEGKIIGRAYTSSMCNPR

SEQ ID NO: 181 - protein, 27 aa, synthetic construct
SLNIDVSLTDLEIWSDQDFITVQSSAK

SEQ ID NO: 182 - protein, 35 aa, synthetic construct
SVGIVKDHSPINLLVGVTMAHELGHNLGMNHDGDK

SEQ ID NO: 183 - protein, 13 aa, synthetic construct
VFMKYNSDLNIIR

SEQ ID NO: 184 - protein, 13 aa, synthetic construct
VHELVNTINGFYR

SEQ ID NO: 185 - protein, 15 aa, synthetic construct
YIELVVVADHRVFMK

SEQ ID NO: 186 - protein, 9 aa, synthetic construct
YNSDLNIIR

SEQ ID NO: 187 - protein, 10 aa, synthetic construct
YNSDLNIIRK

SEQ ID NO: 188 - protein, 10 aa, synthetic construct
VTLDSFGNWR

SEQ ID NO: 189 - protein, 14 aa, synthetic construct
AMVTKNNGDLDKIK

SEQ ID NO: 190 - protein, 19 aa, synthetic construct
CADGKVCSNGHCVDVATAY

SEQ ID NO: 191 - protein, 21 aa, synthetic construct
DNSPGQNNPCKMFYSNEDEHK

SEQ ID NO: 192 - protein, 17 aa, synthetic construct
ENGNKIPCAPEDVKCGR

SEQ ID NO: 193 - protein, 9 aa, synthetic construct
FSKSGTECR

SEQ ID NO: 194 - protein, 15 aa, synthetic construct
FVELFLVVDKAMVTK

SEQ ID NO: 195 - protein, 20 aa, synthetic construct
LKSGSQCGHGDCCEQCKFSK

SEQ ID NO: 196 - protein, 18 aa, synthetic construct
MFYSNEDEHKGMVLPGTK

SEQ ID NO: 197 - protein, 18 aa, synthetic construct
SGSQCGHGDCCEQCKFSK

SEQ ID NO: 198 - protein, 24 aa, synthetic construct
SGSQCGHGDCCEQCKFSKSGTECR

SEQ ID NO: 199 - protein, 32 aa, synthetic construct
SGTECRASMSECDPAEHCTGQSSECPADVFHK

SEQ ID NO: 200 - protein, 11 aa, synthetic construct
NQKGDDDGYCR

SEQ ID NO: 201 - protein, 12 aa, synthetic construct
NQKGDDDGYCRK

SEQ ID NO: 202 - protein, 17 aa, synthetic construct
KRHDNAQLLTAIDFDGR

SEQ ID NO: 203 - protein, 15 aa, synthetic construct
GKVCNSNGQCVDVNR

SEQ ID NO: 204 - protein, 9 aa, synthetic construct
YKDDLDKIK

SEQ ID NO: 205 - protein, 11 aa, synthetic construct
ASQLNFTPEQR

SEQ ID NO: 206 - protein, 11 aa, synthetic construct
AYTSSMCNPRK

SEQ ID NO: 207 - protein, 36 aa, synthetic construct
KSVGIVKDHSPINLLVGVTMAHELGHNLGMNHDGDK

SEQ ID NO: 208 - protein, 18 aa, synthetic construct
RKSHDHAQLLTAINFEGK

SEQ ID NO: 209 - protein, 30 aa, synthetic construct
SYEFSDDSMGYYQSFLNQYKPQCILNKPLR

SEQ ID NO: 210 - protein, 350 aa, Anolis carolinensis
MKSLIALVLLGQILASKATSEPFPPLVSSLPDCDDPESEAAANFAVDHINSHSLHGYKYTLSRIENVKVLPRRPTGKIYLLELDLAETKCHVLSPTPIQNCSVRAKVEHAVEGDCDVKMLHLDGQYKVLSTKCHSSPDSREDFEKVCPDCAPLALLDDVNVVNAVNTALAQYNTYNSTDHHFELLEIARGRNSHMPPGTFVEFAIAATNCTEQEAKEHKDCHVMTGEHAQFGFCKATIFKKPSGEGPALFAIFPNVVICDIFDKQVGHSHTHLTKHHLGKKIPSPGAGYSVLDLIHSHNNTLASHESHSAEVPVVQASPVVKRAAEVPPVIQPALPHLQQCPGKYRHFDI

SEQ ID NO: 211 - protein, 345 aa, Boa constrictor
MNSLIALVLLGQILGCTLSHHLGLSLDCNSKEAEQYAEQAVHHINAHNLHGYKQILNIIEDLNIVHRRPRGEVIFIEVNLLETKCHVLDPTPIENCTVRTQQEHAVETDCDVKLLSDEGITKVVAAKCHSSADSVEDPMKNCAHCSVLMPLNDPHVIEVVEYVLHKHNEQNPGNVYSVLEISRGQHRHEPESFYVEFAIVETNCSGQDAAEGHNGCHAKAAADAHVGFCRATVFRKHTATEKLTDEKFESDCVIFEPKAGESHTHLIEHHFGKNIPSPGHNHTILDLIHSHNDTNASHESHSSEALPEVAAPVVKREVPETVIPPINQPTLPVKLCPGKVHHFKV

SEQ ID NO: 212 - protein, 344 aa, Python bivittatus
MNSLIALVLLGQILGCTLSHHLLPQSDCNSEEAEHYAEEGVHYINTHNLHGYKQTLNIIKDLHVLPRRPHGKVIFTELNLLETRCHVLDPTPVENCTVRTQEEHAVEADCDVKLLSDEGVTKVVAVKCHSSPDSVEDVKKLGPNYPILLPLNDPRVVEVVEYVLHKHNEQQPDHVFEVLEITRGQHKHEPESYYVEFVFVETNCSEQESHDAHHDCHPKAAADAHVGFCKATVFRAHDATGKLTDEKFESDCVLFDQKVGETHVHLIEHHLGKNIPSPGHGHTVLDLAHSHNDTNASHESHSAEVLPEVAAPVVKRDVSPTLPPDDHLTPPVKLCPGKVHHFKV

SEQ ID NO: 213 - protein, 342 aa, Pantherophis obsoletus
MNSLVALVLLGQIIGCTFSHHLLPQLDCNSEEAERLAKLAVDYINEHTLHGYRQALNIIKDAHVVPRRPSGKIIFLELSLLETVCHVLDPSPLEKCTVRPQKYHAVEGDCDVKILSDEGIEKVVAVKCHSEPDSAEDVLFNCPICPILLPRNDPHVVESVEYVLHKHNEQLTGHSYEVLEISRGQHKLEPEAYYVEFAIVETNCSAKEAHDNHHDCHPKAAGEGHLGFCRATVFRSQAATEKPKDEKYESDCIIFDVKVGIPHAHLIEHHYAKNIVSPGHNNTVLDLVHSHNHTSASHETHSHEHAATVAAPVVKREAPTEGPHDHTHATNLCPGKVHHFKI

SEQ ID NO: 214 - protein, 342 aa, Thamnophis elegans
MNSLVALVLLGQIIGCTLSHHLVPQFDCNGEEAEKLGNLAVSYINEHSLHGYKHVVNQIKDAHVLPRRPHGKVIFLELDLLETVCHVLDPTPIENCSVRAQQYHAVEADCNVKLVSDEGVDKVVAAKCHSEPDSVEDVRQHCPKCPILLSLSDPHVINSANYVLHKHNDKLPHHAYEVLEISRGQQKFDPEGYYVEFAIVETNCTHQEAHDDNYHCDPKALGEAHFGLCRATVFRSHAATGKPTDEQYESDCVIFDVKDGHSHSHLVEHHFRKNIVSPGYNNTVLSLIHSHNHTSASHETHSHEHGTAIPPHVAKREVPTVPSHDHTHPIKLCPGKVHHFKV

SEQ ID NO: 215 - protein, 343 aa, Thamnophis elegans
MNSLVALVLLGQIIGCTLSQHLEGWQFDCHDEEVEKLARVAVSYINEHSKHGYKQVVNQIKDAYALPQRPHGKINHLELNLLETVCHVLDPTPAENCSVRAQNHHAVEADCHVKLVSDEGVDKVVAAKCHSQPDSVEDVRQHCPKCQILLSLSDPNVIDSANYVLHKHNDKLTHHAYEILEISRGQQKFDPEGYYVEFPIVETNCTHQEAHDDTHHCHPKALGEAHFGFCRATVFRSHAATGDLTDEQYESDCVIFNVKDGHSHSHLVEHHFRKSIVSPGYNNTVLDLVHSHNHTSASHETNSDEYGTAIPPHAGKREVPTVPSHGHALIKLFCPGEIHHFKV

SEQ ID NO: 216 - protein, 342 aa, Thamnophis elegans
MNSLVALVLLGQIIGCTLSHHFGPQFDCNGEEAERFASVAVSYINEHSIHGYKHALNIIKDVHVLPRRPHGKVIFLELDLLETVCHVLDPTPIENCSVRAQQYHAVEADCDVKLLSDEGVDKVVAAKCHSEPDSVEDVRQHCPKCPILLSLSDPHVVDSANYVLHKHNDKLPHHAYEVLEISRGQQKFDPEGYYVEFAIVETNCTHQEAHDDNYHCDPKALGEAHFGLCRATVFRSHAATGKPTDEKYESDCVIFDVKDGHSHSHLVEHHFRKSIVSPGYNNTVLDLVHSHNHTSASHETHSHEHGTAIPPHVAKREVPTVPSHDHTHPIKLCPGKVHHFKV

SEQ ID NO: 217 - protein, 344 aa, Naja naja
MNSLVTLVLLGQIIGCTFSHHLLPQLDCNGEEAENLAELAEQYINEHNLHGYKHTLNVIKDVHVLPRRPHGKVIFLNLEFLETVCHVSDPTPIENCTVRPDYYHAVEAECDVRLLSDEDVVKVVASKCHSEADSVENVRQKCPKCPILIPLNDPHVVQSVEYVLRKYNEEHPEHVFEVLEISRGQHKYDPEAFYVEFAIVETNCSAQEAHDDHHDCHPKAAGEARAGFCRATVFRSHADLEKPKDELYESDCVIFDVKVGHSHTHLIEHHFGKNIPSPGHNNTILDLVHSHNHTSASHESNSHEHVAATEVVAPVAKREAPTAVSHDHTHATKLCPGRVHHFKV

SEQ ID NO: 218 - protein, 343 aa, Naja naja
MNSLVTLVLLGQIIGCTFSVSLLRKFDCNGEEAEELAKVAVKFINEHNLHGYKQTLNIIKEIDVRAPRPKPNVEKVVEIYLNLLETKCHVMDPTPVEKCTVRQQHEHAVEADCLVRIHRELGNKVTGAVCYSKPGSVLKVRQNCTKCPILIPLNDPHVVQSVEYVLRKHNEQLPNHAYEVLEISRGQHQYDPEAFYVEFAIVETNCSAQEAHDDHHDCHPKAAGEAHTGFCRATVFRSHAALEKPKDEKYESDCVIFDVKEGHSHSHLIEHHYGKNIASPGHNNTILDLVHSHNDTDTSNESDSLEAVLDPELLVKTEAPAEIAHDPTKAPPVCPGRVHHFEV

SEQ ID NO: 219 - protein, 343 aa, Naja naja
MNFLVTLVLLGQIIGCTFGTTLSQEFDCNGEEAEKLAKLAVKYINDHNLHGYKQTLNVIKEVDFPAPRPREIVEMVAEMTLNVLETKCHVLDPTPVENCTVRQQHEHAVEAECVVRIHRELGDKVSGANCHSKPGSVGNVRRNCTKCPILIPLNDPHVVQSVEYVLRKHNEQLPNHVYKVLEISRGQHQYDPEAFYVEFAIVETNCSAQEAHDDHHVCHPKAAGEGHTGFCRATVFRSHAALEKPKDEKYESDCVIFDVKKGHSHSHLIDHHYGKNIASPGQNNTVLDLVHSHNHKSSSHESLSVEVMAVPLPVVKREAPAEIAHAHAKAAPLCPGKVHHFKL

SEQ ID NO: 220 - protein, 343 aa, Naja naja
MNFLVTLVLLGQIIGCTFGTTLSQEFDCNGEEAEKLAKLAVKYINDHNLHGYKQTLNVIKEVDFPAPRPREIVEMVAEMTLNVLETKCHVMDPTPVENCTVRQQHEHAVEAECVVRIHRELGDKVSGANCHSKPGSVGNVRRNCMKCPILILLNDPHVLQSVEYVLRKHNEQLPNHVYKVLEISRGQHQYDPEAFYVEFAIVETNCSAQEAHDGHHVCHPKAAGEGRAGFCRTTVFRSHAALEKPKDEQYELDCVIFDDKKGHSHSHLIDHHYGKNIASPGHNNTVLDLVHSHNHKSSSHESLSLEAMTVPLPVVKREAPAEIAHAHANAAPLCPGKVHHFKL

SEQ ID NO: 221 - protein, 343 aa, Naja naja
MNFLVTLVLLGQIIGCTFGTTLSQEFDCNGEEAEKLAKLAVKYINDHNLHGYKQTLNVIKEVDFPAPRPREIVEMVAEMTLNVLETKCHVLDPTPVENCTVRQQHEHAVEAECVVRIHRELGDKVSGANCHSKPGSVGNVRRNCTKCPILIPLNDPHVVQSVEYVLRKHNEQLPNHVYKVLEISRGQHQYDPEAFYVEFAIVETNCSAQEAHDDHHVCHPKAAGEAHTGFCRATVFRSHAALEKPKDEKYESDCVIFDVKKGHSHSHLIDHHYGKNIASPGQNNTVLDLVHSHNHKSSSHESLSVEVMAVPLPVVKREAPAEIAHAHAKAAPLCPGKVHHFKL

SEQ ID NO: 222 - protein, 344 aa, Hydrophis curtus
MNSLVTLVLLCQIIGCMFSHHLSPQLDCNGEEAEKLAELAVHYINKHNLHGYKQALNVIKDVHVLPRRPSGKVIFLELDLLETECHVLDPTPVENCTVRPQHYHAVKGDCDVKLLSDEGINKVVASKCHSDPDSVEDVRQNCLNCPILIPLNDPHVVQSVEYVLHKHNEKQPSHAYEVLEISRGQHKYEPEAFYVEFAIVETNCSAQEAHDDHHDCHPKAAGEAHIGFCRATVFRSHATLEKPKDEQYESDCVIFDVKEGHSHTHLIEHHFGRNIASPGENNTVLDLIHSHNHTSASHESHSHEHMVIPVVAAPVAKREAPTEISHDHTHPPKLCPGKVHHFKV

SEQ ID NO: 223 - protein, 343 aa, Hydrophis curtus
MNSLVTLVLLAQIIECMFSVVLLKESDYNGEEAKKYGDLAVHYINQHNLHGYKQTLNVIKRVDFLPPPYVEKVVSVELDVLETECHVLDPTPVENCTVRQQDHHAVKSECVVRIHRENGDKVFGANCHSKPDSVENVRQNCSKSPILVPLNDPHVVQSVEYVLHKHNEKQPSHAEVLEISRGQHQYEPEAFYVEFAIVETNCSVQEAHDDHHDCHPKAAGEAHIGFCRATVFRSHAALEKPKDEQYESDCVILDVKEGHSHTHLIEHHYGKNIPSPGHNSVVLDLAHSHNHTSSSHESHSQERVVEVIVLPLPVAKREAPTETAHDHAKAAPLCPGKVHHFKL

SEQ ID NO: 224 - protein, 345 aa, Hydrophis curtus
MNSLVTLVLLCQIIGCTLSLLLRKEFDCNGEEAKKFGDLAVHYINQHNLHGYKQTLNVIKRVDFLATRSPYVEKVVSVELDVLETECHVLDPTLSRIVPVRQQDHHAVKSECVVRIHRENGDKVFGANCHSKPDSVENVRQNCSKSLILVPLNDPHVVQSVEYVLHKHNEKQPSHAEVLEISRGQHQDEPEAFYVEFAIVETNCSAQEAHDDHHDCHPKAAGEAHIGFCRATVFRSHAALEKPKDEQYESDCVIFDVKEGHSHTHLIEHHYGKNIPSPGHNSVVLDLAHSHNHTSSSHESHSQERVVEVIVLPLPVAKREAPTETAHDHAKAAPLCPGKVHHFKL

SEQ ID NO: 225 - protein, 342 aa, Vipera berus
MNSLIALLLLGQIIGCTFSHHLPSHGDCNGEDAKKWAHLAVHYINEHTLHGYKQDLNIIKDIHVLPRRPRGKIIFLELELLETVCHVLDPTPLENCTVRPQHYHAVEGDCDVKIIHDEDVDKVVAAKCHSSPDSVEDVRRNCPKCPILFPLNDPHVVDAIEYVLNKHNEKLSGHVYEVLEISRGQYTFEPEGFYVEFAIVESNCTAQEAQDDHHHCHPNRAGEEHIGFCRATVVRSHASLEKLKDEQFESDCVIFDVKEGHSYSHLIEHHIGKYSTSPGHNNTVLNLAHSHNHTSASHESHSHEHVVEVPVAVAKREIPTNIPPHHTHPVNLCPGKVHHFKV

SEQ ID NO: 226 - protein, 343 aa, Vipera berus
MNSLVALLLLSQIIGSTLSSQVRGDIDCNDKDAKDWAGKAVRYINEHKLHGYKQDLNVIKNIHILPWDGDLVAIYLKLNFLETECHVLDPTPVENCTVRPQHNHAVKIDCDVKIIYDIETHKDDVFVKCNSTPDSVENVQQNCPKCPILLPQNDPHVVDAVEYVLNKHNEKLSGHVYEVLEISRGQHTFEPEGFYVEFAIVESNCTAQEAQDDHHHCHPNTEGEDHIGFCRATVFRSHASLEKPKDEQFESDCVIFDVKKGHAHCYLIEQHVGKDSTSPGDNTNVLNLAHSHNHTRASHESHSDECVVGCPVAFVKKEVPTDISDHDTPSVKGCPGRVLHFNL

SEQ ID NO: 227 - protein, 343 aa, Vipera berus
MNSLVALLLLGQVIGSTLSSQVRGDIDCNDKDAKHWADIAVRYINEHKLHGYKQDLNVIKNIHILTWDRDLVAGYLKLNLLETECHVLDPTPVEKCTVRPQHNHTVEMDCDVKIIYNVMTLKDEVSVKCNSTTDSVENVQRNCPKCPILLPRNDPHVVDAVEYVLNKHNEKLSGHVYEVLEISRGQHTFEPEGFYVEFAIVQSNCTAQEAQDDHHHCHPNTAGEDHIGFCRATVFRSHASLEKPKDEQFESDCVMFDVKEGHTHSYLIEQHVGKDSTSPGDNTTVLNLAHSHNHTRASHEFHSDECVVECPVAFVKKEVPTDISDHHTPPAKGCPGRVLHFKL

SEQ ID NO: 228 - protein, 344 aa, Deinagkistrodon acutus
MNSLVALLLLGQIIGCTFSHHLTSQVDCNGEDAEKWGDMAVHYINEHNLHGYKQALNVIKEIHVLPRRPHGEIVFIEIEVLETQCHVLDETPVENCTVRPQHYHAVGGDCDVKIIHHEGVDKVVGAKCHSDPDSVEDVRRNCPNCPILLPLSDPRVVDCVEYVLSKHNEKLSGHIYEVLEISRGQHQYEPEAFYVEFVIVEVNCTAQEAHDDHHHCHPNTAGEDHIAFCRATVFRSLASLEKPKDEKFESDCVILNVKEGHAHSHLIQHHVGKYSISPGHNNTVLNLAHSHNHTSASHESHSHEHVAEVPVAVAKREVPTDIPHHHMHSHPVKLCPGKVHHFKL

SEQ ID NO: 229 - protein, 341 aa, Deinagkistrodon acutus
MNSLVALLLLGQIIGSTLSSQVRGDLECDNKDAKEWADVAVRYINEHKLHGYKHALNVIKNIVVVPWDGDLVAVFLKLNLLDTECHVSDSTPVENCTVREQHNHAVEMDCDVKIMFDIETFKRDVFVKCHSTPDSVENVRRDCPKCLILLPPNDPRVVDSVEYVLSKHNEQLSGHIYEVLEILRGQYKYEPEAFYVEFVIVEVNCTAQEAHDDHHHCHPNTAGEDHIAFCRATVFRSHVGLEKPKDKKFESDCVILHVKDGHAHSHLIQQHIEKNSISPEHNITILNFIHPQNHTSTSHESHEHAAEVPVDFVKKELPTDISNRHTPPVKGCPGKVHHFEL

SEQ ID NO: 230 - protein, 288 aa, Deinagkistrodon acutus
MNSLVALLLLGQIIGSTLSYLVKEEDLNCNSKAAKYRADQVVRYINDHKLHGYKQALNVIKIFHLLSPERFSTVFYLKLNLLDTECHVLDQTPVEKCTVRPQHNHAVEMDCDAKIIFNYDIYKDYIFVKCNSTPYEPEAFYVEFVIVEVNCTAQEAHDDHHHCHPNTAGEDHIAFCRATVFRSHASLEKPKDEKFESDCVILNVKRGHAYSQLIQHHVEKNSISPGHNSTVLNLAHSHNHTRASHESHSDECVVECPVAFVKKNIQTNIPHHTHPVEGCPGRVFHFQL

SEQ ID NO: 231 - protein, 307 aa, Deinagkistrodon acutus
MNSLVVLVLLGQVIGSTHSSMVKEEDLNCNSKAAKYRADQAVDYINEHNLHGYKQALNVIKIIRLLPSDGLSVIFNLKLNLLETKCHVLDPTPVENCTVRPQLNHAVETDCDVKIIYNIATFKDEFLVKCHSTPGSVENILRDCPKCPLLLPLSDPHVVDSVEYVLSKHNEKLSGHIYEVLEISRGQHQHIAFCRATVFRSHASLEKPKDEKFESDCVILHVKGGHAHSHLIQHHTGKYSTSPGHNSTILNVAHSHNYTSASHESHSHEHVAEVPVDSVKKEVPTDIPHHHTPPVDGCPGKVLHFKL

SEQ ID NO: 232 - protein, 344 aa, Protobothrops flavoviridis
MNSLVALLLLGQIIGCTFSHHLPSHVPVDCNGEDAEKWADLAVDYINEHNLHGYKYAVNVINEIFVLPRRPHGKMILLELNLLETECHVLDQTPIKNCTVRPPHYHAVEGDCDVKIIHDEDVDKVVAAKCYSNPDSVEDVQQNCPKCPILLSLTDPHVVDSVEYVLEKHNAKLSGHIYEVLEISRGQHKYEPEAYYLEFVIVEVNCTAQEAHDDHHQCHPYTAGEEHIGFCRATVFRSHASLEKPKDEKFESDCVIFDIKEGHAHSHLIEHHVGKYSTSPGYNNTVLNLVHSHNHTSASHESHSHEHVAEVPVAVAKREVPTDIPHDHTHPVKLCPGKVHHFKL

SEQ ID NO: 233 - protein, 324 aa, Protobothrops flavoviridis
MNSLVALVLLGQIIGSTVSFQLGPNMDCNTKGTKDWADIGVHYINEHKLHGYKQALNVIKIFRLLPSDGRSVIFHFNLNLLETECHVLDSTPVENCTVRPQHNHAVEMDCNVRIIHDITTFEDEVFVKCSSTPGSVENILRDCPKCPILLSPNDPHVVDSVEYVLNKHNEKLSGHIYEVLEISRGQHKYEPEAYYLEFVIVEINCTAQEAHDDYHQCHPYTAGEDHIAFCRSTVFRSHASLEKPKDEKFESDCVILDVKEGHSHSHLIEHHVGKYSTSPGYNSTDECVVECPVAFVNKEVPTDISDHNTPPVKGCPGRVLHFQL

SEQ ID NO: 234 - protein, 341 aa, Protobothrops flavoviridis
MNSLVALVLLGQIIGSTHSYMVKEEDLNCNSKGAKYADQAVRYINEHNLHGYKQALNVIKIFRLLPSAGFSVIIYLKLNLLETECHVLDSTPVENCTVRQQHNHAVETDCDVKIIYNIMTFKDEFLVKCHSTPGSVENILRDCPKCPILLSLSDPHVVDSVEYVLEKHNAKLPGHIYEVFEISRGQHKYEPEALYLEFVIVEVNCTAQEAHDDHHQCHPYTAGEDHIAFCRSTVFRSHASLEKPKDEKFESDCVILDVKEGHAHSHLIQQHIEKNSISPEHNITILNFVHPHNHTSTSHESHEHVADVPVAFVKKELPTDISDHHTTPVKGCPGKVHHFEL

SEQ ID NO: 235 - protein, 342 aa, Gloydius blomhoffii
MNSLVALVLLGQMIGSTLSHHLQSHVDCNGEDAEKWADMAVHYINEHNLHGYKQVFNVINEIHVLPRRPRGKIIILELKLLETECHVLDPTPVENCTVRPPHYHAVEGDCDVKILHDEGVDKVIGAKCHSDPDSVEDVRRNCPKCPILLPLSDPHVVDSVEYVLNKHNEKLSGHVYEVLEISRGQHKYEPEAFYVEFAIVEVNCTAQEAHDDHHHCHPNTAGENHIGFCRATVFRSHASLEKPKDEQFESDCVIFDVKEGHAHSHLIEHHIGNYNTSPGHNNTVLNLAHSHNHTSASHESHSHEHVAEVPVAVAKREVPTNTPHDHTHPVKLCPGKVHHFKL

SEQ ID NO: 236 - protein, 324 aa, Gloydius blomhoffii
MNSLVALVLLGQIIGSTLSFQLGPNMDCNTKGTKDWADIGVRYINEHKLDGYKNALNIIKIFRLLPSDGRSVIVHFKLNLLETKCHVLDPTPVENCAVRQQHNHAVEMDCNVRIIHDIATFEDEVFVKCKSTPDSVENVRRNCPKCPILLPPNDPHVVDSVEYVLNKHNEKLSGHVYEVLEISRGQHKYEPEAFYVEFAIVEVNCTAQEARDGHHQCHPYTAGEDHIAFCRATVFRSHASLEKPKDENFESDCVILDVKEGHAHSHLIQQHIEKYSTSPGHNSTDEYVVECPVAFVEKEVPTDMSDHDTPPVKGCPGRVLHFQL

SEQ ID NO: 237 - protein, 342 aa, Bothrops jararaca
MNSLVALVLLGQIIGSTLSHHLQSHVDCNGEDAEKWAHMAVHYINEHNQHGYKCALNVINEIRLLPRRPHGTIVFLKLKVLETECHVLDPTPTENCTVRPQHYHAVGGDCDVKIIHEEGGDKVIGAKCYSDPDSVEDVRRNCPKCPILLNLNDPQVVDSVEYVLNKHNEKVSGHVYEVLEISRGQHKNEPEAYYVEFAIVEVNCTAQEAHDDHHQCHPNTAGENHIGFCRATVFRSHASLEKPKDEQFESDCVIFDVKDGHAHSHLIEHHVGKYSTSPGHNNTVLNLVHSHNHTSASHESHSHEHVTEVPVAVAKREVPKDVPHDHTHPVKLCPGKVHHFEL

SEQ ID NO: 238 - protein, 341 aa, Bothrops jararaca; identical to SEQ ID NO: 4

SEQ ID NO: 239 - protein, 342 aa, Bothrops jararaca
MNSMVALVLLGQIIGSTLSSQVRGDLPCDDEDSKWWADVGVRYINEHKLHGYKYALSVIKNIVVVPWDGDWVAVFLKLNLLETECHVLDPTPVKNCTVRTQHNHAVEMDCDVKIMFNVETFKEDVFAKCHSTPDSVENVRRNCPKCPILLPSNNPQVVDSVEYVLNKHNEQLSDHVYEVLEISRGQHKYEPEAYYVEFAIVEVNCTAQEAHDDHHQCHPNTAGEDHIGFCRATVFRSHASLEKPKDEQFESDCVILHVKKGHAHSHLIQQHVEKDSISPEHNNTALNFVHPHNDTRASHESHEHLAKVPVAFVKKELPKDISDRHTTPVVGCPGLRAVGQPY

SEQ ID NO: 240 - protein, 339 aa, Bothrops jararaca
MNSLVALVLLGQIIGSTHSYLVKEEDLNCNSKAAKYRADQAVHYINEHKLHGYKYALNVIKIFHLLPPEEFSTVFYLKLNLLETECHVFDPTPVENCNVRPQHNHAVEMDCDVKIIFNFQFFKTEVFVKCNSTPDSVENVRRNSPKCSILLPPNDPQVIDSVEYVLNKHNEQLSGHVYKVLEISRGQHKYEPEAYYVEFAIMEINCTAQEAHDDHHQCHPNTAGEDHIGFCRATVFRSHASLEKPKDEQFESDCVILDVKEGHAHSHLIQQHVEKYSTSPGHNNTALNLVHLHNDTSTSHKSHSGKCPVVFPKKKVPTDIPHHTHPVEGCPGKVLHFEL

SEQ ID NO: 241 - protein, 342 aa, Crotalus tigris
MNSLVALVLLGQIIGCTFSHHLQSQVDCNGEDAEKWADMAVHYINEHNLHGYKYTTNVINEIHVLPRRPHGVIVFLELKVLETQCHVLDPTPVENCTVRPQHYHAVGGDCDVKILHDEGVDKVIGAKCHSDPDSVEDVRQNCSKCPILLSLSDPHVVDSVEYVLNKYNEKLSGHVYEVLEISRGQHKYEPEAFYVEFAIVEVNCTAEEAHDDHHHCHPNTDGENHIGFCKATVFRSHASLEKPKDEMFESDCVIFDVKEGHAHSHLIEHHVGKYSTSPGHNSTVLNLVHSHNHTSASHESHSHEHVMEVPVAVAKREVPIDVPHDHTHPVKLCPGKVHHFQL

SEQ ID NO: 242 - protein, 341 aa, Crotalus tigris
MNSLVALVLLGXSIGSTLSSQVRGDLECDNKEAKEWADMAVRYINEHKLHGYKQALNVIKNILVVPWDGDLVAVYLKLNLLETECHMLDPHPVENCTIRPQNNHAVKMDCNAKIMFDVVTFKQDVFVKCHSTPDSVENVRRNCPKCPILLPWNDSHVVDSVEYVLNKHNEKLSGHVYEVLEISRGQHKYEPEAFYVEFAIVEVNCTAEEAHDDHHHCHPNTAGEDHIGFCRATVFRSHASLEKPKDEKFESDCVILHVKEGYAYSHLLYKQIEKYNVPPEFRNTVLNLTHSHNHTSTSHESHEHVAEVPVAFVKKELPTDISDHDTPPVEGCPGKALHFQL

SEQ ID NO: 243 - protein, 324 aa, Crotalus tigris
MNSLVALVLLGQIIGSTLSFQLAPNMDCNTKGTKDWADIGVRYINEHKLHGYKQALNIIKIFRLLPSDGLSVMFHFKLNLLETECHVLDPTPVENCIVRQKNNHAVEMDCNVRIIHDIATFGDEVFVKCNSTPDSVENVRRNCPKCPILLPPNDPHVVDSVEYVLNKHNEQLSGHVYEVLEISRGQHKYEPEAFYVEFAIVEVNCTAEEAHDDHHHCHPNTAGEDHIGFCRATVFRSHASLEKPKDEKFESDCVILHVKKGHAYSHLIEHHVGKYNTSPGRNSTDECVVECPVAFVNKEVPKDISDHDTPPVEGCPGKALHFQL

SEQ ID NO: 244 - protein, 324 aa, Crotalus tigris
MNSLVVLVLLGQIIGSTHSYMVREEHLNCNSKAAKYRADQAVRYINEHKLHGYKQALNIIKTFHLLPPERFTTVFYLKLNLLETECHVLDPTPVENCTVRPQNNHAVEMDCDVKINFNFEFFKTEIFVKCNSTPNSVENVRRNCPECPILLPPNDPHVIDSVEYVLNKHNEKLSGHVYEVLEISRGQHKYEPEAFYVEFAIVEVNCTAEEAHDDHHHCHPNTAGEDHIGFCRATVFRSHASLEKPKDEKFESDCVILHVKEGHATSHLIEHHTGKYSTSPGRNSTDECVVECPVAFVKKEVPTHIPHHTHPVEGCPGKVFHFQL

SEQ ID NO: 245 - protein, 344 aa, Crotalus tigris
MNSLVVLVLLGQIIGSTLSYMVREEDLNCNSKAAKYRADQAVHYINEHNLHGYKQALNIIKTIRLLPSDGLSVIIYLKLNLLETECHVLDPTPVENCTVRQQNNHAVETDCDVKIIYNIETFKDEFLVKCHSTPGSVENILQDCPKCPILMPLSEPHVTDSVEYVLNKHNEQLSGHVYEVLEISRGQHKYEPEAFYVEFAIVEVNCTAQEAHDDHHHCHPNTDGENHIGFCRATVFRSYASLEKPKVEMSESDCVILDVKEGHAHSHLIEHHVGKYSISPGHNNTVLNLAHSHNHTSASHESHSHEHVTEVPVAVAKREVPTDIPHHHTPSVDGCPGKVLHFKL

SEQ ID NO: 246 - protein, 344 aa, Crotalus horridus
MNSLVALVLLGQIIGSTHSYMVREEDLNCNSKAAKYRADQAVHYINEHNLHGYKQALNIIKTIRLLPSDGLSVIMYLKLNLLETECHVLDPTPVENCTVRQQHNHAVETDCDVKIIYNIETFKDEFLVKCHSTPGSVEKILEDCPKCPLLLSLSDPHVVDSVEYVLNKHNEKLSGHVYEVLEISRGQHKYEPEAFYVEFAIVEVNCTAQEAHDDHHHCHPNTAGENHIAFCRATVFRSYASLEKPKDEKFESDCVILHVKEGHTHSHLIEHHIGKYSTSPGHNSTVLNLAHSHNHTSASHESHSHEHVTEVPVAVAKREDPTDIPHHHTPSVDGCPGKVLHFKL

SEQ ID NO: 247 - protein, 343 aa, Crotalus horridus
MNSLVVLVLLCQIIGSTYSYMVREEDLNCNSKAAKYRADQAVRYINEHKLHGYKQALNIIKIFHLLPPERFMTVFYLKLNLLETECHVLDPTPVENCTVRPQNNHAVEMDCDVKINFNFEFFKTEIFVKCNSTPDSVENVQRNCPECPILLPPNDPHVINSVEYVLNKHNEKLSGHVYEVLEISRGQHKYEPEAFYVEFAIVEVNCTAEEAHNDHHHCHPNTAGKDHIGFCRATVFRSHASLEKPKDEMFESDCVIFDVKKGHAHSHLIQQHIGKYNISPGHNNAVLNLAHSHNDTRASQESHSDECVVECPVAFVKKEVPTHIPHHTHPVEGCPGKVFHFQL

SEQ ID NO: 248 - protein, 324 aa, Crotalus horridus
MNSLVALMLLGQIIGSTLSFQLAANMDCNTKGTKDWADIGVRYINEHKLHGYKQDLNIIKIFRLLPSDGRSVIFHFKLNLLETECHVLDPTPVENCTVRQQNNHAVEMDCNVRIIHDIATFEDEVFVKCNSTPDSVENVRRNCPKCPILLPPNDPHVVDSVEYVLNKHNEKLSGHIYEVLEISRGQHKYEPEAFYVEFAIVEVNCTAEEAHDDHHHCHPNTDGENHIGFCRSTVFRSHASLEKPKDEKFESDCVILHVKEGHAHSHLIEHHVGKYSTSPGRNSTDECVVECPVAFVNKEVPKDISDHDTPPVEGCPGKVLHFQL

SEQ ID NO: 249 - protein, 324 aa, Crotalus horridus
MNSLVALVLLGQIIGSTLSSQVRGDLECDDKEAKEWADIAVRYINEHKLHGYKQALNVIKNILVVPWNGDLVAVFLKLNLLETECHVLDPTPVENCTVRPQNNHAVEMDCDAKIMFDVVTFKTDVFVKCHSTPDSVENVRRNCPKCPILLPRNDSHVVDSVEYVLNKHNEKLSGHVYEVLEISRGQHKSEPEAFYVEFAIVEVNCTAQEAHDDHHHCHPNTTGEDHIGFCRATVFRSHASLEKPKDEKFESDCVIFDVKEGHAHSHLIEHHVGKYSTSPGRNSTDECVVECPVAFVNKELPTDISDHHTTPVKGCPGKVLHFQL

SEQ ID NO: 250 - protein, 342 aa, Crotalus horridus
MNSLVALVLLGQIIGCTFSHHLQSQVDCNGEDAEKWADMAVHYINEHNLHGYKYTNNVINEIHVLPRRPRGEIVFLELKVLETQCHVLDPTPVENCTVRPLHYHAVGGDCDVKIIHDEGVDKVIGAKCHSDPDSVEEVRRNCPECPILLSLSDPHVINCVEYVLNKYNEKLSGHVYEVLEISRGQHKYEPEAFYVEFAIVEVNCTAEEAHDDHHHCHPNTDGENHIGFCRATVFRSHASLEKPKDEMFESDCVIFDVKEGHAHSHLIEHHVGKYSTSPGHNNTVLNLIHSHNHTSASHESHSHEHMTEVPVAVAKREVPTDVPHDHIHPVKLCPGKVHHFKL

SEQ ID NO: 251 - protein, 344 aa, Crotalus scutulatus
MNSLVALVLLGQIIGSTHSYMVREEDLNCNSKAAKYRADQAVHYINEHNLHGYKQALNIIKTIRLLPSDGLSVIIYLKLNLLETECHVLDPTPVENCTVRQQNNHAVETDCDVKIIYNIETFKDEFLVKCHSTPGSVEKILQDCPKCPILLSLSDPHVVDSVEYVLNKHNEQLSGHVYEVLEISRGQHKYEPEAFYVEFAIVEVNCTAQEAHDDHHHCHPNTAGEDHIAFCRATVFRSYASLEKPKDEMSESDCVILDVKEGHAHSHLIEHHTGKYSTSPGHNSTVLNLAHSHNHTSASHESHSHEHVTEVPVAVAKREVPTDIPHHHTPSVDGCPGKVLHFKL

SEQ ID NO: 252 - protein, 342 aa, Crotalus scutulatus
MNSLVALVLLGQIIGSTLSYMVREEDLNCNSKAAKYRADQAVRYINEHKLHGYKQALNIIKTFHLLPPERFTTVFYLKLNLLETECHVLDPTPVENCTVRPQYNHAVEMDCDVKINFNFEFFKTEIFVKCNSTPDSVENVRNCPECPILLPPNDPHVIDSVEYVLNKHNEKLSGHIYEVLEISRGQHKYEPEAFYVEFAIVEVNCTAEEAHDDHHHCHPNTAGEDHIGFCRATVFRSHASLEKPKDEKFESDCVILHVKKGHAHSHLIQQHIGKYNISPGHHNTVLNLAHSHNDTRASQESHSDECVVECPVAFVKKEVPTHIPHHTHPVEGCPGKVHHFKL

SEQ ID NO: 253 - protein, 257 aa, Crotalus scutulatus
DGLSVIFHFKLNLLETECHVLDPTPLENCTVRQQNNHAVEMDCNVRIIHDIATFEDEVFVKCNSTPDSVENVRRNCPKCPILLPPNDPHVVDSVEYVLNKHNEKLSGHIYEVLEISRGQHKYEPEAFYVEFAIVEVNCTAEEAHDDHHHCHPNTDGENHIGFCRATVFRSHASLEKPKDEKFESDCVILHVKKGHAYSHLIEHHVGKYNTSPGRNSTDECVVECPVAFVNKEVPKDISDHDTPPVEGCPGKALHFQL

SEQ ID NO: 254 - protein, 333 aa, Crotalus scutulatus
MNSLVALVLLGQIIGSTLSSQVRGDLECDDKEAKEWADMAVRYINEHKLHGYKQALNVIKNILVVPWDGDLVAVYLKLNLLETECHVLDPTPVENCTIRPQNNHAVKMDCNAKIMFDVVTFKQDVFVKCHSTPDSVENVRRNCPECPILLPRNDSHVVDSVEYVLNKHNEKLSGHVYEVLEISRGQHKYEPEAFYVEFAIVEVNCTAQEAHDDHHHCHPNTAGENHIGFCRATVFRSHASLEKPKDEKFESDCVILHVKEGHAHSHLIEHHVGKYNTSPGRNSTDECVVESSLLTKATGRPVAFVNKEVPADISDHHTTPVKGCPGKVLHFQL

SEQ ID NO: 255 - protein, 342 aa, Crotalus scutulatus
MNSLVALVLLGQIIGCTFSHHLQSQVDCNGEDAEKWADMAVHYINEHNLHGYKYTTNVINEIHVLPRRPHGEIIFLELKVLETQCHVLDPTPVENCTVRPLHYHAVGGDCDVKILHDEGVDKVIGAKCHSDPDSVEDVRRNCPNCPILLSLSDPHVVDSVEYVLNKHNEKLSGHVYEVLEISRGQHKYEPEAFYVEFAIVEVNCTAEEAHDDHHHCHPNTAGENHIGFCRATVFRSHASLEKPKDEKFESDCVIFDVKEGHAHSHLIEHHTGKYSTSPGHNNTVLNLVHSHNHTSASHESHSHEHMTEVPVAVAKREVPTDVPHDHTHPVKLCPGKVHHFKL

SEQ ID NO: 256 - protein, 344 aa, Crotalus atrox
MNSLVALVLLGQIIGSTHSYMVREEDLNCNSKAAKYRADQAVRYINEHNLHGYKQALNIIKTIRLLPSDGLLVVIYLKLNLLETECHVLDPTPVENCTVRQQNNHAVETDCDVKVIYNIETFKDEFLVKCHSTPGSVEKILQDCPKCPILLSLSDPHVVDSVEYVLNKHNEQLSGHVYEVLEISRGQHKYEPEAFYVEFAIVEVNCTAQEAHDDHHHCHPNTAGENHIAFCRATVFRSYASLEKPKDEKFESDCVILHVKEGHAHSHLIEHHTGKHSTSPGHNSTVLNLAHSHNHTSASHESHSHEHVMEVPVAVANREDPTDIPHHHTPSVDGCPGKVLHFKL

SEQ ID NO: 257 - protein, 343 aa, Crotalus atrox
MNSLVALVLLGQIIGSTLSYMVKEEDMNCNSKAAKYRADQAVRYINEHKLHGYKQALNIIKTFHLLPPERFTTVFYLKLNLLETECHVLDPTPVENCTVRPQNNHAVEMDCDVKINFNFEFFKTEIFVKCNSTPDSVENVQRNCPKCPILLPPNDPHVINSVEYMLNKHNEKLSGHVYEVLEISRGQHKYEPEAFYVEFAIVEVNCTAEEAHDDHHHCHPNTAGEDHIGFCRATVFRSHASLEKPKDEMFESDCVIFDVKKGHSHSHLIQQHIGKYNISPGHHNTVLNLAHSHNDTRASHESHSDECVVECPVAFVKKEVPTHIPHHTHPVEGCPGKVHHFKL

SEQ ID NO: 258 - protein, 324 aa, Crotalus atrox; identical to SEQ ID NO: 1

SEQ ID NO: 259 - protein, 324 aa, Crotalus atrox
MNSLVALVFLGQIIGSTLSSQVRGDLECDDKEAKEWADMAVCYINEHKLHGYKQALNVIKNILVVPWDGDLVAVYLKLNLLETECHVLDPHPVENCTIRPQNNHAVKMDCNAKIMFDVVTFKQDVFVKCHSTPDSVENIRRNCPKCPILLPRNDSHVVDSVEYVLNKHNEKLSGHIYEVLEISRGQHKYEPEAFYVEFAIVEVNCTAQEAHDDHHHCHPNTAGEDHIGFCRATVFRSHASLEKPKDEKFESDCVILHVKEGHVHSHLIEHHTGKYSTSPGSNSTDECVVQCPVAFVNKELPTDISDHHTTPVKGCPGKVFHFQL

SEQ ID NO: 260 - protein, 343 aa, Crotalus adamanteus
MNSLVALVLLGQIIGSIHSYMVREEDLNCNSKAAKYRADQAVQYINEHNLHGYKQALNIIKTIRLLPSDGLLVVIYLKLNLLETECHVLDPTPVENCTVRPQNNHAVETDCDVKIIYNIETFKDEFLVKCHSTPGSVEKILQDCPKCPILLPPNDPHVVDVEYVLNKHNEKLSGHVYEVLEISRGQHKYEPEAFYVEFAIVEVNCTAQEAHDDHHHCHPNIAGEDHIGFCRATVFRSYASLEKPKDEKFESDCVILDVKEGHAHSHLIQQHIGKYSTSPGHNNTVLNLAHSHNHTSASHESHSHEHVTEVPVAVAKREDPTDIPHHHTPSVDGCPGKVLHFKL

SEQ ID NO: 261 - protein, 343 aa, Crotalus adamanteus
MNSLVVLVLLGQIIGSTHSYMVREEDMNCNSKAAKYRADQAVRYINEHKLHGYKQALNIIKTFHLLPPERFTTVFYLKLNLLETECHVLDPTPVENCTVRPQNNHAVEMDCDVKINFNFEFFKTEIFVKCNSTPDSVENVRRNCPECPILLPPNDPHVIDSVEYVLNKHNEKLSGHIYEVLEISRGQHKYEPEAFYVEFAIVEVNCTAEEAHDDHHHCHPNTDGENHIGFCRATVFRSHASLEKPKDEKFESDCVILHVKEGHAHSHLIQQHIGKYNISPGHNNTVLNLAHSHNDTRASQESHSDECVVECPVVFVKKEVPTHIPHHTHPVEGCPGKVFHFQL

SEQ ID NO: 262 - protein, 324 aa, Crotalus adamanteus
MNSLVALMLLGQIIGSTLSFQLAGNMDCNTKGTKDWADIGVRYINEHKLHGYKQALNIIKIFRLLPSGGLSVIFHFKLNLLETECHVLDPTPVENCTVRPQNNHAVEMDCNVRIIHDIATFEDEVFVKCNSTPDSLENVRRNCPKCPILLPPNDPHVVDSVEYVLNKHNEKLSGHIYEVLEISRGQHKYEPEAFYVEFAIVEVNCTAEEAHDDHHHCHPNTDGENHIGFCRATVFRSHASLEKPKDEKFESDCVILHVKEGHAHSHLIQHHTGKYSTSPGRNSTDECVVECPVAFVNKELPTDISDHDTPPVEGCPGKVLHFQL

SEQ ID NO: 263 - protein, 324 aa, Crotalus adamanteus
MNSLVALVFLGQIIGSTLSSQVRGDLECDDKEAKEWADMAVRYINEHKLHGYKEALNVIKNILVVPWNGDLVAVFLKLNLLETECHVLDPTPVENCTVRPQNNHAVEMDCDAKIMFDVVTFKKDVFVKCLSTPDSVENVRRNCPKCPILLPRNDSHVVDSVEYVLNKHNEKLSGHVYEVLEISRGQHKYEPEAFYVEFAIVEVNCTAQEAHDDHHHCHPNTAGEDHIGFCRATVFRSHASLEKPKDEKFESDCVILHVKEGHAHSHLIEHHTGKYSTSPGRNSTDECVVQCPVAFVNKELPTDISDHHTTPVKGCPGKVFHFQL

SEQ ID NO: 264 - protein, 342 aa, Crotalus adamanteus
MNSLVALVLLGQIIGCTFSHHLQSQIDCNGEDAEKWADMAVHYINEHNLHGYKYTTNVINEIHVLPRRPHGEIIFLELKVLETQCHVLDPTPVENCTVRPLHYHAVGGDCDVKIIHDEGVDKVIGAKCHSDPDSVENVRQNCPECPILLSLSDPHVIDSVEYVLNKYNEKLSGHVYEVLEISRGQHKYEPEAFYVEFAIVEVNCTAEEAHDDHHHCHPNTDGENHIGFCRATVFRSHATLEKPKDEMFELDCVIFDVKEGHAHSHLIEHHVGKYSTSPGHNNTVLNLIHSHNHTSASHESHSHEHMMEVPVAVAKREVPTDVPHDHTHPVKLCPGKVHHFKL

SEQ ID NO: 265 - protein, 18 aa, synthetic construct
MGWSCIILFLVATATGVH

(19) United States
(12) Patent Application Publication
(10) Pub. No.: US 2024/0190923 A1
(43) Pub. Date: Jun. 13, 2024

(54) BROAD SPECTRUM METALLOPROTEINASE INHIBITOR FOR THE TREATMENT OF SNAKEBITE

(71) Applicant: University of Maryland, College Park, College Park, MD (US)

(72) Inventors: Sean B. Carroll, Chevy Chase, MD (US); Fiona P. Ukken, Washington, DC (US)

(21) Appl. No.: 18/532,999

(22) Filed: Dec. 7, 2023

Related U.S. Application Data
(60) Provisional application No. 63/431,147, filed on Dec. 8, 2022.

(51) Int. Cl. C07K 14/00; A61P 39/02; C07K 16/18

(52) U.S. Cl. CPC C07K 14/001; A61P 39/02; C07K 16/18

(57) ABSTRACT

Provided are compositions and methods for treatment of snakebites. The compositions include a metalloproteinase inhibitor. The compositions can further include one or more antibodies or antigen fragments thereof that bind to components of snake venom. The compositions are administered to an individual who has been envonmated by a snake to thereby reduce damage caused by the envonmation.

### CROSS-REFERENCE TO RELATED APPLICATION

This application claims priority to U.S. provisional patent application No. 63/431,147, filed Dec. 8, 2022, the entire disclosure of which is incorporated herein by reference.

### FIELD

This disclosure relates to compositions and methods for treating snakebites.

### SEQUENCE LISTING STATEMENT

The instant application contains a Sequence Listing which is submitted in .xml format and is hereby incorporated by reference in its entirety. Said .xml file is named “070919_00141_ST26.xml”, was created on Nov. 29, 2023, and is 253,171 bytes in size.

### RELATED INFORMATION

Venomous snakes cause an estimated 120,000 deaths and 400,000 disabling injuries worldwide each year, with approximately 7000 cases of snakebite in the United States. Snakebite is also a serious medical issue in domestic pets, with tens of thousands of dogs being bitten in the U.S. every year. Even though snakebite remains an important global public health threat, there has been very little innovation in therapies. The primary pharmaceutical treatment involves the administration of antivenoms that contain antibodies that have been produced in host animals against whole venoms. But current antivenoms have many drawbacks including variation in or lack of potency, impurities that trigger side effects, and manufacturing inconsistency. Moreover, many countries suffer from a shortage and/or poor quality of antivenoms.

Most snake venoms contain an arsenal of novel toxin proteins that facilitate the immobilization, killing, and initial digestion of prey (3). Resistance to specific venoms has evolved in both snake prey and their predators and such resistance appears to be adaptation to predation pressures, reviewed in (4-6). In certain instances, resistance is conveyed by specific serum proteins that inhibit the biological activity of major toxins such as snake venom metalloproteinases (SVMPs) (5, 7). For example, the Mexican ground squirrel (8), California ground squirrel (9, 10), and New Mexican rock squirrel (11) have evolved resistance to particular rattlesnake venoms via SVMP inhibitors (12).

The ability of prey to evolve venom resistance can trigger a co-evolutionary arms race in which snake predators in turn evolve toxins or venom compositions that circumvent resistance, as has been shown, for example, in the case of California ground squirrels and the Northern Pacific rattlesnake (C. oreganus) (11, 13). Indeed, it is widely thought that the diversity of snake venom composition reflects adaptation to the diversity and susceptibility of prey in their diets (14-17).

However, the potential for venom-prey co-evolution raises yet another facet of possible co-evolutionary pressure on venomous snakes that has not previously received much attention. Many venomous snakes are themselves resistant to their own venom (18-21), reviewed in (5, 22), which has been proposed to protect the snakes from exposure to venom toxins during feeding and digestion (23). Auto-resistance in viperids has been shown to involve serum proteins that inhibit venom hemorrhagic and protease activities (24-26). The specific proteins responsible for venom resistance have been identified in a few crotalids such as Protobothrops flavoviridis (27, 28), Gloydius blomhoffi (29), and Bothrops jararaca (30) that each express a Fetuin-related cystatin-type metalloprotease inhibitor (dubbed “serum factor”) that inhibits autologous SVMP activities. Since the late 19^{th} century, the treatment of venomous snakebite has entailed the administration of antivenoms composed of serum, serum fractions, or purified antibody preparations from animals (typically horses or sheep) hyperimmunized with whole snake venoms (47). However, the antibodies in antivenoms consist of populations of polyclonal antibodies that bind to various venom proteins and act by neutralizing and/or clearing venom toxins from circulation. Because of the nature of their means of production, it is not possible to control the potency of antivenoms against specific toxins. Thus, there remains an ongoing and unmet need to identify new proteins with improved anti-venom properties for use in treating snakebites. The present disclosure is pertinent to this need.

### BRIEF SUMMARY

The complexity of snake venom composition reflects adaptation to the diversity of prey and may be driven at times by a co-evolutionary arms race between snakes and venom-resistant prey. However, many snakes are also resistant to their own venom due to serum-borne inhibitors of venom toxins, which raises the question of how snake auto-inhibitors maintain their efficacy as venom proteins evolve. To investigate this potential three-way arms race among venom, prey, and auto-inhibitors, this disclosure describes the evolutionary origin of serum inhibitors of snake venom metalloproteinases (SVMPs) in the Western Diamondback rattlesnake Crotalus atrox which possesses the largest battery of SVMP genes of any crotalid. The disclosure reveals that C. atrox expresses five members of a Fetuin A-related metalloproteinase inhibitor family but that one family member, FETUA-3, is the major SVMP inhibitor that binds to approximately 20 different C. atrox SVMPs and inhibits activities of all three SVMP classes. The disclosure also demonstrates that FETUA-3 effectively inhibits SVMPs of different rattlesnake species, and that FETUA-3 is a more potent inhibitor of venom metalloproteinase activity than a currently widely used antivenom. The disclosure demonstrates that the fetua-3 gene arose deep within crotalid evolution before the origin of New World species but, surprisingly, fetua-3 belongs to a different paralog group than previously identified SVMP inhibitors in Asian and South American crotalids. Conversely, the C. atrox FETUA-2 ortholog of previously characterized crotalid SVMP inhibitors shows limited activity against C. atrox SVMPs. These results reveal that there has been a functional evolutionary shift in the major SVMP inhibitor in the C. atrox lineage as the SVMP family expanded and diversified in the Crotalus lineage, and supports the use of the FETUA-3 protein as a broad-spectrum inhibitor for therapeutic use. Accordingly, the disclosure provides compositions and methods using FETUA-3 as an anti-venom component. The disclosure supports combining FETUA-3 with other anti-venom agents to improve snakebite outcomes.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. C. atrox venom contains metalloproteinase activity that is inhibited by C. atrox serum. (A) Ten micrograms (μg) of whole C. atrox venom preincubated in the presence (+) or absence (−) of one millimolar (mM) EDTA was assayed for proteolysis of a collagen substrate (Azocoll). The presence of EDTA in the reaction abolishes detectable metalloproteinase proteolysis of collagen. (B) Whole venom proteolysis of collagen is inhibited by C. atrox serum. Error bars indicate standard deviation.

FIG. 2. The fetua gene complex in Crotalus contains five loci. (A-C) The relative positions and orientations of fetua genes (blue arrows) in C. adamanteus (A), C. horridus (B), and C. atrox (C). The C. adamanteus genomic region contains five fetua genes. The C. horridus fetua locus shows the five tandemly arranged fetua genes are flanked by the senp5 and fetub genes. The reference-guided assembly of the C. atrox fetua gene complex also shows five fetua genes.

FIG. 3. Alignment of C. atrox FETUA protein sequences with FETUA inhibitors from other Crotalinae species. C. atrox FETUA amino acid sequences were inferred from the nucleotide sequences of corresponding cDNA clones. A 19-residue deletion occurs in the seventh exon of C. atrox FETUA-2 and FETUA-3, in the same region as a 19-residue deletion in the G. blomhoffi MSF protein. Signal peptide regions are highlighted in grey and alternating exons are marked with black lines above the sequence. Conserved amino acid residues are indicated by asterisks (*). G. blo—Gloydius blomhoffi, P. fl a—Protobothrops flavoviridis, B. jar—Bothrops jararaca, C. atx—Crotalus atrox. The sequences in the alignment of FIG. 3, for each species, listed from top to bottom are SEQ ID NO:3, SEQ ID NO:4, SEQ ID NO:5, SEQ ID NO:6, SEQ ID NO:7, and SEQ ID NO:8, respectively.

FIG. 4. Inhibition of C. atrox proteolytic activity by FETUA proteins. (A) 50% inhibition of whole venom collagen cleavage activity is achieved by ˜2.5 μg of FETUA-3 (green line/lines as signified by arrows) while approximately of 20 μg of FETUA-5 protein is required to achieve 50% inhibition of whole venom collagen cleavage activity (red line). Inhibition of whole venom collagen cleavage activity by FETUA-1, FETUA-2 and FETUA-4 is undetectable. (B) MDC-4 (class III SVMP) proteolysis of collagen is completely inhibited by FETUA-3 while other FETUA proteins show no inhibition but enhance MDC-4 digestion of collagen. (C) FETUA-3 inhibits MPO-1 proteolysis of collagen. Percent residual activity refers to the enzyme activity remaining after incubation with inhibitors. In a 1 ml reaction volume, 1 μg of FETUA-3 is 0.027 μM, 4 μg of MDC-4 is 0.085 μM, and 4 μg of MPO-1 0.157 μM. Experiments were carried out at least three times and error bars indicate standard deviation.

FIG. 5. FETUA-3 inhibits all three classes of C. atrox SVMPs activities on fibrinogen. Inhibition of fibrinogenolytic activity of MDC-4, MAD-3, and MPO-1 by the five FETUA proteins was tested using bovine fibrinogen as substrate. Effects of the FETUA inhibitors on SVMP fibrinogenolysis was assessed by observing cleavage of the α and β chains of bovine fibrinogen as decreased band intensity, the positions of the α-, β- and γ-uncleaved forms are denoted in the control lane in panel. (A-E) FETUA samples incubated with MDC-4, MAD-3 or MPO1, and fibrinogen, and analyzed by SDS-PAGE. Lane 1—fibrinogen alone lane 2-fibrinogen incubated with SVMP; and lanes 3-5—fibrinogen incubated with increasing amounts of FETUA (1, 2.5 and 5 μg). FETUA inhibition of SVMP fibrinogenolytic activity is indicated by asterisk (*). (F) A summary of FETUA inhibition of MDC-4, MAD-3 and MPO-1. FETUA-3 inhibits all three classes of SVMP. FETUA-2 and FETUA-5 show moderate inhibition of MDC-4 activity (β-chain cleavage) while FETUA-2, FETUA-4 and FETUA-5 inhibit cleavage of α and β chains by MAD-3. MPO-1 activity on fibrinogen is only neutralized by FETUA-3.

FIG. 6. FETUA3 binds approximately 20 SVMPs from all three classes. Affinity purification and identification of FETUA-3 interacting venom proteins from C. atrox. (A) SDS-PAGE of C. atrox venom purified on a FETUA-3 affinity column. Lane 1—Protein standards, lane 2—whole venom input, lane 3—flowthrough venom fraction from FETUA-3 affinity column, lane 4—eluate. Asterisks (*) denote enriched proteins in eluate fraction that migrate at the expected sizes of class III and class I SVMPs. (B) Mass spectrometry of the FETUA3 column eluate identified 21 SVMPs based on unique peptide spectra (see Supplementary Data 3). All three classes of SVMPs are represented in the eluate fraction.

FIG. 7. The FETUA-3 protein belongs to a different gene clade/paralog group than other crotalid SVMP inhibitors. A protein phylogeny of full-length FETUA proteins was constructed using sequences from available protein databases and hypothetical translations from our gene models and publicly available genomes. Taxa are represented by color-coded backgrounds: Colubrids—yellow; Elapids—green; and Viperids—grey. Paralog genes in viperid species that are part of a distinct, well-supported clades (bootstrap value >80) are marked with similar colored nodes and arcs: FETUA-1—teal; FETUA-2—orange; FETUA-3—red, FETUA-4—pink; and FETUA-5—purple. C. atrox FETUA family proteins characterized in this study are in blue text, and FETUA proteins that are SVMP inhibitors are highlighted in green. Numbers at the nodes indicate the bootstrap values of 1000 replicates. A complete list of genes, corresponding translations, and accession numbers can be found in Supplementary Data 4.

FIG. 8. The synteny of C. atrox and P. flavoviridis fetua-2 and fetua-3 loci confirms functional shifts in the two paralogs. (A) Schematic showing synteny of the fetua gene complexes of P. flavoviridis and C. atrox (not to scale). Syntenic regions are connected by pink (>90% identity) connecting blocks as determined by (B)LastZ. A fetua-4 gene is not detected in P. flavoviridis due to an ambiguous stretch of nucleotide sequence (dotted line). The contiguous regions are indicated by solid lines. (B) A magnified view comparing the fetua-2 and fetua-3 gene loci in P. flavoridis and C. atrox. The exons are indicated by boxes and arrows indicate direction of transcription. Brown bubbles indicate the positions of shared Gypsy elements, while green bubbles indicate the positions of shared LINE elements as determined by the Repeatmasker program.

FIG. 9. Independent expansions of the fetua gene complex occurred in three snake families. The organization of the fetua locus in fish, mammals, birds, and reptiles. Genes (colored arrows) within the same gene family have the same color (fetua—blue, fetub—orange, senp5—grey, hrg—green). The fetua gene complex is flanked by the fetub to the right and senp5 to the left in all animals examined except for mammals and sharks. Three independent expansions of the fetua locus occurred in snakes (indicated by yellow circles) and yielded multiple fetua genes in colubrids, elapids and vipers. Elephant shark (Callorhinchus milii), mouse (Mus musculus), Chicken (Gallus gallus), Box turtle (Terrapene triunguis), Green anole (Anolis carolinensis), Boa (Boa constrictor), Python (Python bivittatus), Garter snake (Thamnophis elegans), Cobra (Naja naja), Common European adder (Vipera berus) Hundred pacer (Deinagkistrodon acutus), Habu (Protobothrops flavoviridis), Western diamondback rattlesnake (Crotalus atrox).

FIG. 10. Assembly of the fetua gene complex in Crotalus species. (A) Two overlapping BAC clones spanned the fetua locus in C. adamanteus to reveal five fetua genes (blue arrows). (B) The fetua locus in C. scutulatus was retrieved on two separate contigs from the C. scutulatus genome and shows at least five fetua genes and two fetub genes (orange arrows).

FIG. 11. Removal of the His-FLAG tag is necessary for optimal recombinant FETUA activity. (A) Coomassie stained SDS-PAGE of various fractions of recombinant C. atrox FETUA-3 purification. Lane 1—protein standard, lane 2—supernatant from recombinant FETUA-3 expressing cell line, lane 3—flow-through from FLAG purification, lanes 4—affinity purified His-FLAG-FETUA-3 protein. (B) Fluorescent Western blot of purified recombinant FETUA-3 blotted with anti-FLAG (green) and anti-FETUA-3 (red) antibodies. Lane 1—protein standard, lane 2—purified His-FLAG-FETUA-3 protein (yellow, detected by both antibodies), and lane 3—enterokinase digested (untagged) FETUA-3 protein. (C) A comparison of the inhibitory activity of His-FLAG-FETUA-3 and FETUA-3 (tag removed) on C. atrox whole venom proteolytic activity on Azocoll substrate. (D) Coomassie stained SDS-PAGE of recombinant, tag-free C. atrox proteins, Lane 1—FETUA-1, Lane 2—FETUA-2, Lane 3—FETUA-3, Lane 4—FETUA-4, Lane 5—FETUA-5.

FIG. 12. Purification of SVMPs from C. atrox venom. (A) Schematic of MDC-4 purification from venom. (B) Schematic for purification of MAD-3 and MPO-1 from venom. (C) Purified SVMPs from venom analyzed by SDS-PAGE. Lane 1—Protein standard, lane 2—MDC-4, lane 3—MAD-3, lane 4—MPO-1. See Supplementary Data 2 for Mass Spectrometry analyses of purified venom proteins.

FIG. 13. C. atrox FETUA-3 shows cross-species inhibition of metalloproteinase activity. (A) Ten micrograms (μg) of whole C. atrox venom and fifteen micrograms (μg) of C. horridus venom were preincubated with FETUA-3 and assayed for proteolysis of Azocoll substrate. (B) Twenty micrograms (μg) of Sistrurus catenatus edwardsii venom was assayed for metalloproteinase activity on Azocoll in the presence of FETUA-3. The activity of each of the venoms was normalized to the activity observed with two micrograms of C. atrox MDC-4. Error bars indicate standard deviation.

FIG. 14. C. atrox FETUA-3 is a more potent inhibitor of metalloproteinase activity than CroFab antivenom. Two micrograms (μg) of C. atrox MDC-4 was preincubated with FETUA-3 or CroFab and assayed for proteolysis of Azocoll substrate. While ˜50% neutralization was achieved with 0.756 μg of FETUA-3, nearly sixteen-fold more CroFab (12.08 μg) by weight was required to neutralize MDC4 activity to similar levels of inhibition. A similar result was observed with whole C. atrox venom, with approximately 18-fold more CroFab than FETUA-3 required (by weight) to neutralize approximately 50% of whole venom SVMP activity. CroFab used was from Lot #BN201894.

FIG. 15. C. atrox FETUA-3 increases metalloproteinase inhibitory activity when used with CroFab. Two micrograms (μg) of C. atrox MDC-4 was preincubated FETUA-3, CroFab, or a mixture of FETUA-3 and CroFab at one-half IC₅₀ and IC₅₀ concentrations determined previously and assayed on the Azocoll substrate. Addition of FETUA-3 to CroFab improved inhibition of MDC4 activity, with only 17% residual activity detected when combined at their respective IC₅₀ concentrations. CroFab used was from Lot #BN201894.

### DETAILED DESCRIPTION

Unless defined otherwise herein, all technical and scientific terms used herein have the same meaning as commonly understood by one skilled in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification includes every narrower numerical range that falls within such a broader numerical range, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value encompasses variations of +/−10%, +/−5%, or +/−1%.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein are encompassed by this disclosure. Amino acids of all protein sequences and all polynucleotide sequences encoding them are also included, including but not limited to sequences included by way of sequence alignments. Sequences from 95.00%-99.99% identical to any sequence (amino acids and nucleotide sequences) of this disclosure are included.

The disclosure includes all polynucleotide and all amino acid sequences that are identified herein by way of a database entry. Such sequences are hereby incorporated herein as they exist in the database on the effective filing date of this application.

As discussed above, this disclosure provides compositions and methods for treating snakebites. The compositions comprise at least one protein that is at least 95% identical to the sequence of SEQ ID NO:1. In embodiments, the protein is at least 96%, 97%, 98%, and/or 99% identical to the protein of SEQ ID NO:1. In one embodiment, a protein of this disclosure comprises or consists of the sequence of SEQ ID NO:1. The protein of SEQ ID NO:1 is referred to herein from time to time as FETUA-3. Its sequence is: MNSLVALVLLGQIIGSTLSFQLAGNMDCNTKGTKDWADIGVRYINEHKLHGYKQALNIIK IFRLLPSDGRSVIFHFKLNLLETECHVLDPTPLENCTVRQQNNHAVEMDCNVRIIHDIATF EDEVFVKCNSTPDSVENVRRNCPKCPILLPPNDPHVIDSVEYVLNKHNEKLSGHIYEVLEI SRGQHKYEPEAFYVEFAIVEVNCTAEEAHDDHHDCHPNTDGENHIGFCRATVFRSHASL EKPKEEMFESDCVILHVKKGHAYSHLIEHHVGKYNTSPGRNSTDECVVQCPVAFVNKEV PKDISDHDTPPVEGCPGKALHFQL (SEQ ID NO:1). In an embodiment, residues 246 and 248 of SEQ ID NO:1 may be D and K, respectively, wherein SEQ ID NO:1 with the D and K constitutes an allelic variant of FETUA-3.

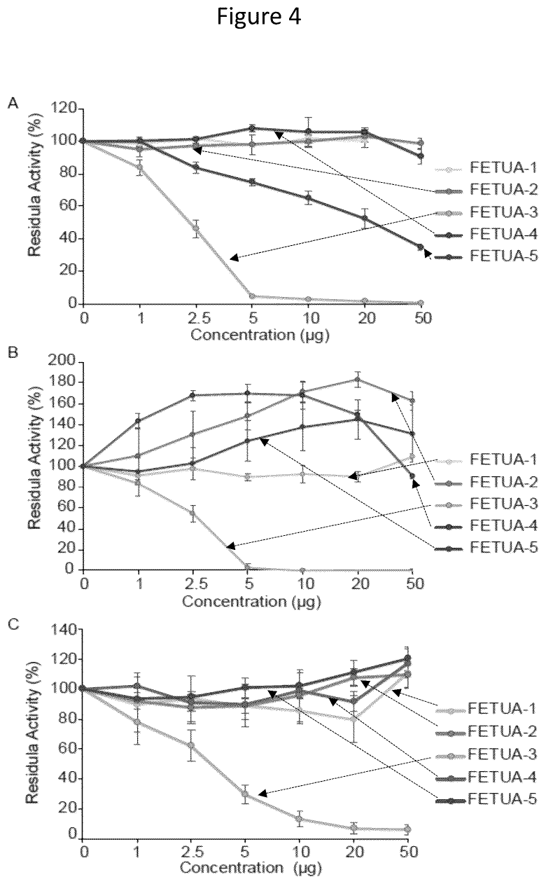

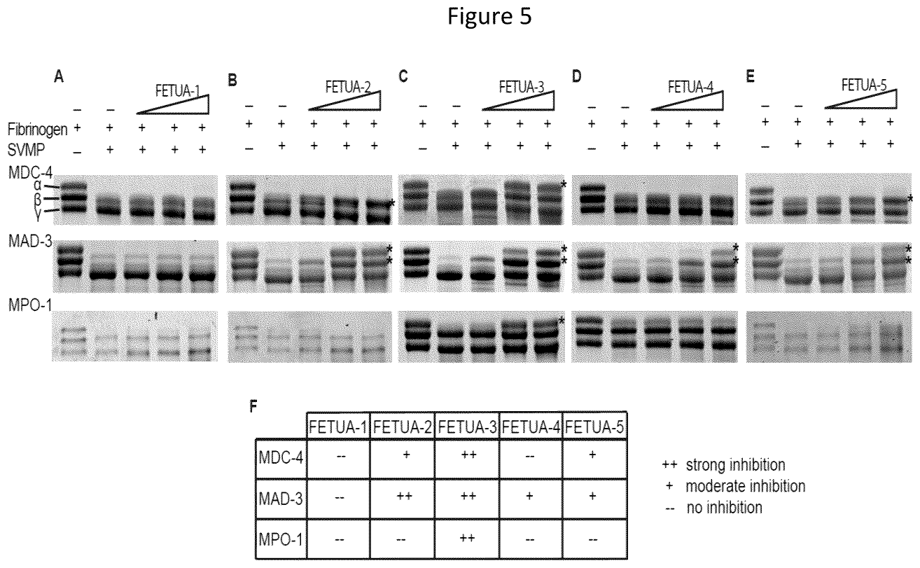

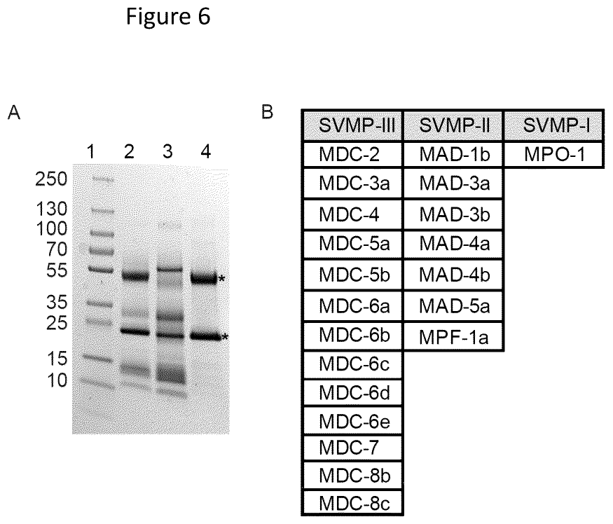

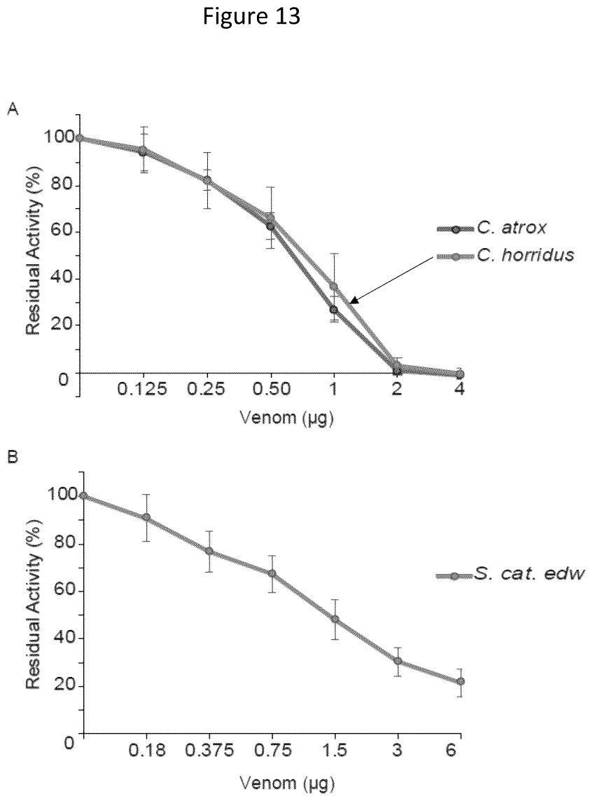

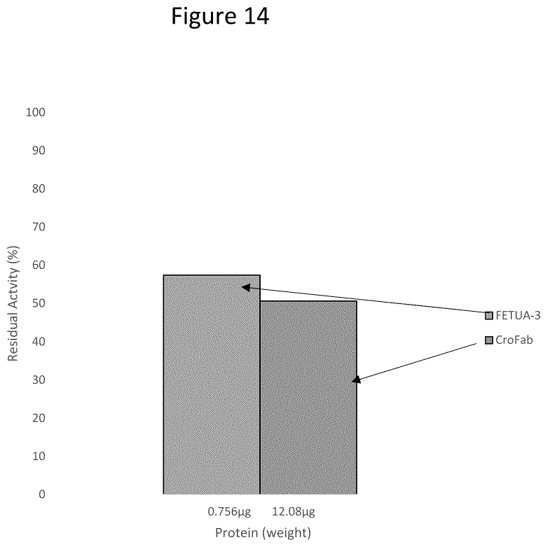

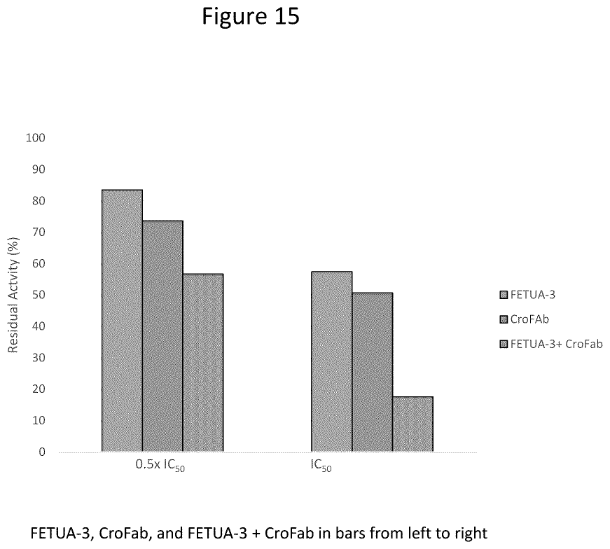

Identification of the protein is further described herein and by way of the Examples. The disclosure includes the unexpected discovery that the FETUA-3 protein is able to neutralize rattlesnake venom metalloproteinases (SVMPs) activity of whole C. atrox venom (illustrated in FIG. 4), is able to neutralize activities of three classes of purified SVMPs (illustrated in FIG. 4 and FIG. 5), and binds to more than 20 C. atrox SVMPs (illustrated in FIG. 6). Further, the disclosure surprisingly reveals the FETUA-3 protein belongs to a different paralog group of the Fetuin protein family than inhibitors that have been previously characterized in Asian and South American snakes. Further still, the present inventors have demonstrated that the FETUA-3 protein is more potent than currently commercially available antivenom at neutralizing the SVMP activities of various species on a weight basis. In a non-limiting example, FIG. 13 demonstrates that C. atrox FETUA-3 shows cross-species inhibition of metalloproteinase activity, and FIG. 14 demonstrates that C. atrox FETUA-3 is a more potent inhibitor of metalloproteinase activity than CroFab antivenom. FIG. 15 demonstrates that C. atrox FETUA-3 increases metalloproteinase inhibitory activity when used with CroFab. Other unexpected aspects of the disclosure are discussed below, and include but are not necessarily limited to the following findings: Among five C. atrox FETUA proteins tested for their ability to inhibit whole venom and purified class I, class II, and class III SVMP enzyme proteolysis of collagen and fibrinogen substrates, only FETUA-3 was a potent inhibitor of all tested SVMP activities. Further, no tested protein besides FETUA-3 significantly inhibited the activity of the major SVMP referred to as MDC-4, and no protein tested except FETUA-3 exhibited inhibitory activity on representatives of all three classes of SVMPs. These and other properties of the FETUA-3 protein as further described herein demonstrate that FETUA-3 is a standalone agent that can by itself reduce snakebite induced injury, such as reduced proteolysis of collagen, fibrinogen, and a combination thereof. Based at least in part on these demonstrations, it is expected that combining the FETUA-3 protein with at least one other anti-venom agent will provide a broadly effective therapeutic approach for treating a variety of snakebites and mitigating a spectrum of deleterious effects the snakebites would otherwise cause. Thus, in embodiments, the disclosure provides compositions and methods that include use of FETUA-3 with one or more other agents to diversify neutralization of an array of snake venom components. In certain embodiments, one or more of the described additional agents can be provided as, for example, small molecule inhibitors, or biologic agents. The small molecule inhibitors include but are not necessarily limited to phospholipase inhibitors, including but not necessarily limited to phospholipase A2 inhibitors, lysophospholipase inhibitors, neurotoxin inhibitors, myotoxin inhibitors, endopeptidase inhibitors, hydrolase inhibitors, factor X activation inhibitors, kininogenase inhibitors, prothrombin activation inhibitors, serine protease inhibitors, thrombin-like enzyme inhibitors, phosphatase inhibitors, heparinase inhibitors, and metal ion chelating compounds. Biologic agents include but are not necessarily limited to peptide inhibitors, antibodies, and fragments of antibodies. Suitable fragments of antibodies include but are not necessarily limited to antigen-binding (Fab) fragments, Fab′ fragments, (Fab′)₂ fragments, Fd (N-terminal part of the heavy chain) fragments, Fv fragments (two variable domains), single domain fragments or single monomeric variable antibody domains, single-chain variable fragment (scFv) and heavy chain only antibodies. Thus, in some examples, one or more monoclonal antibodies or antigen binding fragments thereof that bind to a snake antigen can be combined with the described FETUA-3 protein.

In some examples, an additional agent comprises a polyclonal antibody preparation that comprises a plurality of antibodies or antigen binding fragments thereof, wherein the antibodies or antigen binding fragments thereof specifically bind to a plurality of distinct snake venom antigens. In embodiments, the polyclonal antibodies are obtained from a non-human mammal, such as a horse, goat, or sheep, after the non-human mammal has been immunized with whole snake venom or one or more isolated snake venom components. In embodiments, the described FETUA-3 protein may be combined with crotalidae polyvalent immune fab ovine, sold under the tradename CROFAB. In embodiments, combining a described FETUA-3 protein with another agent increases the therapeutic effect of the combined composition.

A composition of this disclosure is administered to an individual in need thereof. The individual in need is an individual who has experienced a snakebite. In one embodiment, the individual in need is a human. In other embodiments, the individual in need is a canine animal, a feline animal, an equine animal, a bovine animal, or a porcine animal, Thus, the disclosure is suitable for both human and veterinary indications. In embodiments, the individual has been envenomated by any species of rattlesnake, copperhead, or cottonmouth, the latter also referred to as a water moccasin.

In embodiments, an effective amount of a described composition is administered to an individual in need thereof. In embodiments, an effective amount is an amount that reduces one or more signs or symptoms of snakebite, or reduces the severity of injury from the snakebite. An effective amount may also inhibit the development of snakebite-induced injury. A precise dosage can be selected by the individual user of the composition in view of the type of individual to be treated, taking into account such factors as the weight of the individual, the type of the snake, the depth of fang penetration, and the amount of time elapsed between envenomation and administration of the composition. Dosage and administration can be adjusted to provide sufficient levels of the described composition to maintain the desired effect. The compositions may be administered as a single composition, such as a single composition comprising a described FETUA-3 protein, optionally with at least one additional anti-venom agent, or the described FETUA-3 protein and at least one additional anti-venom agent may be administered sequentially. A single dose or a series of doses may be administered. Any suitable route of administration may be used. In general, the described compositions will be administered intravenously, but other routes of administration can also be used.

In embodiments, the described FETUA-3 protein may be provided as a pharmaceutical formulation. The pharmaceutical compositions can be prepared by dissolving, suspending, or emulsifying the FETUA-3 protein in a diluent. Examples of diluents are distilled water for injection, physiological saline, physiologic buffer, vegetable oil, alcohol, and a combination thereof. Further, the compositions may contain stabilizers, solubilizers, suspending agents, emulsifiers, soothing agents, buffers, preservatives, antibacterial agents, and the like. The pharmaceutical compositions may be formulated into a sterile solid or powdered preparation, for example, by freeze-drying, and may be used after sterilization or dissolved in sterile injectable water or other sterile diluent(s) before use. The compositions can include one or more standard pharmaceutically acceptable carriers. Some examples herein of pharmaceutically acceptable carriers can be found in: Remington: The Science and Practice of Pharmacy (2013) 22nd Edition, Pharmaceutical Press.

The described FETUA-3 can comprise conservative or non-conservative amino acid substitutions, insertions, and deletions, and the like, provided the FETUA-3 protein retains a described anti-venom function. In certain approaches, the FETUA-3 protein is modified to exhibit one or more improved therapeutic properties. In embodiments, the protein is stapled, cyclicized, or multimerized. In embodiments, the FETUA-3 protein is provided as a component of a fusion protein. The fusion protein may comprise, in addition to the FETUA-3 amino acids, additional amino acids that improve the efficacy of the protein. In embodiments, the additional amino acids extend the half-life of the fusion protein. In embodiments, the FETUA-3 protein is modified to include additional amino acids used for purification, including any suitable affinity tag, representative examples of which include histidine tags, such as at least four histidines, such as 4, 5, 6, 7, 8, 9, or 10 histidines, biotinylation, glutathione-S-transferase, a FLAG-tag, an epitope tag, and the like. Such modifications may be removed before the protein is used in a described method.

In some examples, the disclosure provides a kit, such as an article of manufacture comprising the FETUA-3 protein and a sealable container in which the FETUA-3 protein, with or without other agents as described herein, is held. In embodiments, the FETUA-3 protein is provided in a solid form such as a powder, film, or gel, for reconstitution and use in a solution. In embodiments, an example kit further comprises printed material that provides instructions for administering the described composition to an individual in need thereof. In some examples, a composition comprising the FETUA-3 protein may be provided with a syringe, optionally with a hypodermic needle. In embodiments, a composition comprising the FETUA-3 protein may be provided in a pre-loaded syringe.

The following Examples are intended to illustrate but not limit the disclosure.

### Example 1

C. atrox Sera Inhibits Venom Metalloproteinase Activity.

Metalloproteinases constitute about half of C. atrox venom protein by weight (34). Auto-resistance to Crotalus venom has been attributed to proteins found in the animal's serum (20), and antihemorrhagic factors that inhibit venom protease activity have been isolated from C. atrox serum (25, 26), but the specific proteins responsible have not been previously identified.

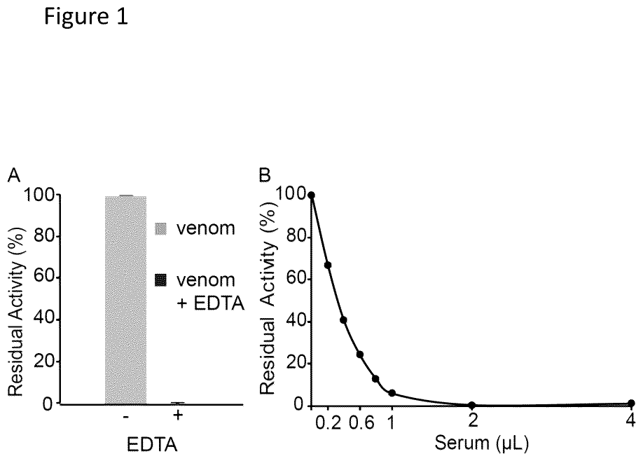

Collagenase activity is widely used as a proxy for the hemorrhagic activities of SVMPs (35). To identify and characterize proteins that confer resistance to metalloproteinase activity in the rattlesnake C. atrox, we used activity on a collagen-based substrate (Azocoll) as an initial assay for metalloproteinases and their inhibitors. Divalent cations such as Zn²⁺ and Ca²⁺ are critical for metalloproteinase function, and chelators such as EDTA inhibit their proteolytic activity (36). To test whether the activity of whole C. atrox venom on Azocoll was sensitive to EDTA, we added one millimolar (mM) EDTA and observed complete inhibition of proteolytic activity, indicating that Azocoll was a suitable substrate for measuring SVMP activity and its potential inhibition (FIG. 1A).

We tested the ability of increasing amounts of whole C. atrox sera to inhibit whole C. atrox venom proteolytic activity and observed dose-dependent inhibition. Just 2 microliters (μl) of sera inhibited the proteolytic activity of 10 micrograms (μg) of whole venom by 97%, thus showing C. atrox sera contains SVMP inhibitory activities (FIG. 1B).

### Example 2

C. atrox Expresses Five Distinct Tandemly Arranged Fetuin-A Genes.

In order to isolate potential serum inhibitors of SVMP activity, we first used a molecular homology strategy. We focused our investigation on potential inhibitors in C. atrox (a North American crotalid).

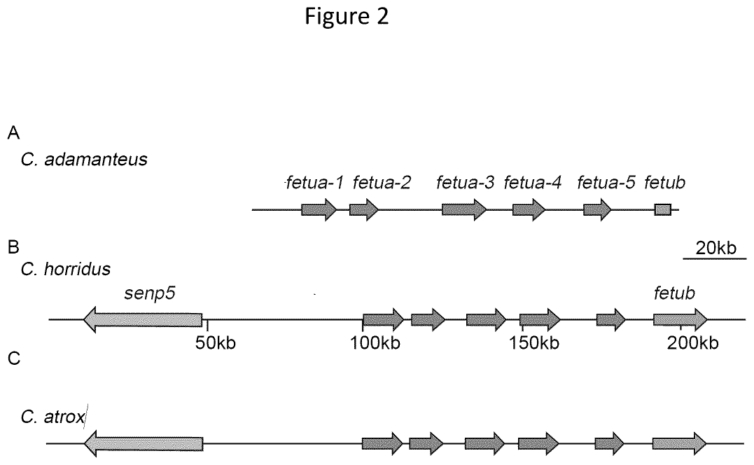

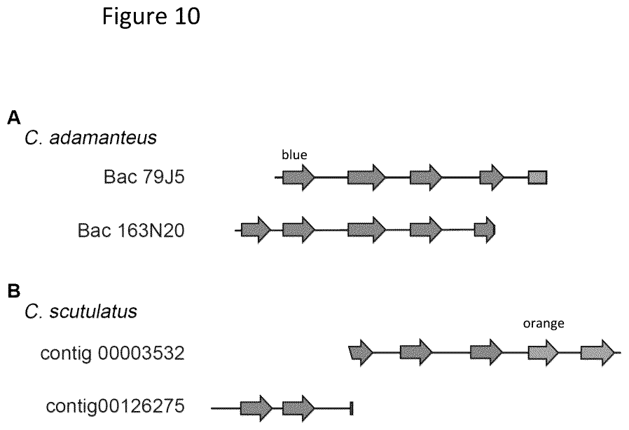

To isolate individual C. atrox fetua genes, we designed RT-PCR primers based on the sequence of Habu Serum Factor (HSF), a well-characterized FETUA family inhibitor from P. flavoviridis. We used C. atrox liver mRNA as a template for RT-PCR and cloned the amplified products into sequencing vectors. Sequencing these cDNA clones revealed three open reading frames (ORFs) encoding fetua genes. To determine whether these ORFs are encoded by separate genetic loci or might be potential isoforms, we screened a C. atrox bacterial artificial chromosome (BAC) library for fetua genes. This library has previously yielded sets of BAC clones that span the large phospholipase A2 and SVMP gene complexes (31, 38). However, we recovered only a single clone that spanned parts of just two fetua genes (apparently due to a gap in the BAC library coverage). Therefore, we endeavored to obtain genomic information from other Crotalus species including C. adamanteus and C. horridus to assess the size of and to annotate the Crotalus fetua region and discovered that it included a five gene complex (FIG. 2A, B; FIG. 10). We then identified the C. atrox fetua gene complex using a reference-guided consensus assembly with the C. horridus fetua complex as the reference (FIG. 2C).

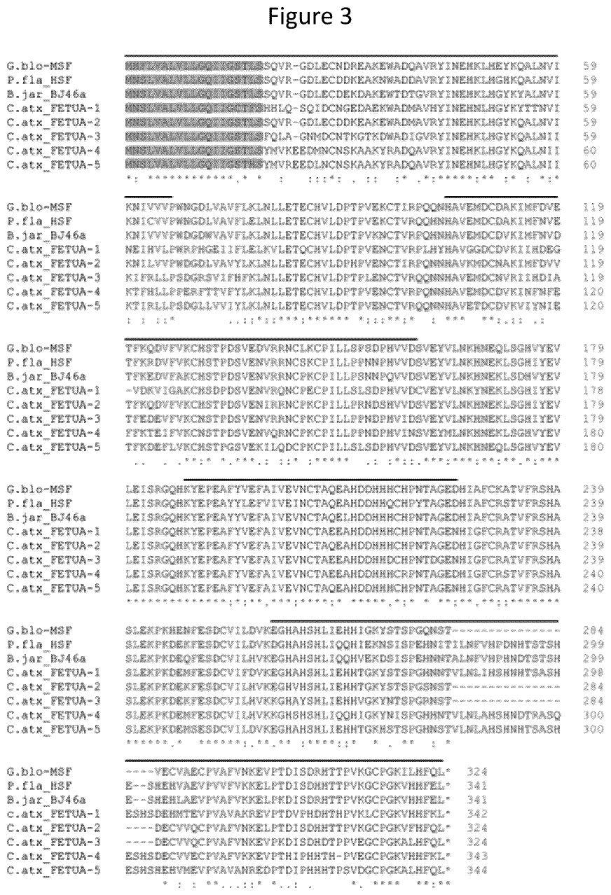

In C. atrox as well as the other three Crotalus species we examined, each of the five tandemly arranged fetua gene loci was comprised of seven exons spanning between 8 and 13 kilobase pairs (kb) and arranged in a head-to-tail fashion. Based on the genomic arrangement, we named the five genes fetua-1, fetua-2, fetua-3, fetua-4, and fetua-5 (FIG. 2). In C. atrox, fetua-1, fetua-4, and fetua-5 encode proteins of approximately 342 amino acids, while fetua-2 and fetua-3 encode proteins that are 19 amino acids shorter due to a deletion in the seventh exon (FIG. 3). A similar 17 amino acid deletion of the 7^{th} exon is observed in the Mamushi Serum Factor (MSF) gene in G. blomhoffi (FIG. 3) (37).

To verify that all five genes are expressed in C. atrox, we used the genomic sequences to design primers to successfully amplify cDNAs corresponding to the two genes that were not recovered in the initial RT-PCR screen of C. atrox liver mRNA. The deduced amino acid sequences of the five encoded proteins exhibit strong similarities to one another and to the three previously identified serum SVMP inhibitors from G. blomhoffi, P. flavoviridis and B. jararaca (FIG. 3).

### Example 3

Only FETUA-3 is a Potent Inhibitor of C. atrox SVMP Activity.

Previous studies of other crotalids have reported no more than three genes in any species such as G. blomhoffi (29). The larger number of fetua genes in C. atrox and other Crotalus species is consistent with the possibility that this apparent expansion could be related to the expansion of SVMP loci (˜30 genes in C. atrox) (31) and an accompanying requirement for a larger number of auto-inhibitors.

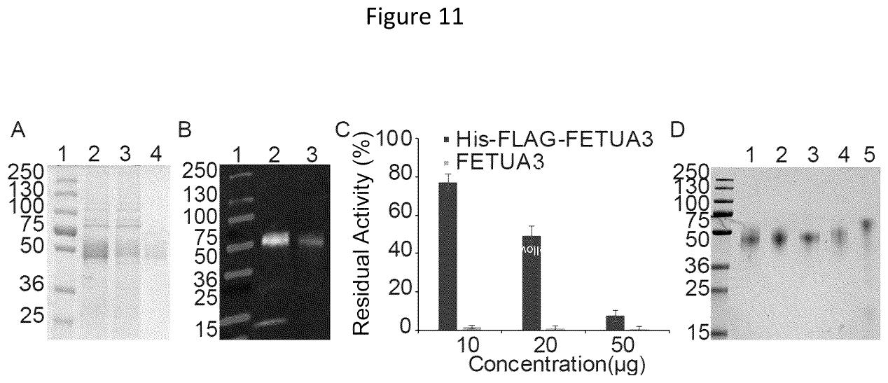

To determine whether each C. atrox FETUA protein was able to inhibit SVMP activity, we expressed recombinant versions of each protein. To facilitate purification, each construct encoded an N-terminal His-FLAG tag. Because the incorporation of a hexahistidine-tag can affect the activity of some recombinant proteins (39, 40), we first examined whether the presence of the His-FLAG tag might affect the activity of FETUA proteins. We performed enterokinase digestion to remove the His-FLAG tag and compared the activity of tagged and untagged/cleaved recombinant proteins in inhibiting C. atrox SVMP activity (FIG. 11A,B). We observed, for example, that tag-free recombinant FETUA-3 protein was significantly more active in inhibiting whole venom SVMP proteolytic activity than the tagged protein (FIG. 11C). Therefore, we used untagged/cleaved recombinant FETUA proteins in all the following analyses (FIG. 11D).

We examined the ability of each of the five recombinant FETUA proteins from C. atrox to inhibit the proteolytic activity of whole venom. The use of collagen for determining proteolytic activity of venom is known as a suitable assay in the art (see, for example, doi:10.1371/journal.pntd.000373). We found that FETUA-1, FETUA-2, and FETUA-4 had no detectable inhibitory activity against whole venom on the Azocoll collagen substrate (FIG. 4A, yellow, blue, and purple lines). In contrast, FETUA-3 showed very strong inhibitory activity with 2.5 μg of protein inhibiting 50% and 5 μg of protein nearly completely inhibiting 10 μg of whole venom collagenase activity (FIG. 4A, green line). FETUA-5 showed much lower SVMP inhibitory activity with ˜50% inhibition observed with 20 μg of protein, respectively (FIG. 4A, red line).

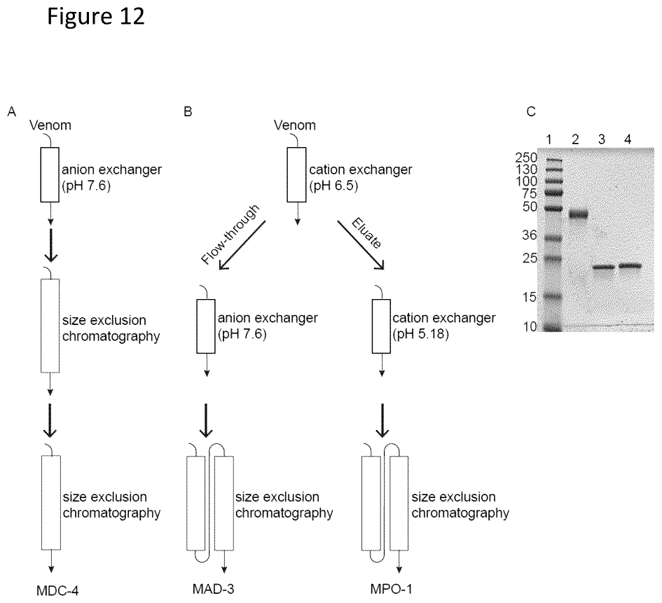

Whole venom is a complex mixture and assays using it will fail to identify FETUA activity against individual components. To further explore potential FETUA activities, we examined their ability to inhibit the activity of purified SVMP enzymes. There are three distinct classes of SVMPs (P-I, P-II, and P-III) that differ by the presence/absence of three distinct domains: all SVMPs contain a metalloproteinase domain; class P-III SVMPs also possess both disintegrin-like and C-terminal cysteine-rich domains; class P-II proteins lack the cysteine-rich domain but possess a disintegrin domain; and class P-I SVMPs possess only the metalloproteinase domain (41, 42). We refer to the P-I class as MPO proteins, the P-II class as MAD proteins, and the P-III class as MDC proteins (31). We purified MDC-4, MAD-3, and MPO-1 proteins, the most abundant class III, II, and class I SVMPs in C. atrox venom, respectively (31) (FIG. 12, Supplementary data. 2). We found FETUA-3 was able to completely inhibit MDC-4 activity (FIG. 4B, green line) and MPO-1 activity (FIG. 4C, green line) on the Azocoll substrate but the other four FETUA proteins exhibited no inhibitory activity on either enzyme in this assay; rather several FETUAs enhanced MDC-4 activity up to approximately 1.8-fold (FIG. 4B). MAD-3 has no activity on Azocoll and therefore inhibition of MAD-3 could not be assessed on this substrate.

However, SVMPs can have different and multiple activities and are known to cleave other substrates such as fibrinogen. We next examined the ability of the five FETUA proteins to inhibit MDC-4, MPO-1, and MAD-3 activity on bovine fibrinogen. We observed that FETUA-3 was a strong inhibitor of all three SVMP enzyme classes on fibrinogen (FIG. 5C, summarized in FIG. 5F). We also detected some activity of other FETUAs: FETUA-2 showed comparable inhibition of MAD-3 as FETUA-3, but very weak inhibition of MDC-4 and no inhibition of MPO-1 (FIG. 5B); FETUA-4 showed modest inhibition of MAD-3 but no inhibition of MDC-4 or MPO-1(FIG. 5D); FETUA-5 showed modest inhibition of MAD-3 and MDC-4 but not MPO-1 (FIG. 5E); FETUA-1 showed no activity against any of the three enzymes tested (FIG. 5A; all FETUA activities are summarized in FIG. 5F).

The potent activity of FETUA-3 and the narrow or absence of activity of the other FETUA family members raised the possibility that FETUA-3 might be the major inhibitor of C. atrox SVMP activity in C. atrox sera.

### Example 4

C. atrox FETUA-3 Binds to at Least 20 SVMPs

Since FETUA-3 is highly effective at inhibiting whole venom proteolytic activity and is able to inhibit representatives of each of the three classes of C. atrox SVMPs, it is possible that FETUA-3 may bind to additional SVMPs in C. atrox venom. To examine this possibility, we employed an affinity chromatography strategy using purified FETUA-3 as a ligand crosslinked to NHS-Trap agarose. We applied whole C. atrox venom to the FETUA-3 affinity column, washed the resin extensively and eluted bound proteins with 0.1M glycine pH 2.7. The venom proteins eluted from the FETUA-3 affinity column were then separated by SDS-PAGE, individual bands were excised from the gel, and both the excised bands and the whole eluate were analyzed by mass spectrometry (FIG. 6A).

The proteins that bound to the FETUA-3 protein affinity resin were identified by unique peptide matches to individual SVMP proteins from a C. atrox venom/SVMP database (29) (Supp. Data 2) (Supp. Data 2). We found that FETUA-3 bound to ˜20 distinct SVMP proteins belonging to all three classes of SVMPs (FIG. 6B). This is nearly all SVMPs that are detectable and distinguishable by mass spectrometry (31). This finding, coupled with the nearly complete inhibition of whole venom activity by FETUA-3, indicates that FETUA-3 is capable of binding to and may inhibit the activity of nearly the entire spectrum of C. atrox SVMPs. We also found that FETUA-3 is a potent inhibitor of SVMP activity from diverse rattlesnake venoms, including C. horridus and Sistrurus catenatus edwardsii (FIG. 13), and a more potent inhibitor of C. atrox SVMP activity than the commercially available CroFab antivenom (FIG. 14). We also found that FETUA-3 increases metalloproteinase inhibitor activity when combined with the CroFab antivenom (FIG. 15).

These results are the first instance in which it has been found that a FETUA protein from any snake species can bind to such a diverse array of SVMPs. This finding is also surprising in light of the expansion of the C. atrox fetua gene complex to five expressed genes, more than are known to exist in other Crotalid genera (37). One might have expected that the larger number of FETUA-type inhibitors would be involved in inhibiting a larger array of SVMPs such as those that exist in C. atrox, but the other FETUAs show limited or no activity against SVMPs. FETUA proteins that lack SVMP inhibitory activities have also been reported in other crotalid species (29). This apparent paradox of possessing more FETUAs but of one protein exhibiting broad activity prompted us to examine the evolutionary relationship of FETUA-3 to previously described SVMP inhibitors.

### Example 5

The FETUA-3 Protein Belongs to a Different Paralog Group than Previously Identified Crotalid SVMP Inhibitors.

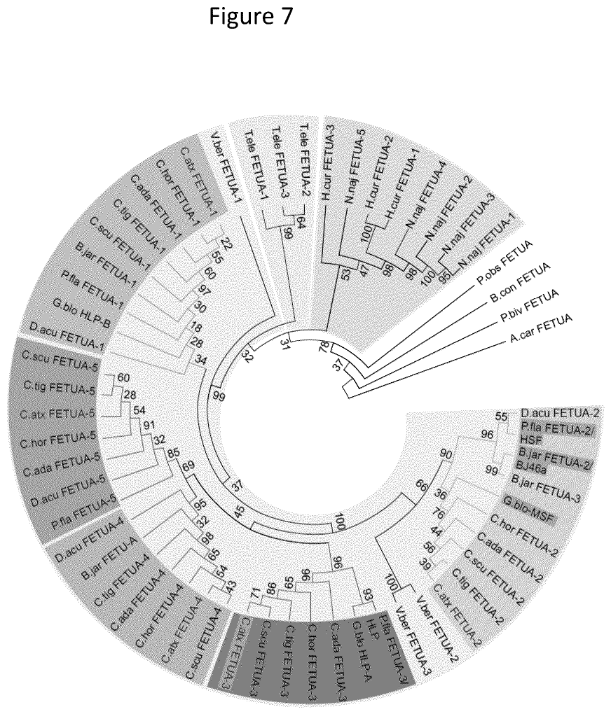

To trace the evolutionary origin of the FETUA-3 and to determine its relationship to other previously identified Crotalid inhibitors—BJ46 (30), HSF (43), and MSF (37) and several Asian crotalid FETUA proteins that lack inhibitory activity—HLP, HLP-A and HLP-B (29), we constructed protein phylogenies using predicted translations of FETUA proteins from published and publicly available genome data from four other Crotalus species, the South American crotalid B. jararaca, and the Asian crotalids D. acutus, P. flavoviridis, and G. blomhoffi (FIG. 7).

Crotalid FETUA proteins formed five distinct monophyletic groups (FIG. 7). To our surprise, however, FETUA-3 nested within a strongly supported clade (BS=96) that excluded all three previously described inhibitors from Asian and New World snakes HSF, MSF, and BJ46a, respectively (FIG. 7; bright red shading). Moreover, the FETUA-3 clade includes two proteins, HLP from P. flavoviridis and HLP-A from G. blomhoffi that have been shown to lack SVMP inhibitory activity (29). Furthermore, the C. atrox FETUA-2 protein—that has relatively limited SVMP inhibition activity, belongs to the clade that includes the inhibitors HSF, MSF, and BJ46a (FIG. 7; light orange shading). Thus, FETUA-3 is paralogous to known SVMP inhibitors, and orthologous to previously characterized non-inhibitors; conversely, the less active C. atrox FETUA-2 is orthologous to all previously identified inhibitors.

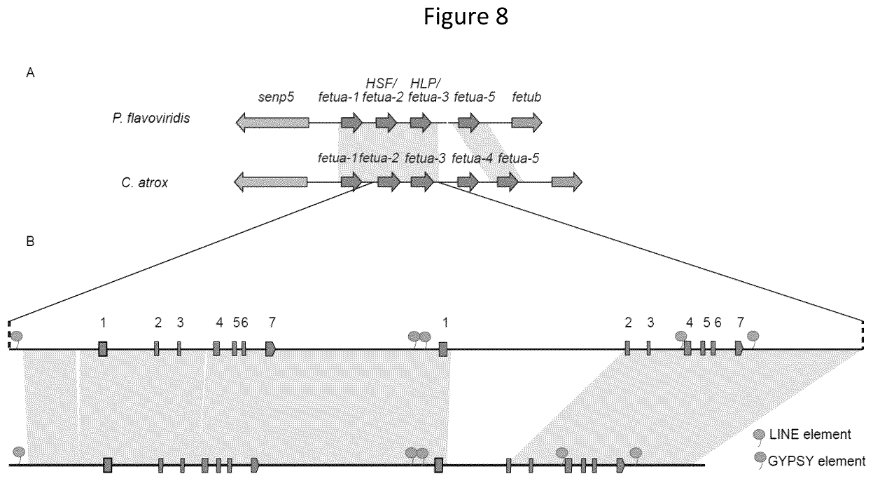

Because these inferences from protein phylogenies are unexpected, we obtained further evidence to confirm the correct identification of key orthologs between species. We assembled and annotated the P. flavoviridis fetua gene region and compared it with the C. atrox fetua gene complex for regions of synteny. Our analysis revealed extensive synteny between the two complexes (except for the absence of fetua-4 gene in P. flavoviridis) and supported an inference of orthology between the P. flavoviridis genes fetua-2 (encoding the SVMP inhibitor HSF) and fetua-3 (encoding the non-inhibitory HLP protein) and the C. atrox fetua-2 and fetua-3 loci, respectively (FIG. 8). Based upon the evolutionary relationships of the Protobothrops, Gloydius, and Bothrops genera, we did not infer that FETUA-2 was the major SVMP inhibitor in their most recent common ancestor. Therefore, and without intending to be bound by any particular theory, we conclude that there has been a functional evolutionary shift among FETUA paralogs during the course of the evolution of the C. atrox lineage from its most recent common ancestor shared with Bothrops, Protobothrops, and Gloydius with the FETUA-3 paralog gaining function as a major inhibitor and the FETUA-2 paralog losing that role.

### Example 6

### Fetua Gene Number Expanded Independently in the Viperid, Elapid, and Colubrid Lineages.

The conserved number of fetua genes in Crotalus irrespective of SVMP gene content, and the only slightly larger number of Fetua genes in Crotalus relative to other crotalids does not support a recent arms race driven by gene expansion and divergence. However, there remains the possibility that the fetua family expanded earlier in crotalid evolution and that the diversity of the gene family has been sufficient to maintain auto-inhibition, for example through functional paralog shifts as shown above for FETUA-3.

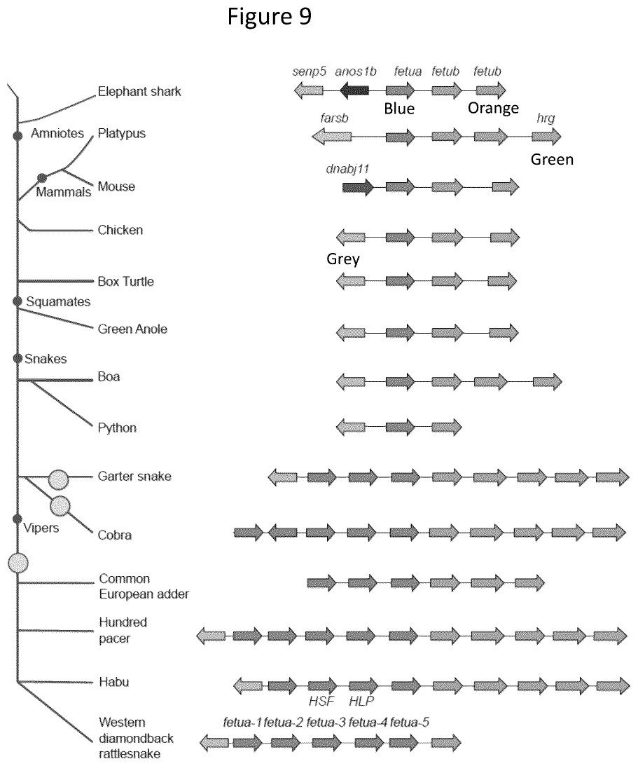

To ascertain the evolutionary history of the fetua gene complex, we annotated genome sequences available from selected non-venomous snakes (python and boa), various venomous snakes (elapid and viperid), and colubrid snakes (which includes non-venomous and venomous species) as well as from other amniotes. In all of the snake genomes analyzed where the flanking sequence was available, the fetua gene complex is flanked by the fetub and senp5 loci as in Crotalus species, which provide useful landmarks for determining the extent of fetua gene expansion. This syntenic genomic arrangement is also conserved in lizards, turtles, and birds (FIG. 9).

We observed that both the python and boa genomes we examined contain just one fetua gene that is flanked by fetub and senp5 genes (FIG. 9). The presence of one fetua gene is also the state in Western Rat Snake (Pantherophis obsoletus), a colubrid (FIG. 7). However, we note that another colubrid, Thamnophis elegans, possesses three fetua genes(FIG. 7, FIG. 9). A still greater number of five fetua genes was observed in an elapid, the Indian cobra (Naja naja) (FIG. 9).

Among viperids, we found at least three fetua genes in Vipera berus (the locus was flanked by fetub gene at the 5′ end of the complex but the genomic sequence 3′ of the complex was not available). In Crotalinae, examination of D. acutus revealed the presence of five fetua genes. In addition, analysis of P. flavovirdis revealed the presence of four fetua genes, including that encoding the inhibitor HSF (43). Furthermore, we were also able to detect at least four genes coding for FETUA proteins in B. jararaca, one of which encodes the inhibitor BJ46a.

The synteny of the fetub and senp-5 loci that flank the fetua (also known as alpha-2-HS-glycoprotein, AHSG) locus in the amniote vertebrates examined suggests that fetua genes were derived from an ancestral AHSG gene and that their expanded number is a snake-specific feature. However, gene phylogenies reveal that this was not a single expansion process. Rather, we find that the five Naja genes belong to a separate clade from the viperid genes and have no known orthologs in other genera (FIG. 7; green shading). Similarly, the three T. elegans genes also belong to a distinct clade (FIG. 7; yellow shading). By contrast, all viperid fetua genes clustered separately from colubrid and elapid fetua family members (FIG. 7; grey shading). Based upon the evolutionary relationships of these species and their fetua genes, we infer that the viperid/crotalid fetua gene complex expanded early in the evolution of the clade and that fetua gene duplications and expansion in elapids and colubrids occurred independently of those in viperids and one another.

### Discussion of Examples

This disclosure relates to the recent discovery of the very large battery of SVMP genes in C. atrox and the hypothesis that the expansion and diversification of the SVMP gene complex selected for co-evolutionary changes in the number or activity of serum SVMP auto-inhibitors. We made three principal findings that bear on this issue which were unexpected. First, we did find a larger number of five fetua genes in C. atrox than the three loci that were known from other crotalid genera, but we determined that there was no recent expansion of the fetua gene family within Crotalus (we also found that other crotalids have at least four fetua genes). Second, of the five expressed C. atrox FETUA-type family members, we showed that only FETUA-3 is a potent, broad-spectrum SVMP inhibitor that binds to most C. atrox SVMPs and inhibits proteolytic activities of proteins from all three SVMP classes. Third, we discovered that there has been a functional evolutionary shift among FETUA paralogs during the divergence of crotalid genera, with the FETUA-3 paralog gaining function as a major inhibitor in Crotalus and the FETUA-2 paralog losing that role. These findings raise several questions concerning how and why a single, but distinct FETUA protein evolved to act as the major SVMP inhibitor in C. atrox, but nevertheless support use of the broad spectrum of FETUA-3 activity in the treatment of snakebite.

### Assessing the Arms Race Hypothesis for the Evolution of Crotalid SVMP Auto-Inhibitors

There are a variety of well-studied examples of evolutionary arms races in which innovation in one species spurs co-evolutionary adaptations in an ecologically interacting counterpart. Many such arms races involve species that produce toxins as defenses against predators or as weapons against prey (44). In such animals, however, it is also the case that the toxin producer must avoid self-intoxication. Thus, auto-resistance is typically a part of the toxic or venomous phenotype and may need to evolve in response to and in step with the evolution of toxins and venoms.

We anticipated that the massive expansion and diversification of the SVMP gene family in C. atrox might have been selected for a co-evolutionary expansion of the fetua-encoded SVMP inhibitors that have previously been identified in other crotalid genera. To evaluate this possibility, it was necessary to identify the full genomic repertoire of fetua genes in C. atrox and to compare it with that of other crotalids (which had not been reported previously and was known only from cDNA sequences) (28, 30, 37). We identified five fetua genes in C. atrox, and the same number of loci in C. adamanteus, C. scutulatus, and C. horridus. Thus, there was no recent expansion of fetua gene number that accompanied the expansion and diversification of SVMPs in the C. atrox lineage.

We also found that the fetua gene complexes in other crotalid genera are larger than was known prior to this disclosure, with four genes in P. flavoviridis, at least four genes in B. jararaca, and five genes in D. acutus. Furthermore, all five D. acutus and C. atrox fetua genes belong to the same five clades, revealing that the five gene fetua complex was established early in a common ancestor and pre-dated the divergence of the genera studied here. Therefore, without intending to be constrained by any particular viewpoint, we infer that fetua gene duplication has not accompanied any arms races that may have unfolded among SVMPs and prey during the divergence of these crotalids. Rather, it appears that four or five FETUA proteins have been sufficient to maintain auto-resistance to SVMPs during crotalid evolution. We therefore analyzed how these proteins maintain auto-resistance in the face of SVMP expansion and diversification.

### One Major SVMP Inhibitor Binds to Most SVMPs and Inhibits all Three SVMP Classes

There is a larger number of structurally and functionally diverse SVMPs than of FETUA proteins in most Crotalus species (31). One potential hypothesis to explain why there are five FETUAs and how they might maintain auto-resistance is by inhibiting different subsets of SVMPs. But that is not what the present disclosure reveals.

We tested the ability of the five C. atrox FETUA proteins to inhibit whole venom and purified class I, class II, and class III SVMP enzyme proteolysis of collagen and fibrinogen substrates. Only FETUA-3 was a potent inhibitor of all SVMP activities—of whole venom, MDC-4, and MPO-1 action on collagen, and of MDC-4, MAD-3, and MPO-1 on fibrinogen. The other four FETUAs largely exhibited weak or no detectable activity in most assays. Only FETUA-2 showed comparable activity to FETUA-3 in any assay, which was limited to the inhibition of MAD-3 action on fibrinogen. Notably, no protein besides FETUA-3 significantly inhibited the activity of the major SVMP MDC-4 (FETUA-2 exhibited only weak, partial inhibition of MDC-4 action on fibrinogen). And no protein except FETUA-3 exhibited inhibitory activity on representatives of all three classes of SVMPs. The broad action of FETUA-3 was further demonstrated by its ability when immobilized on an affinity column to bind to approximately twenty distinct C. atrox SVMPs spanning all three classes.

Without intending to be constrained by any particular theory, what appears to be unprecedented among FETUA proteins examined thus far is C. atrox FETUA-3 broad specificity and strong inhibitory activity against all three classes of SVMPs. In comparison, the well-characterized FETUA-2 proteins from P. flavoviridis and G. blomhoffi fail to inhibit certain class II SVMPs (29). The ability of C. atrox FETUA-3 to interact with and inhibit all three classes of SVMPs indicates that FETUA-3 has evolved certain SVMP protein contacts that other FETUAs lack. Moreover, since other crotalid FETUA-3 proteins generally lack SVMP inhibitory activities, it is considered that FETUA-3 gained some key SVMP contacts in the Crotalus lineage not present in other FETUA-3 orthologs.

### A Functional Evolutionary Shift Between the FETUA-2 and FETUA-3 Paralogs

In addition to the gains of function by the FETUA-3 protein, the very limited SVMP inhibitory activity of C. atrox FETUA-2 relative to its more broadly acting P. flavoviridis, G. blomhoffi, and B. jararaca orthologs reveals that FETUA-2 has lost its role as a major SVMP inhibitor in the evolution of the Crotalus lineage. This evolutionary shift in functions between the FETUA-2 and FETUA-3 paralogs was entirely unexpected.

### Use of FETUA-3 in the Treatment of Snakebite

The ability of FETUA-3 to bind to and inhibit a wide range of SVMPs supports the described incorporation of it as a component of antivenom formulations that are expected to enhance antivenom potency against SVMPs and their myriad clinical effects.

The following Materials and Methods were used to obtain the results in the foregoing Examples.

### Venom, Serum Collection and Processing

Lyophilized C. atrox venom (pool of ˜200 snakes) was purchased from Kentucky Reptile Zoo. Venom stock solution was made by dissolving venom at 10 mg/mL in 20 mM Tris pH 8.0, 1 mM CaCl₂). The solution was filtered through a 0.2 μm Durapore syringe filter and stored at −80° C. until use. C. atrox serum was purchased from the NNTRC, Texas A&M University, Kingsville.

RT-PCR and Cloning of Fetua cDNAs

Total RNA extracted from C. atrox liver samples with the Qiagen RNEasy kit were used to generate cDNA (SuperScript IV First-Strand Synthesis System) according to the manufacturer's protocol. Specific cDNA sequences were amplified using Q5 polymerase (New England Biolabs) and the published Habu Serum Factor (HSF) primer sequence(37). This resulted initially in the isolation of a C. atrox FETUA-3 cDNA sequence encoding the P. flavoviridis C-terminal amino acid sequence KVHHFK instead of KALHFQ at amino acids 317-322. We later isolated full length cDNAs encoding FETUA-1, FETUA-3 and FETUA-5 using oligo-dT primers. Due to the high degree of similarity between the fetua genes, additional primers were required for FETUA-2 and FETUA-4 sequences, and both gene sequences were recovered as two overlapping clones. For a list of primers used refer to Supplementary Data 1A.

Reference-Guided Assembly of the C. atrox Fetua Genomic Region

Pools of clones from a previously constructed whole genome BAC library (5-7× coverage, average insert size of 80-150 kb) for C. adamanteus were PCR screened with fetua-3 primers (Supplementary data 1. B) to identify clones positive for the fetua-3 genomic sequence (38). The University of Michigan DNA sequencing core prepared the Pacific Biosciences sequencing libraries using ten micrograms of BAC DNA according to the standard protocol with a size selection of large (>10000 bp) DNA fragments. The sequencing library for each single BAC clone was sequenced in a single SMRT cell. The raw reads were assembled using the accuracy optimized HGAP2 (Hierarchical Genome Assembly Protocol) algorithm (48).

The C. horridus draft genome assembly has been described previously (49). The fetua-3 gene region was identified by LAST alignment of C. atrox fetua transcripts to the whole genome (50). Manual annotation of the fetua genes on the contig were combined with the crude gene coordinates from transcript alignments and with the inspection of hit coordinates for single exon BLASTs to produce refined gene models (51)

The C. atrox fetua gene complex was identified by alignment (bwa men) of processed (adapter trimmed, overlap merged) whole genome shotgun sequencing reads (˜60× genome coverage) to the C. horridus fetua genomic region (52). Aligned reads were filtered using quality score (samtools -q 20), concatenated (samtools cat), sorted (samtools sort) and used to call nucleotide variants (samtools mpileup -0u -f horridus_fetA.fasta atrox_aligned_sort.bam|bcftools call -mv -0z -o atrox_fetA_region) (53).

The variants were normalized (bcftools norm), filtered by removing adjacent variants within 5 basepairs of each other (bcftools filter -IndelGap 5) and indexed (bcftools index). The indexed variant file was then used to call a reference-based C. atrox fetua consensus genomic sequence (bcftools consensus) (54).

C. atrox Liver Transcriptome and Translation

The C. atrox liver RNA isolation, library preparation, sequencing, read processing and transcript assembly was performed as described previously for venom gland transcriptomes (38). The 201589 assembled liver transcripts were translated using the Trinity helper script (Transdecoder.longestOrf) to obtain 113,718 liver expressed peptides (55).

### Recombinant FETUA Protein Expression and Purification

### Generation of Anti-FETUA-3 Antibodies

A DNA fragment encoding amino acids 78-268 of FETUA-3 was PCR amplified and cloned into the GST-tag expression vector pGEX-6P1. To express GST-cASF-B1 Rosetta-Gami™ 2(DE3)pLacI (Agilent Technologies) cells were transformed with the recombinant construct. GST-FETUA-3 expression was achieved by induction with 1 mM IPTG. Since most of the protein was insoluble, we resorted to isolation of inclusion bodies (56). The recombinant protein containing inclusion body suspension was used to immunize two New Zealand white rabbits (UPFRL). Sera were tested for immunoreactivity on Western Blots and antibodies were affinity purified on His-FLAG-FETUA-3 crosslinked to Aminolink Plus Coupling Resin (Thermo Scientific catalog #20501).

### Gene Syntheses and Plasmid Construction

The sequences coding for C. atrox FETUA proteins were based on sequences from RT-PCR. The endogenous signal peptide sequence (1-19 amino acids) was replaced with a sequence optimized for protein expression (MGWSCIILFLVATATGVHS (SEQ ID NO: 265) in mammalian cell lines. Additionally, a hexahistidine (6×His) encoding sequence tag and a FLAG tag were inserted between the signal peptide and the remaining protein sequence. The synthetized nucleotides were cloned into the mammalian expression vector pcDNA3.4 (Invitrogen, Carlsbad, CA, USA). All constructs were prepared commercially (GenScript, Piscataway, NJ, USA).

### Transfection, Protein Production, and Purification

The recombinant FETUA protein expression plasmids were transiently transfected into either HD 293F/Expi 293F cells or HD-CHO-S cells and FLAG-tag purified recombinant proteins were generated commercially (GenScript). The purified proteins were dialyzed against EK buffer (20 mM Tris, pH 8.0, 50 mM NaCl, 2 mM CaCl₂)) and digested with Enterokinase (EK) light chain (New England Biolabs, P8070L) at a concentration of 0.2 U/μg of recombinant protein. The reaction mix was incubated at 25° C. for 16 hours, following which EK was removed from the reaction mix using an EK Removal kit (Sigma PRKE). The concentrated tag-free recombinant proteins were dialyzed with either Tris-buffered saline (TBS) or phosphate-buffered saline (PBS) and stored at −80° C.

### SDS-PAGE and Western Blotting

Protein samples were resolved by SDS-PAGE using 12% or 15% gels for 30 minutes at 200 V (Mini-Protean Cell; Bio-Rad), electrotransferred (18V for 30 min, Trans-Blot® SD Semi-Dry Transfer or 7 min on Trans-Blot Turbo Transfer System, Bio-Rad) onto Immobilon-FL PVDF membranes (EMD Millipore), blocked with Odyssey® Blocking Buffer (TBS) (Li-Cor) and then incubated with the appropriate primary antibody (mouse anti-FLAG® M2 (1:3000, Sigma) or rabbit anti-FETUA-3 (1:10000)). Membranes were washed with TBS-Tween-20 followed by incubation with the appropriate Alexa Fluor™ 680/790-conjugated secondary antibody (1:15000) in TBS-Tween-20. After further washing with TBS-Tween-20, the membranes were scanned using Odyssey® CLx Imaging system. (Li-Cor).

### Affinity Purification of SVMPs

We used recombinant FETUA-3 protein generated from C. atrox cDNA with the Habu primer that introduced Habu C-terminal amino acid substitutions (P. flavoviridis: KVHHFK: C. atrox: KALHFQ 317-322 aa) for the generation of an affinity column. The C. atrox FETUA-3 with Habu residues 317-322 is indistinguishable from the native C. atrox FETUA-3 protein with respect to inhibition of purified SVMPs or whole venom digestion of Azocoll and fibrinogen substrates. Four milligrams of FETUA-3 was buffer exchanged into coupling buffer (0.2 M NaHCO₃, 0.5 M NaCl, pH 8.3) and covalently bound to a 1 ml NHS-Trap HP column (GE) using ÅKTA prime and the manufacturer's protocol. Two milligrams of lyophilized crude venom was resuspended in 20 mM Tris-HCl pH 7.4, 20 mM CaCl₂), syringe filtered to remove particulate matter and incubated on the FETUA-3-NHS-trap HP column for 1 hour at 37° C. After incubation the column was connected to ÅKTA prime plus and washed with X column volumes 20 mM Tris-HCl pH 7.4, 20 mM CaCl₂) followed by X column volumes 20 mM Tris-HCl pH 7.4, 20 mM CaCl₂), 500 mM NaCl, 0.1% Tween-20 before eluting with 0.1 M glycine pH 2.7. The eluate fractions were collected in a tube containing 1/10 eluate volume of 1 M Tris pH 9. The pH neutralized fractions were pooled and dialyzed against 20 mM Tris, pH 8, 1 mM CaCl₂), concentrated and stored at −80° C. and/or analyzed on 8-16% gradient SDS-PAGE gels with Coomassie staining.

Purification of Metalloproteinases from Venom

### MDC-4 Purification

The purification of MDC4 from C. atrox venom followed the protocol of Williams et al. (57) with the following modifications, 400 mg of C. atrox venom was resuspended in 5 mL of 20 mM Tris-HCl buffer (pH 7.6), filtered through a 0.2 micron (μm) Durapore syringe filter and applied to a 5 mL HiTrap Q XL Sepharose anion exchange column. Column was washed with 20 mM Tris-HCl buffer (pH 7.6) until UV returned to baseline. Protein elution was performed at a rate of 5 ml/min using 20 mM Tris-HCl (pH 7.6), 1 M NaCl gradient (up to 60%) by an ÅKTA FPLC system (GE Healthcare, UK) over 120 ml as 2.5 ml fractions. The collected fractions were run on SDS-PAGE gels, analyzed by Coomassie staining and fractions with the protein of interest (strong band migrating at ˜50 kDa) were pooled. The pooled fractions were concentrated using a 10,000 MWCO Amicon Ultra-15 centrifugal filter and applied to a size exclusion chromatography column (HiLoad 16/600 Superdex 75 μg). Protein elution was performed at a rate of 1 ml/min using 20 mM Tris-HCl (pH 7.6) with 1 ml fractions collected. The fractions were analyzed by SDS-PAGE and Coomassie staining and those containing strong band at ˜50 kDa (MDC-4 and other expressed MDCs) were pooled and concentrated before a second run on the same size exclusion column. Fractions containing the pure protein were pooled, concentrated, and stored as aliquots at ˜80° C. until further use (FIG. 12 C). Protein estimation was performed using Quick Start Bradford 1× Dye Reagent (Bio-Rad) using bovine serum albumin (Thermo Scientific catalog #23208,) as a standard.

### MAD-3 and MPO-1 Purification

C. atrox MAD-3 and MPO-1 were purified in a single protocol that used different buffer pH to separate MPO-1 (˜24.7 kDa) from proteolytically processed MAD-3 (˜19 kDa without disintegrin domain) (FIG. 12 B). C. atrox venom (400 mg) was resuspended in 5 ml of 20 mM Bis-Tris buffer (pH 6.5), filtered through a 0.2 μm Durapore syringe filter and loaded onto a 5 ml HiTrap SP Sepharose cation exchange column. Protein elution was performed at a rate of 5 ml/min using 20 mM Bis-Tris buffer (pH 6.5), 1 M NaCl gradient (up to 60%) by an ÅKTA purifier system (GE Healthcare, UK) over 120 mL as 2.5 ml fractions. The collected fractions were run on SDS-PAGE gels, analyzed by Coomassie staining and fractions with the protein of interest were pooled. The flow-through fraction contained MAD-3 while MPO-1 was present in gradient elution fractions. The flow-through fraction was concentrated using 10,000 MWCO Amicon Ultra-15 centrifugal filter and dialyzed against 20 mM Tris-HCl (pH 7.6) while the MPO-1 containing fractions were dialyzed against 20 mM Piperazine (pH 5.18).

For MAD-3 purification the pooled, concentrated flow-through fractions were loaded onto a 5 ml HiTrap Q XL Sepharose anion exchange column and eluted over a salt gradient (up to 60%) as in MDC-4 purification. Fractions containing MAD-3 were pooled, concentrated in a 10,000 MWCO centrifugal filter and dialyzed against 20 mM Tris-HCl (pH 7.6), 300 mM NaCl before loading onto two size exclusion chromatography columns connected in tandem (HiLoad 16/600 Superdex 75 μg). Elution was performed at 0.5 ml/min and collected as 2.5 ml fractions and those containing protein of interest were pooled and concentrated (FIG. 12C).

The MPO-1 containing fraction (in 20 mM Piperazine (pH 5.1)) was concentrated and the sample (5 mL) was applied to a 5 mL HiTrap SP Sepharose cation exchange column equilibrated in the same buffer. The column was washed with 20 mM Piperazine (pH 5.1) until no protein was detected by the UV detector and eluted by a linear concentration gradient (0 to 60%) of 20 mM Piperazine (pH 5.1), 1 M NaCl under a flow rate of 0.5 ml/min and temperature of 4° C. The eluted MPO-1 fractions were dialyzed against 1×PBS concentrated to a 1 ml sample volume and loaded onto two HiLoad 16/600 Superdex 75 μg columns connected in tandem. Protein elution was performed at a rate of 1 ml/min using PBS (pH 7.2) and 2.5 ml fractions were collected. The fractions were analyzed by SDS-PAGE and Coomassie staining and those containing MPO-1 were pooled and concentrated (10,000 MWCO) and stored as aliquots at −80° C. until further use. Protein estimation was performed using Quick Start Bradford 1× Dye Reagent (Bio-Rad) and bovine serum albumin (Thermo Scientific, catalog #23208) as standards.

### Proteolytic Assays

### Azocoll Assay

Azocoll substrate was suspended at 5 mg/ml in 20 mM Tris-HCl pH 8.0. Working dilutions of venom were prepared from a venom stock solution in 20 mM Tris-HCl pH 8.0 at one mg/ml. Ten micrograms of venom were incubated with increasing amounts of inhibitor candidates in 50 μl microliters and incubated at 37° C. for one hour. The substrate (950 μl) was added to the enzyme-inhibitor solution and incubated at 37° C. for 60 minutes with agitation. Following incubation, samples were centrifuged at 15,000×g (or RPM) for two minutes, the supernatant collected, and absorbance measured at 520 nanometers. All assays were performed in triplicate.

### Fibrinogenolytic Assay

We used the method as described in (58) with modifications. FETUA proteins were incubated with one microgram of MDC-4, MAD-3 or MPO-1 at 37° C. for 60 minutes. Bovine fibrinogen (50 μl at 1 mg/ml, Sigma, catalog #341573) in 50 mM Tris-HCl, pH 7.6, 150 mM NaCl was incubated with the enzyme-inhibitor mixture at 37° C. for 20 minutes. For MPO-1, the incubation time after addition of the substrate was 60 minutes. The samples were then loaded on 8-16% gradient SDS-PAGE gels and analyzed by Coomassie staining.

### Mass Spectrometry

### Enzymatic “In Gel” Digestion

Coomassie R-250 stained gel pieces were de-stained (50% methanol (MeOH), 50% water (H₂O), 100 mM acetic acid (NH₄HCO₃)) completely, dehydrated (50% acetonitrile (ACN), 50% H₂O, 25 mM NH₄HCO₃) for five minutes and then incubated for 30 seconds in 100% ACN. Next, the samples were dried in a Speed-Vac for one minute, reduced in 25 mM DTT (Dithiothreitol) in 25 mM NH₄HCO₃) for 15 minutes at 56° C., alkylated with 55 mM CAA (Chloroacetamide in 25 mM NH₄HCO₃) in darkness at room temperature for 15 minutes, washed once in H₂O, dehydrated (50% ACN, 50% H₂O, 25 mM NH₄HCO₃) for two minutes in then incubated for 30 seconds in 100% ACN. The samples were dried again and rehydrated with 20 μl of trypsin solution with 0.01% ProteaseMAX™ surfactant (10 ng/Trypsin from Promega Corp. in 25 mM NH₄HCO₃, 0.01% w/v of ProteaseMAX™ from Promega Corp.), let stand for 2 minutes at room temperature then an additional 30 μl of overlay solution (25 mM NH₄HCO₃, 0.01% w/v of ProteaseMAX™) was added to keep gel pieces immersed throughout the digestion (three hours at 42° C.). Peptides generated from digestion were transferred to a new tube and acidified with 2.5% TFA (trifluoroacetic Acid) to a 0.3% final concentration. Gel pieces were extracted further with 70% ACN, 29.25% H₂O, 0.75% TFA for ten minutes while vortexing and solutions combined and dried completely in a Speed-Vac (˜15 minutes). Extracted peptides were solubilized in 30 μl of 0.05% TFA. Degraded ProteaseMAX™ was removed via centrifugation (max speed, ten minutes) and the peptides solid-phase extracted (ZipTip® C18 pipette tips from MilliporeSigma) according to the manufacturer's protocol. Peptides were eluted off the C18 SPE column with five microliters of 70% ACN, 30% H₂O, 0.1% TFA, dried to completion, then resolubilized in 30 μl total volume with 0.1% formic acid, and two microliters was loaded on the instrument.

### NanoLC-MS/MS

Peptides were analyzed by NanoLC-MS/MS using the Agilent 1100 nanoflow system (Agilent) connected to a hybrid linear ion trap-orbitrap mass spectrometer (LTQ-Orbitrap Elite™, Thermo Fisher Scientific) equipped with an EASY-Spray™ electrospray source (held at constant 35° C.). Chromatography of peptides prior to mass spectral analysis was accomplished using capillary emitter column (PepMap® C18, 3 μM, 100A, 150×0.075 mm, Thermo Fisher Scientific) onto which two microliters of extracted peptides was automatically loaded. NanoHPLC system delivered solvents A: 0.1% (v/v) formic acid, and B: 99.9% (v/v) acetonitrile, 0.1% (v/v) formic acid at 0.50 μl/min to load the peptides (over a 30 minute period) and 0.3 μl/min to elute peptides directly into the nano-electrospray with gradual gradient from 0% (v/v) B to 30% (v/v) B over 80 minutes and concluded with a five minute fast gradient from 30% (v/v) B to 50% (v/v) B at which time a four minute flash-out from 50-95% (v/v) B took place. Total run time of 150 minutes encompassed column conditioning at 95% B for one minute and equilibration at 100% A for 30 minutes. As peptides eluted from the HPLC-column/electrospray source survey MS scans were acquired in the Orbitrap with a resolution of 120,000 followed by CID-type MS/MS with 2.0 AMU isolation and 10 millisecond activation time with 35% normalized collision energy fragmentation of 30 most intense peptides detected in the MS1 scan from 350 to 1800 m/z; redundancy was limited by dynamic exclusion. Monoisotopic precursor selection and charge state screening were enabled and +1 and undefined charge states were rejected.

### Data Analysis and Cross-Linking Assignment

Raw MS/MS data were converted to mgf file format using MSConvert (ProteoWizard: Open Source Software for Rapid Proteomics Tools Development) for downstream analysis. Resulting mgf files were used to search against user defined C. atrox venom proteome or liver proteome (translated transcriptome) amino acid sequence database with a list of common contaminants (172 total entries) using in-house Mascot search engine 2.7.0 (Matrix Science) with variable Methionine oxidation, Asparagine and Glutamine deamidation plus fixed cysteine Carbamidomethylation. Peptide mass tolerance was set at 10 ppm and fragment mass at 0.8 Da. Protein annotations, significance of identification, and spectral based quantification was done with the help of Scaffold software (version 4.11.0, Proteome Software Inc., Portland, OR). Peptide identifications were accepted if they could be established at greater than 97.0% probability to achieve a False Discovery Rate (FDR) less than 1.0% by the Scaffold Local FDR algorithm. Protein identifications were accepted if they could be established at greater than 61.0% probability to achieve an FDR less than 1.0% and contained at least two identified peptides. Protein probabilities were assigned by the Protein Prophet algorithm (59). Proteins that contained similar peptides and could not be differentiated based on MS/MS analysis alone were grouped to satisfy the principles of parsimony. Proteins sharing significant peptide evidence were grouped into clusters.

### Phylogenetic Analysis

Sequences used in our protein phylogenies were deduced from hypothetical translation of assembled genomic regions or genomic regions from NCBI databases (see full list of sequences and accession numbers in Supplementary Data 4). To construct protein phylogenies, we used MEGA software (v.11.0.11) (60) First, we performed multiple protein sequence alignment using MUSCLE feature (61) in the software. Next, we selected the best substitution model and built maximum likelihood trees using the Jones-Taylor-Thornton substitution model (62). Node support for consensus trees was calculated by Bootstrap analysis on 1000 replicated trees.

Synteny between P. flavoviridis and C. atrox at the fetua loci was determined using the (B)LastZ algorithm (63) in GEvo feature at COGE (https://genomevolution.org/coge/GEvo.pl) (64). Transposable elements were identified using the Repeatmasker web server with the cross_match search engine run in slow mode (A. F. A. Smit, R. Hubley and P. Green, unpublished data. Version: open-4.0.5, RMLib: 20140131, Dfam: 1.3).

The following reference listing is not an indication that any reference is considered material to patentability.
1. P. R. Ehrlich, P. H. Raven, Butterflies and Plants: A study in coevolution. Evolution (N Y) 18, 586-608 (1964).
2. D. H. Janzen, When is it Coevolution? Evolution (NY) 34, 611-612 (1980).
3. B. G. Fry, From genome to “venome”: Molecular origin and evolution of the snake venom proteome inferred from phylogenetic analysis of toxin sequences and related body proteins. Genome Res. 15, 403-420 (2005).
4. J. van Thiel, et al., Convergent evolution of toxin resistance in animals (2022) https:/doi.org/10.1111/brv.12865.
5. V A. Bastos, F. Gomes-Neto, J. Perales, A. G. C. Neves-Ferreira, R. H. Valente, Natural inhibitors of snake venom metalloendopeptidases: History and current challenges. Toxins (Basel) 8 (2016).
6. G. B. Domont, J. Perales, H. Moussatché, Natural anti-snake venom proteins. Toxicon 29, 1183-1194 (1991).
7. J. Perales, A. G. C. Neves-Ferreira, R. H. Valente, G. B. Domont, Natural inhibitors of snake venom hemorrhagic metalloproteinases. Toxicon 45, 1013-1020 (2005).
8. R. R. Martinez, J. C. Perez, E. E. Sanchez, R. Campos, The antihemorrhagic factor of the Mexican ground squirrel, (Spermophilus mexicanus). Toxicon 37, 949-954 (1999).
9. J. E. Biardi, R. G. Coss, D. G. Smith, California ground squirrel (Spermophilus beecheyi) blood sera inhibits crotalid venom proteolytic activity. Toxicon 38, 713-721 (2000).
10. J. E. Biardi, D. C. Chien, R. G. Coss, CALIFORNIA GROUND SQUIRREL (Spermophilus beecheyi) DEFENSES AGAINST RATTLESNAKE VENOM DIGESTIVE AND HEMOSTATIC TOXINS. J Chem Ecol 32 (2006).
11. J. E. Biardi, R. G. Coss, Rock squirrel (Spermophilus variegatus) blood sera affects proteolytic and hemolytic activities of rattlesnake venoms. Toxicon 57, 323-331 (2011).
12. J. E. Biardi, C. Y L. Ho, J. Marcinczyk, K. P. Nambiar, Isolation and identification of a snake venom metalloproteinase inhibitor from California ground squirrel (Spermophilus beecheyi) blood sera. Toxicon 58, 486-493 (2011).
13. M. L. Holding, J. E. Biardi, H. L. Gibbs, Coevolution of venom function and venom resistance in a rattlesnake predator and its squirrel prey. Proceedings of the Royal Society B: Biological Sciences 283, 20152841 (2016).
14. L. Sanz, H. L. Gibbs, S. P. Mackessy, J. J. Calvete, Venom Proteomes of Closely Related Sistrurus Rattlesnakes with Divergent Diets. J Proteome Res 5, 2098-2112 (2006).
15. M. L. Holding, et al., Phylogenetically diverse diets favor more complex venoms in North American pitvipers. Proceedings of the National Academy of Sciences 118, e2015579118 (2021).
16. A. J. Mason, et al., Venom Gene Sequence Diversity and Expression Jointly Shape Diet Adaptation in Pitvipers. Mol Biol Evol 39, msac082 (2022).
17. C. M. Modahl, null Mrinalini, S. Frietze, S. P. Mackessy, Adaptive evolution of distinct prey-specific toxin genes in rear-fanged snake venom. Proceedings of the Royal Society B. Biological Sciences 285, 20181003 (2022).
18. T. Omori-Satoh, A. Ohsaka, Purification and Some Properties of Hemorrhagic Principle I in the Venom of Trimeresurus Flavoviridis. Biochimica et Biophysica Acta (BBA)—Protein Structure 207, 432-444 (1970).
19. M. Ovadia, E. Kochva, Neutralization of Viperidae and Elapidae snake venoms by sera of different animals. Toxicon 15, 541-547 (1977).
20. V. B. Philpot, E. Ezekiel, Y Laseter, R. G. Yaeger, R. L. Stjernholm, Neutralization of crotalid venoms by fractions from snake sera. Toxicon 16, 603-609 (1978).
21. M. Ovadia, Purification and characterization of an antihemorrhagic factor from the serum of the snake Vipera palaestinae. Toxicon 16, 661-672 (1978).
22. A. G. C. Neves-Ferreira, R. H. Valente, G. B. Domont, J. Perales, “Natural Inhibitors of Snake Venom Metallopeptidases” in Toxins and Drug Discovery, L. J. Cruz, S. Luo, P. Gopalakrishnakone, Eds. (Springer Netherlands, 2017), pp. 53-80.
23. E. Kochva, The origin of snakes and evolution of the venom apparatus. Toxicon 25, 65-106 (1987).
24. S. A. Weinstein, P. J. Lafaye, L. A. Smith, Observations on a Venom Neutralizing Fraction Isolated from Serum of the Northern Copperhead, Agkistrodon contortrix mokasen. Copeia 1991, 777-786 (1991).
25. S. Weissenberg, M. Ovadia, E. Kochva, Inhibition of the proteolytic activity of hemorrhagin-e from Crotalus atrox venom by antihemorrhagins from homologous serum. Toxicon 30, 591-597 (1992).
26. S. Weissenberg, M. Ovadia, G. Fleminger, E. Kochva, Antihemorrhagic factors from the blood serum of the western diamondback rattlesnake Crotalus atrox. Toxicon 29, 807-818 (1991).
27. T. Omori-Satoh, S. Sadahiro, A. Ohsaka, R. Murata, Purification and characterization of an antihemorrhagic factor in the serum of Trimeresurus flavoviridis, a crotalid. Biochimica et Biophysica Acta (BBA)—Protein Structure 285, 414-426 (1972).
28. M. Deshimaru, et al., Properties and cDNA cloning of an antihemorrhagic factor (HSF) purified from the serum of Trimeresurus flavoviridis. Toxicon 46, 937-945 (2005).
29. N. Aoki, M. Deshimaru, K. Kihara, S. Terada, Snake fetuin: Isolation and structural analysis of new fetuin family proteins from the sera of venomous snakes. Toxicon 54, 481-490 (2009).
30. R. H. Valente, B. Dragulev, J. Perales, J. W. Fox, G. B. Domont, “BJ46a, a snake venom metalloproteinase inhibitor Isolation, characterization, cloning and insights into its mechanism of action” (2001).
31. M. W. Giorgianni, et al., The origin and diversification of a novel protein family in venomous snakes. PNAS 117, 10911-10920 (2020).
32. D. R. Schield, et al., The origins and evolution of chromosomes, dosage compensation, and mechanisms underlying venom regulation in snakes. Genome Res 29, 590-601 (2019).
33. M. J. Margres, et al., The Tiger Rattlesnake genome reveals a complex genotype underlying a simple venom phenotype. Proceedings of the National Academy of Sciences 118, e2014634118 (2021).
34. J. J. Calvete, E. Fasoli, L. Sanz, E. Boschetti, P. G. Righetti, Exploring the venom proteome of the western diamondback rattlesnake, Crotalus atrox, via snake venomics and combinatorial peptide ligand library approaches. J Proteome Res 8, 3055-3067 (2009).
35. K. E. Robinson, et al., Phenotypic and functional variation in venom and venom resistance of two sympatric rattlesnakes and their prey. J Evol Biol 34, 1447-1465 (2021).
36. D. Melendez-Martinez, et al., Functional Mining of the Crotalus Spp. Venom Protease Repertoire Reveals Potential for Chronic Wound Therapeutics https:/doi.org/10.3390/molecules25153401.
37. N. Aoki, K. Tsutsumi, M. Deshimaru, S. Terada, Properties and cDNA cloning of antihemorrhagic factors in sera of Chinese and Japanese mamushi (Gloydius blomhoffi). Toxicon 51, 251-261 (2008).
38. N. L. Dowell, et al., The Deep Origin and Recent Loss of Venom Toxin Genes in Rattlesnakes. Current Biology 26, 2434-2445 (2016).
39. K. A. Majorek, M. L. Kuhn, M. Chruszcz, W. F. Anderson, W. Minor, Double trouble-Buffer selection and His-tag presence may be responsible for nonreproducibility of biomedical experiments (2014) https:/doi.org/10.1002/pro.2520.
40. P. Ledent, et al., “Unexpected influence of a C-terminal-fused His-tag on the processing of an enzyme and on the kinetic and folding parameters” (1997).
41. N. R. Casewell, S. C. Wagstaff, R. A. Harrison, C. Renjifo, W. Wuster, Domain Loss Facilitates Accelerated Evolution and Neofunctionalization of Duplicate Snake Venom Metalloproteinase Toxin Genes (2011) https:/doi.org/10.1093/molbev/msr091.
42. J. W. Fox, S. M. T. Serrano, Structural considerations of the snake venom metalloproteinases, key members of the M12 reprolysin family of metalloproteinases. Toxicon 45, 969-985 (2005).
43. Y Yamakawa, T. Omori-Satoh, “Primary Structure of the Antihemorrhagic Factor in Serum of the Japanese Habu: A Snake Venom Metalloproteinase Inhibitor with a Double-Headed Cystatin Domain” (1992).
44. R. Marquez, V. Ramirez-Castaneda, A. Amezquita, Does batrachotoxin autoresistance coevolve with toxicity in <em>Phyllobates</em>poison-dart frogs? Evolution (NY) 73, 390-400 (2019).
45. C. C. Hansen, D. R. Nelson, B. L. Møller, D. Werck-Reichhart, Plant cytochrome P450 plasticity and evolution. Mol Plant 14, 1244-1265 (2021).
46. V. A. Bastos, et al., The interaction between the natural metalloendopeptidase inhibitor BJ46a and its target toxin jararhagin analyzed by structural mass spectrometry and molecular modeling. J Proteomics 221, 103761 (2020).
47. A. Silva, G. K. Isbister, Current research into snake antivenoms, their mechanisms of action and applications (2020) https:/doi.org/10.1042/BST20190739.
48. C.-S. Chin, et al., Nonhybrid, finished microbial genome assemblies from long-read SMRT sequencing data. Nat Methods 10, 563-569 (2013).
49. N. L. Dowell, et al., Extremely Divergent Haplotypes in Two Toxin Gene Complexes Encode Alternative Venom Types within Rattlesnake Species. Current Biology 28, 1016-1026.e4 (2018).
50. S. M. Kielbasa, R. Wan, K. Sato, P. Horton, M. C. Frith, Adaptive seeds tame genomic sequence comparison. Genome Res 21, 487-493 (2011).
51. S. F. Altschul, W. Gish, W. Miller, E. W. Myers, D. J. Lipman, Basic local alignment search tool. J Mol Biol 215, 403-410 (1990).
52. H. Li, “Aligning sequence reads, clone sequences and assembly contigs with BWA-MEM” (2013).
53. H. Li, J. Barrett, A statistical framework for SNP calling, mutation discovery, association mapping and population genetical parameter estimation from sequencing data. 27, 2987-2993 (2011).
54. P. Danecek, et al., Twelve years of SAMtools and BCFtools. Gigascience 10, giab008 (2021).
55. B. J. Haas, et al., De novo transcript sequence reconstruction from RNA-seq using the Trinity platform for reference generation and analysis. Nat Protoc 8, 1494-1512 (2013).
56. Williams J. A., J. A. Langeland, B. S. Thalley, J. B. Skeath, and S. B. Carroll, Production and purification of polyclonal antibodies against proteins expressed in E. coli. In DNA Cloning: Expression Systems. (1995).
57. H. F. Williams, et al., Mechanisms underpinning the permanent muscle damage induced by snake venom metalloprotease. PLoS Negl Trop Dis 13 (2019).
58. Y P. Han, X. Y Lu, X. F. Wang, J. Xu, Isolation and characterization of a novel P-II class snake venom metalloproteinase from Trimeresurus stejnegeri. Toxicon 49, 889-898 (2007).
59. A. I. Nesvizhskii, A. Keller, E. Kolker, R. Aebersold, A Statistical Model for Identifying Proteins by Tandem Mass Spectrometry. Anal Chem 75, 4646-4658 (2003).
60. S. Kumar, G. Stecher, M. Li, C. Knyaz, K. Tamura, MEGA X: Molecular evolutionary genetics analysis across computing platforms. Mol Biol Evol 35, 1547-1549 (2018).
61. R. C. Edgar, MUSCLE: multiple sequence alignment with high accuracy and high throughput. Nucleic Acids Res 32, 1792-1797 (2004).
62. B. G. Hall, Building Phylogenetic Trees from Molecular Data with MEGA https:/doi.org/10.1093/molbev/mst012.
63. S. Schwartz, et al., Human-Mouse Alignments with BLASTZ. Genome Res 13, 103-107 (2003).
64. E. Lyons, M. Freeling, How to usefully compare homologous plant genes and chromosomes as DNA sequences. The Plant Journal 53, 661-673 (2008).

### Supplemental Data

S. Data. 1: (A) List of primers used to recover cDNA clones from C. atrox liver mRNA (B) Primers used to screen BAC libraries for FETUA loci in C. adamanteus and C. atrox

A. cDNA-PCRs

| Target | 5′ primer | 3′primer | Amplicon |
|---|---|---|---|
| FETUA-5 | ATGAATTCCCTGGTAGCTCTCGTG (SEQ ID NO: 9) | oligo-dT | full length |
| FETUA-3 | ATGAATTCCCTGGTAGCTCTCGTG (SEQ ID NO: 9) | oligo-dT | full length |
| FETUA-1 | ATGAATTCCCTGGTAGCTCTCGTG (SEQ ID NO: 9) | oligo-dT | full length |
| FETUA- | GAGTCTTCCTTCTTTCCAACTAGTG | TAAAATACCGTCGTAAACCTCTCCG | TSS-227 bp |
| 4a | (SEQ ID NO: 10) | (SEQ ID NO: 11) | |
| FETUA- | CCGGAGAGGTTTACGACGGT | TCTCAATGCTCTCTAGTATGGCTGG | 202 bp- |
| 4a | (SEQ ID NO: 12) | (SEQ ID NO: 13) | 3′UTR |
| FETUA- | GAGTCTTCCTTCTTTCCAACTAGTG | TTTAAGGTAGACTGCCACCAAATC | TSS-160 bp |
| 2a | (SEQ ID NO: 10) | (SEQ ID NO: 14) | |
| FETUA- | TCTCAAGTGAGGGGGGATTTAGAATGT | oligo-dT | 60 bp-stop |
| 2a | (SEQ ID NO: 15) | | |

B . BAC Library screen

| Target | 5′ primer | 3′primer | Amplicon |
|---|---|---|---|
| FETUA Ex 5-7 | GTTTGCTATTGTGGAGGTTAACTGCACT | CACCACACATTCATCAGTACTGTTGC | ~2 Kb |
| | (SEQ ID NO: 16) | (SEQ ID NO: 17) | |

S. Data. 2: Identification of the purified SVMP proteins from C. atrox venom via Mass Spectrometry by LC-MS/MS. Purified fraction (A) MDC-4, (B) MAD-3a/b, and (C) MPO-1 higher ˜20 kDa and lower ˜13 kDa bands). The highest number of peptides within each sample are shown in bold.

(A) MDC-4

| | Exclusive | Total Spectral | Total Unique |
|---|---|---|---|
| Proteins | Spectrum Count | Count | Peptide Count |
| atx_{—}mdc-4 | 1844 | 1995 | 49 |
| atx_mdc-5a | 3 | 55 | 9 |
| atx_mdc-5b | 4 | 56 | 11 |
| atx_mdc-2 | 1 | 46 | 4 |
| atx_mad-3a | 3 | 11 | 9 |
| atx_mad-3b | 1 | 8 | 7 |
| atx_mdc-8a | 4 | 5 | 3 |
| atx_mdc-8c | 5 | 6 | 3 |
| atx_mad-2b | 1 | 4 | 4 |
| atx_mpo-1 | 0 | 2 | 2 |
| atx_mdc-3b | 1 | 4 | 4 |
| atx_mdc-3a | 1 | 4 | 4 |
| atx_gA1 | 15 | 15 | 6 |
| atx_mdc-6a | 4 | 13 | 8 |
| OXLA_CROAT_Laao | 14 | 14 | 10 |
| VSP1_CROAT_svsp_catroxase-I | 16 | 16 | 7 |
| atx_mdc-6d | 9 | 10 | 6 |
| atx_mad-5b | 6 | 8 | 6 |
| VSPA1_CROAT_Alpha-fibrinogenase-A1 | 9 | 9 | 1 |
| VSP2_CROAT_svsp_catroxase-2 | 7 | 7 | 6 |
| VSPC1_CROAT_svsp_catroxbin-1 | 2 | 2 | 1 |
| TXVE_CROAT_sVEGF_Cratrin | 1 | 1 | 1 |
| atx_mdc-6e | 1 | 1 | 1 |

(B) MAD-3

| | Exclusive | Total Spectral | Total Unique |
|---|---|---|---|
| Protein | Spectrum Count | Count | Peptide Count |
| atx_mpo-1 | 26 | 29 | 13 |
| atx_{—}mad-3a | 66 | 248 | 11 |
| atx_{—}mad-3b | 48 | 230 | 12 |
| atx_mad-6 | 12 | 12 | 2 |

(C) MPO-1

| | Exclusive | Total Spectral | Total Unique |
|---|---|---|---|
| Protein | Spectrum Count | Count | Peptide Count |
| atx_mpo-1 | 501 | 613 | 25 |
| atx_mad-5a | 1 | 1 | 1 |

S. Data. 3(A): Assignment of the FETUA-3 Affinity purified total eluate fractions of C. atrox Venom to SVMP genes via Mass Spectrometry by LC-MS/MS

| | Protein | | |
|---|---|---|---|
| Protein | identification | | Number of |
| name | probability | Peptide sequence | Spectra |
| atx_mad-1b | 97.90% | VTTGSQCAEGLCCDQCK (SEQ ID NO: 18) | 1 |
| atx_mad-3a | 100.00% | RHDNAQLLTSIDFDGPTIGLAYIGSICDPK (SEQ ID NO: 19) | 3 |
| | 100.00% | RHDNAQLLTSIDFDGPTIGLAYIGSICDPKR (SEQ ID NO: 20) | 1 |
| | 100.00% | RSTGVVQDHSEINLR (SEQ ID NO: 21) | 19 |
| | 100.00% | STGVVQDHSEINLR (SEQ ID NO: 22) | 15 |
| | 100.00% | YFSDCSYIQCWNFIMNQKPQCILK (SEQ ID NO: 23) | 6 |
| | 100.00% | YVELFIVVDHGMFTK (SEQ ID NO: 24) | 25 |
| atx_mad-3b | 100.00% | DGICDPKR (SEQ ID NO: 25) | 7 |
| | 100.00% | ISHDNAQLLTSTDFNR (SEQ ID NO: 26) | 6 |
| | 100.00% | QAIGLAYR (SEQ ID NO: 27) | 3 |
| | 100.00% | QAIGLAYRDGICDPK (SEQ ID NO: 28) | 1 |
| | 100.00% | RSTGVVQDHSK (SEQ ID NO: 29) | 2 |
| | 100.00% | STGVVQDHSK (SEQ ID NO: 30) | 6 |
| | 100.00% | VSMVDRNDDTCTGQSADCPR (SEQ ID NO: 31) | 2 |
| | 100.00% | YFSDCSYIQCWDFIMNQNPQCILK (SEQ ID NO: 32) | 15 |
| | 100.00% | YVELFIVVDHGMYTK (SEQ ID NO: 33) | 43 |
| atx_mad-4a | 100.00% | AWVYEIFNTINEIFQR (SEQ ID NO: 34) | 1 |
| | 100.00% | SHDNAQLLTSIDLDGPTIGLAYIGGICDPK (SEQ ID NO: 35) | 3 |
| atx_mad-4b | 100.00% | AWVYEIVNTINEIFQR (SEQ ID NO: 36) | 2 |
| | 100.00% | IIVQSSADITLDLFGTWR (SEQ ID NO: 37) | 4 |
| | 100.00% | NLCCDAATCK (SEQ ID NO: 38) | 1 |
| | 100.00% | SHDNAQLLTSINFDGQTIGWAYIGGICDSK (SEQ ID NO: 39) | 35 |
| | 100.00% | SHDNAQLLTSINFDGQTIGWAYIGGICDSKR (SEQ ID NO: 40) | 17 |
| | 100.00% | STGVVQDFSPINFLVAITMAHEMGHNLGMTHDENYCSCGGFACIMSPVISPQ | 0 |
| | | PSK (SEQ ID NO: 41) | |
| atx_mad-5a | 100.00% | AYTGSMCDPR (SEQ ID NO: 42) | 4 |
| | 100.00% | AYTGSMCDPRK (SEQ ID NO: 43) | 1 |
| | 100.00% | ISHDNAQLLTAINFQENIIGR (SEQ ID NO: 44) | 21 |
| | 100.00% | ISHDNAQLLTAINFQENIIGRAYTGSMCDPR (SEQ ID NO: 45) | 1 |
| | 100.00% | IWVHEIVNTINVFYR (SEQ ID NO: 46) | 6 |
| | 100.00% | YIELVIVADHR (SEQ ID NO: 47) | 29 |
| | 100.00% | YNSNLNTIR (SEQ ID NO: 48) | 9 |
| | 100.00% | YNSNLNTIRIWVHEIVNTINVFYR (SEQ ID NO: 49) | 1 |
| atx_mdc-2 | 100.00% | AAKDECDMADVCTGR (SEQ ID NO: 50) | 1 |
| | 100.00% | ACSNGQCVDVTTPY (SEQ ID NO: 51) | 2 |
| | 100.00% | CPIMADQCIALFGPGATVSQDACFQFNR (SEQ ID NO: 52) | 4 |
| | 100.00% | DECDMADVCTGR (SEQ ID NO: 53) | 2 |
| | 100.00% | DHREFLIK (SEQ ID NO: 54) | 1 |
| | 100.00% | DTCTCGTRPCVMAGALSCEASFLFSDCSQK (SEQ ID NO: 55) | 1 |
| | 100.00% | EGNHYGYCR (SEQ ID NO: 56) | 1 |
| | 100.00% | IACEPQDVK (SEQ ID NO: 57) | 1 |
| | 100.00% | LRQGAQCAEGLCCDQCR (SEQ ID NO: 58) | 1 |
| | 100.00% | MYDIVNVITPIYHR (SEQ ID NO: 59) | 1 |
| | 100.00% | NMPQCILK (SEQ ID NO: 60) | 1 |
| | 100.00% | NNNGYCYNGK (SEQ ID NO: 61) | 2 |
| | 100.00% | NPCNIYYSPNDEDK (SEQ ID NO: 62) | 1 |
| | 100.00% | QGAQCAEGLCCDQCR (SEQ ID NO: 63) | 2 |
| | 100.00% | SHDNAQLLTGINFNGPTAGLGYLGGICNTMYSAGIVQDHSK (SEQ ID NO: | 5 |
| | | 64) | |
| | 100.00% | TCRDPCCDATTCK (SEQ ID NO: 65) | 2 |
| | 100.00% | TDVVSPAVCGNYFVEVGEECDCGSPR (SEQ ID NO: 66) | 1 |
| atx_mdc-3a | 99.20% | LHSWVECESGECCDQCR (SEQ ID NO: 67) | 1 |
| atx_mdc-4 | 100.00% | AMVTKNNGDLDK (SEQ ID NO: 68) | 2 |
| | 100.00% | ASMSECDPAEHCTGQSSECPADVFHK (SEQ ID NO: 69) | 35 |
| | 100.00% | DNSPGQNNPCK (SEQ ID NO: 70) | 45 |
| | 100.00% | ENGNKIPCAPEDVK (SEQ ID NO: 71) | 14 |
| | 100.00% | FVELFLVVDK (SEQ ID NO: 72) | 35 |
| | 100.00% | GNYYGYCR (SEQ ID NO: 73) | 10 |
| | 100.00% | GNYYGYCRK (SEQ ID NO: 74) | 12 |
| | 100.00% | HDNAQLLTAIDLDR (SEQ ID NO: 75) | 55 |
| | 100.00% | HDNAQLLTAIDLDRVIGLAYVGSMCHPK (SEQ ID NO: 76) | 11 |
| | 100.00% | HDNAQLLTAIDLDRVIGLAYVGSMCHPKR (SEQ ID NO: 77) | 0 |
| | 100.00% | ITVKPEAGYTLNAFGEWR (SEQ ID NO: 78) | 101 |
| | 100.00% | ITVKPEAGYTLNAFGEWRK (SEQ ID NO: 79) | 21 |
| | 100.00% | ITVKPEAGYTLNAFGEWRKTDLLTR (SEQ ID NO: 80) | 1 |
| | 100.00% | KENGNKIPCAPEDVK (SEQ ID NO: 81) | 4 |
| | 100.00% | KHDNAQLLTAIDLDR (SEQ ID NO: 82) | 75 |
| | 100.00% | KHDNAQLLTAIDLDRVIGLAYVGSMCHPK (SEQ ID NO: 83) | 8 |
| | 100.00% | KKHDNAQLLTAIDLDR (SEQ ID NO: 84) | 3 |
| | 100.00% | KTDLLTRK (SEQ ID NO: 85) | 5 |
| | 100.00% | LYCKDNSPGQNNPCK (SEQ ID NO: 86) | 1 |
| | 100.00% | MFYSNEDEHK (SEQ ID NO: 87) | 50 |
| | 100.00% | MYEIVNTVNEIYR (SEQ ID NO: 88) | 124 |
| | 100.00% | MYEIVNTVNEIYRYMYIHVALVGLEIWSNEDK (SEQ ID NO: 89) | 6 |
| | 100.00% | MYEIVNTVNEIYRYMYIHVALVGLEIWSNEDKITVKPEAGYTLNAFGEWR | 1 |
| | | (SEQ ID NO: 90) | |
| | 100.00% | NGQPCLDNYGYCYNGNCPIMYHQCYDLFGADVYEAEDSCFER (SEQ ID | 136 |
| | | NO: 91) | |
| | 100.00% | NQKGNYYGYCR (SEQ ID NO: 92) | 1 |
| | 100.00% | TRMYEIVNTVNEIYR (SEQ ID NO: 93) | 3 |
| | 100.00% | TRMYEIVNTVNEIYRYMYIHVALVGLEIWSNEDK (SEQ ID NO: 94) | 1 |
| | 100.00% | VCSNGHCVDVATAY (SEQ ID NO: 95) | 32 |
| | 100.00% | VIGLAYVGSMCHPK (SEQ ID NO: 96) | 91 |
| | 100.00% | VIGLAYVGSMCHPKR (SEQ ID NO: 97) | 1 |
| | 100.00% | YMYIHVALVGLEIWSNEDK (SEQ ID NO: 98) | 102 |
| | 100.00% | YMYIHVALVGLEIWSNEDKITVKPEAGYTLNAFGEWR (SEQ ID NO: 99) | 4 |
| | 100.00% | YNPFRFVELFLVVDKAMVTK (SEQ ID NO: 100) | 1 |
| at_mdc-5a | 100.00% | AFDSNEDDHK (SEQ ID NO: 101) | 1 |
| | 100.00% | AHVASMCEPK (SEQ ID NO: 102) | 1 |
| | 100.00% | GDDDGYCR (SEQ ID NO: 103) | 4 |
| | 100.00% | GDDDGYCRK (SEQ ID NO: 104) | 2 |
| | 100.00% | HDNAQLLTAIDFSGSTIGK (SEQ ID NO: 105) | 1 |
| | 100.00% | RSECDIAESCTGQSDDCPTDDFHR (SEQ ID NO: 106) | 3 |
| | 100.00% | SECDIAESCTGQSDDCPTDDFHR (SEQ ID NO: 107) | 1 |
| | 100.00% | VCSNGHCVDVASAY (SEQ ID NO: 108) | 4 |
| atx_mdc-5b | 100.00% | AYIASMCDPK (SEQ ID NO: 109) | 5 |
| | 100.00% | FCTNGHCVDVATAY (SEQ ID NO: 110) | 2 |
| | 100.00% | GDDYGYCRK (SEQ ID NO: 111) | 1 |
| | 100.00% | LYCNDNSPGQNNPCK (SEQ ID NO: 112) | 1 |
| | 100.00% | RSECDIAESCTGQSADCPMDDFHK (SEQ ID NO: 113) | 2 |
| atx_mdc-6a | 100.00% | LFCNVNDFLCR (SEQ ID NO: 114) | 7 |
| | 100.00% | NRQCVDVTTAYK (SEQ ID NO: 115) | 1 |
| | 100.00% | QCVDVTTAYK (SEQ ID NO: 116) | 1 |
| | 100.00% | RYVELVIVADHR (SEQ ID NO: 117) | 12 |
| | 100.00% | YVELVIVADHR (SEQ ID NO: 118) | 1 |
| atx_mdc-6b | 100.00% | EQPSYEFSDCSQNQYLR (SEQ ID NO: 119) | 1 |
| | 100.00% | YSADPDYGMVNHGTK (SEQ ID NO: 120) | 1 |
| atx_mdc-6c | 100.00% | LFCEVNNFPCR (SEQ ID NO: 121) | 1 |
| atx_mdc-6d | 100.00% | ENGVNIPCSYEDVK (SEQ ID NO: 122) | 2 |
| | 100.00% | FALVGLEIWSNGDK (SEQ ID NO: 123) | 1 |
| | 100.00% | ITVQSSAYNTLDSFEEWRETDLLTR (SEQ ID NO: 124) | 2 |
| | 100.00% | LFCEFNNFPCQYK (SEQ ID NO: 125) | 2 |
| | 100.00% | RHDNAQLLTAIDEDGR (SEQ ID NO: 126) | 2 |
| | 100.00% | SECDIAESCTGQSADCPTDDFHR (SEQ ID NO: 127) | 3 |
| | 100.00% | TLGLASVSSMCNQK (SEQ ID NO: 128) | 1 |
| | 100.00% | YSEDLDYGMVDHGTK (SEQ ID NO: 129) | 2 |
| atx_mdc-6e | 99.90% | DDCDMAESCTGR (SEQ ID NO: 130) | 1 |
| atx_mdc-7 | 100.00% | DDCDMADLCTGQSAECPTDR (SEQ ID NO: 131) | 4 |
| | 100.00% | DEDIGMVLPGTK (SEQ ID NO: 132) | 2 |
| | 100.00% | GSSYGYCR (SEQ ID NO: 133) | 1 |
| | 100.00% | GSSYGYCRK (SEQ ID NO: 134) | 1 |
| | 100.00% | GSVGVIQEHSTINLLMAVTMAHEMGHNLGMDHDIK (SEQ ID NO: 135) | 2 |
| | 100.00% | IPCPPQDVK (SEQ ID NO: 136) | 1 |
| | 100.00% | IYEIVNTMNEIYIPLNIHVALVR (SEQ ID NO: 137) | 10 |
| | 100.00% | KYVEFVVVLDHGMYTK (SEQ ID NO: 138) | 4 |
| | 100.00% | LFCFPNKPGEK (SEQ ID NO: 139) | 2 |
| | 100.00% | LTPGSQCADGVCCDQCR (SEQ ID NO: 140) | 4 |
| | 100.00% | NGHPCLNNK (SEQ ID NO: 141) | 9 |
| | 100.00% | NQCISFFGPSATVAK (SEQ ID NO: 142) | 3 |
| | 100.00% | NVCNVIYMSR (SEQ ID NO: 143) | 3 |
| | 100.00% | NVCNVIYMSRDEDIGMVLPGTK (SEQ ID NO: 144) | 5 |
| | 100.00% | RPQCILNEPLR (SEQ ID NO: 145) | 2 |
| | 100.00% | RPQCILNEPLRTDIVSPPVCGNELLEVGEECDCGSPGNCQNPCCNATTCK | 2 |
| | | (SEQ ID NO: 146) | |
| | 100.00% | SHDNAQLLTTIDFDGDTVGLAYVR (SEQ ID NO: 147) | 8 |
| | 100.00% | VCNSNGQCVDVNR (SEQ ID NO: 148) | 10 |
| | 100.00% | VCNSNGQCVDVNRAY (SEQ ID NO: 149) | 2 |
| | 100.00% | YCTCGAPSCIMADTLSHQPSK (SEQ ID NO: 150) | 3 |
| | 100.00% | YVEFVVVLDHGMYTK (SEQ ID NO: 151) | 7 |
| atx_mdc-8b | 100.00% | SECDIAESCTGR (SEQ ID NO: 152) | 1 |
| atx_mdc-8c | 100.00% | CPTLDHQCISFFGSNAAVAPDVCFDFNLK (SEQ ID NO: 153) | 1 |
| | 100.00% | FSDCSRDEHWR (SEQ ID NO: 154) | 2 |
| | 100.00% | GEGNFYCR (SEQ ID NO: 155) | 1 |
| | 100.00% | HDNAQLLTAIDLDGPTIGLAR (SEQ ID NO: 156) | 1 |
| | 100.00% | IFPCAPQDK (SEQ ID NO: 157) | 1 |
| | 100.00% | IFPCAPQDKK (SEQ ID NO: 158) | 2 |
| | 100.00% | LFCVLGPTGNTISCQSTSSQSDLDIGMVDLGTK (SEQ ID NO: 159) | 3 |
| | 100.00% | LRPGTQCEDGECCEQCQFR (SEQ ID NO: 160) | 1 |
| | 100.00% | NGQPCLNNNGYCYNGK (SEQ ID NO: 161) | 8 |
| | 100.00% | RHDNAQLLTAIDLDGPTIGLAR (SEQ ID NO: 162) | 1 |
| | 100.00% | RHDNAQLLTAIDLDGPTIGLARVGSMCDPK (SEQ ID NO: 163) | 0 |
| | 100.00% | RSTGIVQDHSK (SEQ ID NO: 164) | 1 |
| | 100.00% | SECDIAESCTGQSADCPTDNFQR (SEQ ID NO: 165) | 3 |
| | 100.00% | STGIVQDHSK (SEQ ID NO: 166) | 4 |
| atx_mpf-1a | 100.00% | LIFELGYR (SEQ ID NO: 167) | 1 |
| | 100.00% | LKPGTQCEDGVCCDR (SEQ ID NO: 168) | 1 |
| atx_mpo-1 | 100.00% | AYTSSMCNPR (SEQ ID NO: 169) | 29 |
| | 100.00% | DHSPINLLVGVTMAHELGHNLGMNHDGDK (SEQ ID NO: 170) | 51 |
| | 100.00% | GASLCIMRPGLTPGR (SEQ ID NO: 171) | 50 |
| | 100.00% | ITTNPSVEDHCYYR (SEQ ID NO: 172) | 2 |
| | 100.00% | KRVHELVNTINGFYR (SEQ ID NO: 173) | 3 |
| | 100.00% | KSHDHAQLLTAINFEGK (SEQ ID NO: 174) | 2 |
| | 100.00% | NTLNSFGEWR (SEQ ID NO: 175) | 27 |
| | 100.00% | NTLNSFGEWREADLLR (SEQ ID NO: 176) | 2 |
| | 100.00% | RVHELVNTINGFYR (SEQ ID NO: 177) | 17 |
| | 100.00% | SHDHAQLLTAINFEGK (SEQ ID NO: 178) | 112 |
| | 100.00% | SHDHAQLLTAINFEGKIIGR (SEQ ID NO: 179) | 1 |
| | 100.00% | SHDHAQLLTAINFEGKIIGRAYTSSMCNPR (SEQ ID NO: 180) | 3 |
| | 100.00% | SLNIDVSLTDLEIWSDQDFITVQSSAK (SEQ ID NO: 181) | 50 |
| | 100.00% | SVGIVKDHSPINLLVGVTMAHELGHNLGMNHDGDK (SEQ ID NO: 182) | 0 |
| | 100.00% | VFMKYNSDLNIIR (SEQ ID NO: 183) | 1 |
| | 100.00% | VHELVNTINGFYR (SEQ ID NO: 184) | 45 |
| | 100.00% | YIELVVVADHRVFMK (SEQ ID NO: 185) | 3 |
| | 100.00% | YNSDLNIIR (SEQ ID NO: 186) | 20 |
| | 100.00% | YNSDLNIIRK (SEQ ID NO: 187) | 10 |

S. Data. 3(B): Assignment of the FETUA-3 Affinity purified fraction (gel excised, upper band) of C. atrox Venom to SVMP genes via Mass Spectrometry by LC-MS/MS

| | Protein | | | Number |
|---|---|---|---|---|
| Protein | identification | | | of |
| name | probability | SL# | Peptide sequence | Spectra |
| atx_mad-3b | 98% | | QAIGLAYR (SEQ ID NO: 27) | 1 |
| atx_mad-5a | 99% | | YNSNLNTIR (SEQ ID NO: 48) | 1 |
| atx_mdc-2 | 99% | | EGNHYGYCR (SEQ ID NO: 56) | 1 |
| atx_mdc-3a | 98% | | VTLDSFGNWR (SEQ ID NO: 188) | 1 |
| atx_mdc-4 | 100% | | AMVTKNNGDLDK (SEQ ID NO: 68) | 3 |
| | 100% | | AMVTKNNGDLDKIK (SEQ ID NO: 189) | 2 |
| | 100% | | ASMSECDPAEHCTGQSSECPADVFHK (SEQ ID NO: 69) | 11 |
| | 100% | | CADGKVCSNGHCVDVATAY (SEQ ID NO: 190) | 4 |
| | 100% | | DNSPGQNNPCK (SEQ ID NO: 70) | 38 |
| | 100% | | DNSPGQNNPCKMFYSNEDEHK (SEQ ID NO: 191) | 1 |
| | 100% | | ENGNKIPCAPEDVK (SEQ ID NO: 71) | 7 |
| | 100% | | ENGNKIPCAPEDVKCGR (SEQ ID NO: 192) | 3 |
| | 100% | | FSKSGTECR (SEQ ID NO: 193) | 3 |
| | 100% | | FVELFLVVDK (SEQ ID NO: 72) | 2 |
| | 100% | | FVELFLVVDKAMVTK (SEQ ID NO: 194) | 1 |
| | 100% | | GNYYGYCR (SEQ ID NO: 73) | 23 |
| | 100% | | GNYYGYCRK (SEQ ID NO: 74) | 4 |
| | 100% | | HDNAQLLTAIDLDR (SEQ ID NO: 75) | 8 |
| | 100% | | HDNAQLLTAIDLDRVIGLAYVGSMCHPK (SEQ ID NO: 76) | 6 |
| | 100% | | HDNAQLLTAIDLDRVIGLAYVGSMCHPKR (SEQ ID NO: 77) | 0 |
| | 100% | | ITVKPEAGYTLNAFGEWR (SEQ ID NO: 78) | 9 |
| | 100% | | ITVKPEAGYTLNAFGEWRK (SEQ ID NO: 79) | 3 |
| | 100% | | KENGNKIPCAPEDVK (SEQ ID NO: 81) | 21 |
| | 100% | | KHDNAQLLTAIDLDR (SEQ ID NO: 82) | 14 |
| | 100% | | KHDNAQLLTAIDLDRVIGLAYVGSMCHPK (SEQ ID NO: 83) | 2 |
| | 100% | | KKHDNAQLLTAIDLDR (SEQ ID NO: 84) | 35 |
| | 100% | | KTDLLTRK (SEQ ID NO: 85) | 2 |
| | 100% | | LKSGSQCGHGDCCEQCKFSK (SEQ ID NO: 195) | 1 |
| | 100% | | LYCKDNSPGQNNPCK (SEQ ID NO: 86) | 14 |
| | 100% | | MFYSNEDEHK (SEQ ID NO: 87) | 9 |
| | 100% | | MFYSNEDEHKGMVLPGTK (SEQ ID NO: 196) | 15 |
| | 100% | | MYEIVNTVNEIYR (SEQ ID NO: 88) | 31 |
| | 100% | | NGQPCLDNYGYCYNGNCPIMYHQCYDLFGADVYEAEDSCFER | |
| | | | (SEQ ID NO: 91) | 2 |
| | 100% | | NQKGNYYGYCR (SEQ ID NO: 92) | 8 |
| | 100% | | SGSQCGHGDCCEQCKFSK (SEQ ID NO: 197) | 2 |
| | 100% | | SGSQCGHGDCCEQCKFSKSGTECR (SEQ ID NO: 198) | 1 |
| | 100% | | SGTECRASMSECDPAEHCTGQSSECPADVFHK (SEQ ID NO: 199) | 0 |
| | 100% | | TRMYEIVNTVNEIYR (SEQ ID NO: 93) | 4 |
| | 100% | | VCSNGHCVDVATAY (SEQ ID NO: 95) | 7 |
| | 100% | | VIGLAYVGSMCHPK (SEQ ID NO: 96) | 13 |
| | 100% | | VIGLAYVGSMCHPKR (SEQ ID NO: 97) | 2 |
| | 100% | | YMYIHVALVGLEIWSNEDK (SEQ ID NO: 98) | 3 |
| atx_mdc-5a | 100% | | NQKGDDDGYCR (SEQ ID NO: 200) | 2 |
| | 100% | | NQKGDDDGYCRK (SEQ ID NO: 201) | 1 |
| | 100% | | RSECDIAESCTGQSDDCPTDDFHR (SEQ ID NO: 106) | 2 |
| | 100% | | SECDIAESCTGQSDDCPTDDFHR (SEQ ID NO: 107) | 1 |
| | 100% | | VCSNGHCVDVASAY (SEQ ID NO: 108) | 1 |
| atx_mdc-5b | 100% | | FCTNGHCVDVATAY (SEQ ID NO: 110) | 1 |
| atx_mdc-6a | 100% | | LFCNVNDFLCR (SEQ ID NO: 114) | 1 |
| | 100% | | QCVDVTTAYK (SEQ ID NO: 116) | 1 |
| atx_mdc-6d | 100% | | ENGVNIPCSYEDVK (SEQ ID NO: 122) | 1 |
| | 100% | | KRHDNAQLLTAIDFDGR (SEQ ID NO: 202) | 1 |
| | 100% | | SECDIAESCTGQSADCPTDDFHR (SEQ ID NO: 127) | 1 |
| | 100% | | TLGLASVSSMCNQK (SEQ ID NO: 128) | 1 |
| | 100% | | YSEDLDYGMVDHGTK (SEQ ID NO: 129) | 1 |
| atx_mdc-7 | 100% | | GKVCNSNGQCVDVNR (SEQ ID NO: 203) | 1 |
| | 100% | | LFCFPNKPGEK (SEQ ID NO: 139) | 1 |
| | 100% | | LTPGSQCADGVCCDQCR (SEQ ID NO: 140) | 1 |
| | 100% | | NQCISFFGPSATVAK (SEQ ID NO: 142) | 1 |
| | 100% | | RPQCILNEPLR (SEQ ID NO: 145) | 1 |
| | 100% | | VCNSNGQCVDVNR (SEQ ID NO: 148) | 1 |
| | 100% | | YKDDLDKIK (SEQ ID NO: 204) | 1 |
| atx_mdc-8c | 100% | | IFPCAPQDKK (SEQ ID NO: 158) | 2 |
| | 100% | | NGQPCLNNNGYCYNGK (SEQ ID NO: 161) | 8 |
| | 100% | | RSTGIVQDHSK (SEQ ID NO: 164) | 1 |
| | 100% | | SECDIAESCTGQSADCPTDNFQR (SEQ ID NO: 165) | 1 |
| | 100% | | STGIVQDHSK (SEQ ID NO: 166) | 4 |
| atx_mpf-1a | 98% | | ASQLNFTPEQR (SEQ ID NO: 205) | 1 |
| atx_mpo-1 | 100% | | GASLCIMRPGLTPGR (SEQ ID NO: 171) | 1 |
| | 100% | | NTLNSFGEWR (SEQ ID NO: 175) | 1 |
| | 100% | | SLNIDVSLTDLEIWSDQDFITVQSSAK (SEQ ID NO: 181) | 5 |
| | 100% | | VHELVNTINGFYR (SEQ ID NO: 184) | 2 |
| | 100% | | YNSDLNIIRK (SEQ ID NO: 187) | 1 |

S. Data. 3(C): Assignment of the FETUA-3 Affinity purified fraction (gel excised, lower band) of C. atrox Venom to SVMP genes via Mass Spectrometry by LC-MS/MS

| | Protein | | | |
|---|---|---|---|---|
| Protein | identification | | | Number of |
| name | probability | SL# | Peptide sequence | Spectra |
| atx_mad-3a | 100.00% | | RSTGVVQDHSEINLR (SEQ ID NO: 21) | 2 |
| | 100.00% | | STGVVQDHSEINLR (SEQ ID NO: 22) | 2 |
| atx_mad-3b | 100.00% | | DGICDPKR (SEQ ID NO: 25) | 7 |
| | 100.00% | | ISHDNAQLLTSTDFNR (SEQ ID NO: 26) | 4 |
| | 100.00% | | QAIGLAYRDGICDPK (SEQ ID NO: 28) | 1 |
| | 100.00% | | RSTGVVQDHSK (SEQ ID NO: 29) | 1 |
| | 100.00% | | STGVVQDHSK (SEQ ID NO: 30) | 2 |
| | 100.00% | | YFSDCSYIQCWDFIMNQNPQCILK (SEQ ID NO: 32) | 1 |
| | 100.00% | | YVELFIVVDHGMYTK (SEQ ID NO: 33) | 2 |
| atx_mad-5a | 100.00% | | AYTGSMCDPR (SEQ ID NO: 42) | 1 |
| | 100.00% | | AYTGSMCDPRK (SEQ ID NO: 43) | 2 |
| | 100.00% | | ISHDNAQLLTAINFQENIIGR (SEQ ID NO: 44) | 3 |
| | 100.00% | | YIELVIVADHR (SEQ ID NO: 47) | 2 |
| | 100.00% | | YNSNLNTIR (SEQ ID NO: 48) | 1 |
| atx_mdc-4 | 100.00% | | ASMSECDPAEHCTGQSSECPADVFHK (SEQ ID NO: 69) | 3 |
| | 100.00% | | DNSPGQNNPCK (SEQ ID NO: 70) | 5 |
| | 100.00% | | ENGNKIPCAPEDVK (SEQ ID NO: 71) | 1 |
| | 100.00% | | FVELFLVVDK (SEQ ID NO: 72) | 1 |
| | 100.00% | | GNYYGYCR (SEQ ID NO: 73) | 1 |
| | 100.00% | | HDNAQLLTAIDLDR (SEQ ID NO: 75) | 1 |
| | 100.00% | | ITVKPEAGYTLNAFGEWR (SEQ ID NO: 78) | 1 |
| | 100.00% | | KENGNKIPCAPEDVK (SEQ ID NO: 81) | 3 |
| | 100.00% | | KHDNAQLLTAIDLDR (SEQ ID NO: 82) | 1 |
| | 100.00% | | KKHDNAQLLTAIDLDR (SEQ ID NO: 84) | 1 |
| | 100.00% | | LYCKDNSPGQNNPCK (SEQ ID NO: 86) | 2 |
| | 100.00% | | MYEIVNTVNEIYR (SEQ ID NO: 88) | 1 |
| | 100.00% | | VCSNGHCVDVATAY (SEQ ID NO: 95) | 3 |
| | 100.00% | | VIGLAYVGSMCHPK (SEQ ID NO: 96) | 2 |
| | 100.00% | | YMYIHVALVGLEIWSNEDK (SEQ ID NO: 98) | 1 |
| atx_mdc-6a | 98.20% | | YVELVIVADHR (SEQ ID NO: 118) | 1 |
| atx_mpo-1 | 100.00% | | AYTSSMCNPR (SEQ ID NO: 169) | 9 |
| | 100.00% | | AYTSSMCNPRK (SEQ ID NO: 206) | 1 |
| | 100.00% | | DHSPINLLVGVTMAHELGHNLGMNHDGDK (SEQ ID NO: 170) | 17 |
| | 100.00% | | GASLCIMRPGLTPGR (SEQ ID NO: 171) | 30 |
| | 100.00% | | KRVHELVNTINGFYR (SEQ ID NO: 173) | 3 |
| | 100.00% | | KSHDHAQLLTAINFEGK (SEQ ID NO: 174) | 7 |
| | 100.00% | | KSVGIVKDHSPINLLVGVTMAHELGHNLGMNHDGDK (SEQ | 0 |
| | | | ID NO: 207) | |
| | 100.00% | | NTLNSFGEWR (SEQ ID NO: 175) | 12 |
| | 100.00% | | RKSHDHAQLLTAINFEGK (SEQ ID NO: 208) | 3 |
| | 100.00% | | RVHELVNTINGFYR (SEQ ID NO: 177) | 4 |
| | 100.00% | | SHDHAQLLTAINFEGK (SEQ ID NO: 178) | 12 |
| | 100.00% | | SLNIDVSLTDLEIWSDQDFITVQSSAK (SEQ ID NO: 181) | 1 |
| | 100.00% | | SVGIVKDHSPINLLVGVTMAHELGHNLGMNHDGDK (SEQ ID | 3 |
| | | | NO: 182) | |
| | 100.00% | | SYEFSDDSMGYYQSFLNQYKPQCILNKPLR (SEQ ID NO: 209) | 1 |
| | 100.00% | | VFMKYNSDLNIIR (SEQ ID NO: 183) | 6 |
| | 100.00% | | VHELVNTINGFYR (SEQ ID NO: 184) | 13 |
| | 100.00% | | YNSDLNIIR (SEQ ID NO: 186) | 13 |
| | 100.00% | | YNSDLNIIRK (SEQ ID NO: 187) | 1 |

S. Data. 4: (A) FASTA sequence of FETUA sequences used to construct phylogeny in snakes.

| Protein | Species | Source | Accession | Sequence |
|---|---|---|---|---|
| A.car_FETUA | Anolis | CDNA | XP_003225747.1 | MKSLIALVLLGQILASKATSEPFPPLVSSLPDCDDPESEA |
| | carolinensis | | | AANFAVDHINSHSLHGYKYTLSRIENVKVLPRRPTGKI |
| | | | | YLLELDLAETKCHVLSPTPIQNCSVRAKVEHAVEGDCD |
| | | | | VKMLHLDGQYKVLSTKCHSSPDSREDFEKVCPDCAPL |
| | | | | ALLDDVNVVNAVNTALAQYNTYNSTDHHFELLEIARG |
| | | | | RNSHMPPGTFVEFAIAATNCTEQEAKEHKDCHVMTGE |
| | | | | HAQFGFCKATIFKKPSGEGPALFAIFPNVVICDIFDKQV |
| | | | | GHSHTHLTKHHLGKKIPSPGAGYSVLDLIHSHNNTLAS |
| | | | | HESHSAEVPVVQASPVVKRAAEVPPVIQPALPHLQQCP |
| | | | | GKYRHFDI* (SEQ ID NO: 210) |
| B.con_FETUA | Boa | CDNA | http://dx.doi. | MNSLIALVLLGQILGCTLSHHLGLSLDCNSKEAEQYAE |
| | constrictor | | org/10.5524/ | QAVHHINAHNLHGYKQILNIIEDLNIVHRRPRGEVIFIE |
| | | | 100060 | VNLLETKCHVLDPTPIENCTVRTQQEHAVETDCDVKLL |
| | | | | SDEGITKVVAAKCHSSADSVEDPMKNCAHCSVLMPLN |
| | | | | DPHVIEVVEYVLHKHNEQNPGNVYSVLEISRGQHRHE |
| | | | | PESFYVEFAIVETNCSGQDAAEGHNGCHAKAAADAHV |
| | | | | GFCRATVFRKHTATEKLTDEKFESDCVIFEPKAGESHTH |
| | | | | LIEHHFGKNIPSPGHNHTILDLIHSHNDTNASHESHSSE |
| | | | | ALPEVAAPVVKREVPETVIPPINQPTLPVKLCPGKVHHF |
| | | | | KV* (SEQ ID NO: 211) |
| P.biv_FETUA | Python | CDNA | XP_007434930.1 | MNSLIALVLLGQILGCTLSHHLLPQSDCNSEEAEHYAE |
| | bivitattus | | | EGVHYINTHNLHGYKQTLNIIKDLHVLPRRPHGKVIFT |
| | | | | ELNLLETRCHVLDPTPVENCTVRTQEEHAVEADCDVK |
| | | | | LLSDEGVTKVVAVKCHSSPDSVEDVKKLGPNYPILLPL |
| | | | | NDPRVVEVVEYVLHKHNEQQPDHVFEVLEITRGQHKH |
| | | | | EPESYYVEFVFVETNCSEQESHDAHHDCHPKAAADAH |
| | | | | VGFCKATVFRAHDATGKLTDEKFESDCVLFDQKVGET |
| | | | | HVHLIEHHLGKNIPSPGHGHTVLDLAHSHNDTNASHES |
| | | | | HSAEVLPEVAAPVVKRDVSPTLPPDDHLTPPVKLCPGK |
| | | | | VHHFKV* (SEQ ID NO: 212) |
| P.obs_FETUA | Pantherophis | genomic | WJSR01000052.1 | MNSLVALVLLGQIIGCTFSHHLLPQLDCNSEEAERLAKL |
| | obsoletus | DNA | | AVDYINEHTLHGYRQALNIIKDAHVVPRRPSGKIIFLEL |
| | | | | SLLETVCHVLDPSPLEKCTVRPQKYHAVEGDCDVKILS |
| | | | | DEGIEKVVAVKCHSEPDSAEDVLFNCPICPILLPRNDPH |
| | | | | VVESVEYVLHKHNEQLTGHSYEVLEISRGQHKLEPEAY |
| | | | | YVEFAIVETNCSAKEAHDNHHDCHPKAAGEGHLGFCR |
| | | | | ATVFRSQAATEKPKDEKYESDCIIFDVKVGIPHAHLIEH |
| | | | | HYAKNIVSPGHNNTVLDLVHSHNHTSASHETHSHEHA |
| | | | | ATVAAPVVKREAPTEGPHDHTHATNLCPGKVHHFKI* |
| | | | | (SEQ ID NO: 213) |
| T.ele_FETUA- | Thamnophis | genomic | GCA_009769535.1 | MNSLVALVLLGQIIGCTLSHHLVPQFDCNGEEAEKLGN |
| 1 | elegans | DNA | | LAVSYINEHSLHGYKHVVNQIKDAHVLPRRPHGKVIFL |
| | | | | ELDLLETVCHVLDPTPIENCSVRAQQYHAVEADCNVK |
| | | | | LVSDEGVDKVVAAKCHSEPDSVEDVRQHCPKCPILLSL |
| | | | | SDPHVINSANYVLHKHNDKLPHHAYEVLEISRGQQKF |
| | | | | DPEGYYVEFAIVETNCTHQEAHDDNYHCDPKALGEAH |
| | | | | FGLCRATVFRSHAATGKPTDEQYESDCVIFDVKDGHSH |
| | | | | SHLVEHHFRKNIVSPGYNNTVLSLIHSHNHTSASHETH |
| | | | | SHEHGTAIPPHVAKREVPTVPSHDHTHPIKLCPGKVHH |
| | | | | FKV* (SEQ ID NO: 214) |
| T.ele_FETUA- | Thamnophis | genomic | GCA_009769535.1 | MNSLVALVLLGQIIGCTLSQHLEGWQFDCHDEEVEKLA |
| 2 | elegans | DNA | | RVAVSYINEHSKHGYKQVVNQIKDAYALPQRPHGKINH |
| | | | | LELNLLETVCHVLDPTPAENCSVRAQNHHAVEADCHV |
| | | | | KLVSDEGVDKVVAAKCHSQPDSVEDVRQHCPKCQILL |
| | | | | SLSDPNVIDSANYVLHKHNDKLTHHAYEILEISRGQQK |
| | | | | FDPEGYYVEFPIVETNCTHQEAHDDTHHCHPKALGEA |
| | | | | HFGFCRATVFRSHAATGDLTDEQYESDCVIFNVKDGHS |
| | | | | HSHLVEHHFRKSIVSPGYNNTVLDLVHSHNHTSASHET |
| | | | | NSDEYGTAIPPHAGKREVPTVPSHGHALIKLFCPGEIHH |
| | | | | FKV* (SEQ ID NO: 215) |
| T.ele_FETUA- | Thamnophis | genomic | GCA_009769535.1 | MNSLVALVLLGQIIGCTLSHHFGPQFDCNGEEAERFAS |
| 3 | elegans | DNA | | VAVSYINEHSIHGYKHALNIIKDVHVLPRRPHGKVIFLE |
| | | | | LDLLETVCHVLDPTPIENCSVRAQQYHAVEADCDVKL |
| | | | | LSDEGVDKVVAAKCHSEPDSVEDVRQHCPKCPILLSLS |
| | | | | DPHVVDSANYVLHKHNDKLPHHAYEVLEISRGQQKFD |
| | | | | PEGYYVEFAIVETNCTHQEAHDDNYHCDPKALGEAHF |
| | | | | GLCRATVFRSHAATGKPTDEKYESDCVIFDVKDGHSHS |
| | | | | HLVEHHFRKSIVSPGYNNTVLDLVHSHNHTSASHETHS |
| | | | | HEHGTAIPPHVAKREVPTVPSHDHTHPIKLCPGKVHHF |
| | | | | KV* (SEQ ID NO: 216) |
| N.naj_FETUA- | Naja naja | genomic | GCA_009733165.1 | MNSLVTLVLLGQIIGCTFSHHLLPQLDCNGEEAENLAE |
| 5 | | DNA | | LAEQYINEHNLHGYKHTLNVIKDVHVLPRRPHGKVIFL |
| | | | | NLEFLETVCHVSDPTPIENCTVRPDYYHAVEAECDVRL |
| | | | | LSDEDVVKVVASKCHSEADSVENVRQKCPKCPILIPLN |
| | | | | DPHVVQSVEYVLRKYNEEHPEHVFEVLEISRGQHKYD |
| | | | | PEAFYVEFAIVETNCSAQEAHDDHHDCHPKAAGEARA |
| | | | | GFCRATVFRSHADLEKPKDELYESDCVIFDVKVGHSHT |
| | | | | HLIEHHFGKNIPSPGHNNTILDLVHSHNHTSASHESNSH |
| | | | | EHVAATEVVAPVAKREAPTAVSHDHTHATKLCPGRVH |
| | | | | HFKV* (SEQ ID NO: 217) |
| N.naj_FETUA- | Naja naja | genomic | GCA_009733165.1 | MNSLVTLVLLGQIIGCTFSVSLLRKFDCNGEEAEELAK |
| 4 | | DNA | | VAVKFINEHNLHGYKQTLNIIKEIDVRAPRPKPNVEKV |
| | | | | VEIYLNLLETKCHVMDPTPVEKCTVRQQHEHAVEADC |
| | | | | LVRIHRELGNKVTGAVCYSKPGSVLKVRQNCTKCPILI |
| | | | | PLNDPHVVQSVEYVLRKHNEQLPNHAYEVLEISRGQH |
| | | | | QYDPEAFYVEFAIVETNCSAQEAHDDHHDCHPKAAGE |
| | | | | AHTGFCRATVFRSHAALEKPKDEKYESDCVIFDVKEG |
| | | | | HSHSHLIEHHYGKNIASPGHNNTILDLVHSHNDTDTSN |
| | | | | ESDSLEAVLDPELLVKTEAPAEIAHDPTKAPPVCPGRVH |
| | | | | HFEV* (SEQ ID NO: 218) |
| N.naj_FETUA- | Naja naja | genomic | GCA_009733165.1 | MNFLVTLVLLGQIIGCTFGTTLSQEFDCNGEEAEKLAK |
| 3 | | DNA | | LAVKYINDHNLHGYKQTLNVIKEVDFPAPRPREIVEMV |
| | | | | AEMTLNVLETKCHVLDPTPVENCTVRQQHEHAVEAEC |
| | | | | VVRIHRELGDKVSGANCHSKPGSVGNVRRNCTKCPILI |
| | | | | PLNDPHVVQSVEYVLRKHNEQLPNHVYKVLEISRGQH |
| | | | | QYDPEAFYVEFAIVETNCSAQEAHDDHHVCHPKAAGE |
| | | | | GHTGFCRATVFRSHAALEKPKDEKYESDCVIFDVKKG |
| | | | | HSHSHLIDHHYGKNIASPGQNNTVLDLVHSHNHKSSSH |
| | | | | ESLSVEVMAVPLPVVKREAPAEIAHAHAKAAPLCPGK |
| | | | | VHHFKL* (SEQ ID NO: 219) |
| N.naj_FETUA- | Naja naja | genomic | GCA_009733165.1 | MNFLVTLVLLGQIIGCTFGTTLSQEFDCNGEEAEKLAK |
| 2 | | DNA | | LAVKYINDHNLHGYKQTLNVIKEVDFPAPRPREIVEMV |
| | | | | AEMTLNVLETKCHVMDPTPVENCTVRQQHEHAVEAE |
| | | | | CVVRIHRELGDKVSGANCHSKPGSVGNVRRNCMKCPI |
| | | | | LILLNDPHVLQSVEYVLRKHNEQLPNHVYKVLEISRGQ |
| | | | | HQYDPEAFYVEFAIVETNCSAQEAHDGHHVCHPKAAG |
| | | | | EGRAGFCRTTVFRSHAALEKPKDEQYELDCVIFDDKK |
| | | | | GHSHSHLIDHHYGKNIASPGHNNTVLDLVHSHNHKSSS |
| | | | | HESLSLEAMTVPLPVVKREAPAEIAHAHANAAPLCPG |
| | | | | KVHHFKL* (SEQ ID NO: 220) |
| N.naj_FETUA- | Naja naja | genomic | GCA_009733165.1 | MNFLVTLVLLGQIIGCTFGTTLSQEFDCNGEEAEKLAK |
| 1 | | DNA | | LAVKYINDHNLHGYKQTLNVIKEVDFPAPRPREIVEMV |
| | | | | AEMTLNVLETKCHVLDPTPVENCTVRQQHEHAVEAEC |
| | | | | VVRIHRELGDKVSGANCHSKPGSVGNVRRNCTKCPILI |
| | | | | PLNDPHVVQSVEYVLRKHNEQLPNHVYKVLEISRGQH |
| | | | | QYDPEAFYVEFAIVETNCSAQEAHDDHHVCHPKAAGE |
| | | | | AHTGFCRATVFRSHAALEKPKDEKYESDCVIFDVKKG |
| | | | | HSHSHLIDHHYGKNIASPGQNNTVLDLVHSHNHKSSSH |
| | | | | ESLSVEVMAVPLPVVKREAPAEIAHAHAKAAPLCPGK |
| | | | | VHHFKL* (SEQ ID NO: 221) |
| H.cur_FETUA- | Hydrophis | genomic | GCA_019472885.1 | MNSLVTLVLLCQIIGCMFSHHLSPQLDCNGEEAEKLAE |
| 1 | curtus | DNA | | LAVHYINKHNLHGYKQALNVIKDVHVLPRRPSGKVIFL |
| | | | | ELDLLETECHVLDPTPVENCTVRPQHYHAVKGDCDVK |
| | | | | LLSDEGINKVVASKCHSDPDSVEDVRQNCLNCPILIPLN |
| | | | | DPHVVQSVEYVLHKHNEKQPSHAYEVLEISRGQHKYE |
| | | | | PEAFYVEFAIVETNCSAQEAHDDHHDCHPKAAGEAHI |
| | | | | GFCRATVFRSHATLEKPKDEQYESDCVIFDVKEGHSHT |
| | | | | HLIEHHFGRNIASPGENNTVLDLIHSHNHTSASHESHSH |
| | | | | EHMVIPVVAAPVAKREAPTEISHDHTHPPKLCPGKVHH |
| | | | | FKV* (SEQ ID NO: 222) |
| H.cur_FETUA- | Hydrophis | genomic | GCA_019472885.1 | MNSLVTLVLLAQIIECMFSVVLLKESDYNGEEAKKYG |
| 2 | curtus | DNA | | DLAVHYINQHNLHGYKQTLNVIKRVDFLPPPYVEKVV |
| | | | | SVELDVLETECHVLDPTPVENCTVRQQDHHAVKSECV |
| | | | | VRIHRENGDKVFGANCHSKPDSVENVRQNCSKSPILVP |
| | | | | LNDPHVVQSVEYVLHKHNEKQPSHA*EVLEISRGQHQ |
| | | | | YEPEAFYVEFAIVETNCSVQEAHDDHHDCHPKAAGEA |
| | | | | HIGFCRATVFRSHAALEKPKDEQYESDCVILDVKEGHS |
| | | | | HTHLIEHHYGKNIPSPGHNSVVLDLAHSHNHTSSSHES |
| | | | | HSQERVVEVIVLPLPVAKREAPTETAHDHAKAAPLCPG |
| | | | | KVHHFKL* (SEQ ID NO: 223) |
| H.cur_FETUA- | Hydrophis | genomic | GCA_019472885.1 | MNSLVTLVLLCQIIGCTLSLLLRKEFDCNGEEAKKFGD |
| 3 | curtus | DNA | | LAVHYINQHNLHGYKQTLNVIKRVDFLATRS*PYVEKV |
| | | | | VSVELDVLETECHVLDPTLSRIVPVRQQDHHAVKSECV |
| | | | | VRIHRENGDKVFGANCHSKPDSVENVRQNCSKSLILVP |
| | | | | LNDPHVVQSVEYVLHKHNEKQPSHA*EVLEISRGQHQ |
| | | | | DEPEAFYVEFAIVETNCSAQEAHDDHHDCHPKAAGEA |
| | | | | HIGFCRATVFRSHAALEKPKDEQYESDCVIFDVKEGHS |
| | | | | HTHLIEHHYGKNIPSPGHNSVVLDLAHSHNHTSSSHES |
| | | | | HSQERVVEVIVLPLPVAKREAPTETAHDHAKAAPLCPG |
| | | | | KVHHFKL* (SEQ ID NO: 224) |
| V.ber_FETUA- | Vipera berus | genomic | KN624939 | MNSLIALLLLGQIIGCTFSHHLPSHGDCNGEDAKKWAH |
| 1 | | DNA | | LAVHYINEHTLHGYKQDLNIIKDIHVLPRRPRGKIIFLEL |
| | | | | ELLETVCHVLDPTPLENCTVRPQHYHAVEGDCDVKIIH |
| | | | | DEDVDKVVAAKCHSSPDSVEDVRRNCPKCPILFPLNDP |
| | | | | HVVDAIEYVLNKHNEKLSGHVYEVLEISRGQYTFEPE |
| | | | | GFYVEFAIVESNCTAQEAQDDHHHCHPNRAGEEHIGFC |
| | | | | RATVVRSHASLEKLKDEQFESDCVIFDVKEGHSYSHLI |
| | | | | EHHIGKYSTSPGHNNTVLNLAHSHNHTSASHESHSHE |
| | | | | HVVEVPVAVAKREIPTNIPPHHTHPVNLCPGKVHHFKV* |
| | | | | (SEQ ID NO: 225) |
| V.ber_FETUA- | Vipera berus | genomic | KN624939 | MNSLVALLLLSQIIGSTLSSQVRGDIDCNDKDAKDWAG |
| 2 | | DNA | | KAVRYINEHKLHGYKQDLNVIKNIHILPWDGDLVAIYL |
| | | | | KLNFLETECHVLDPTPVENCTVRPQHNHAVKIDCDVKI |
| | | | | IYDIETHKDDVFVKCNSTPDSVENVQQNCPKCPILLPQ |
| | | | | NDPHVVDAVEYVLNKHNEKLSGHVYEVLEISRGQHTF |
| | | | | EPEGFYVEFAIVESNCTAQEAQDDHHHCHPNTEGEDHI |
| | | | | GFCRATVFRSHASLEKPKDEQFESDCVIFDVKKGHAHC |
| | | | | YLIEQHVGKDSTSPGDNTNVLNLAHSHNHTRASHESH |
| | | | | SDECVVGCPVAFVKKEVPTDISDHDTPSVKGCPGRVLH |
| | | | | FNL* (SEQ ID NO: 226) |
| V.ber_FETUA- | Vipera berus | genomic | KN624939 | MNSLVALLLLGQVIGSTLSSQVRGDIDCNDKDAKHWA |
| 3 | | DNA | | DIAVRYINEHKLHGYKQDLNVIKNIHILTWDRDLVAGY |
| | | | | LKLNLLETECHVLDPTPVEKCTVRPQHNHTVEMDCDV |
| | | | | KIIYNVMTLKDEVSVKCNSTTDSVENVQRNCPKCPILL |
| | | | | PRNDPHVVDAVEYVLNKHNEKLSGHVYEVLEISRGQH |
| | | | | TFEPEGFYVEFAIVQSNCTAQEAQDDHHHCHPNTAGE |
| | | | | DHIGFCRATVFRSHASLEKPKDEQFESDCVMFDVKEG |
| | | | | HTHSYLIEQHVGKDSTSPGDNTTVLNLAHSHNHTRAS |
| | | | | HEFHSDECVVECPVAFVKKEVPTDISDHHTPPAKGCPG |
| | | | | RVLHFKL* (SEQ ID NO: 227) |
| D.acu_FETUA- | Deinag- | genomic | http://dx.doi. | MNSLVALLLLGQIIGCTFSHHLTSQVDCNGEDAEKWG |
| 1 | kistrodon | DNA | org/10.5524/ | DMAVHYINEHNLHGYKQALNVIKEIHVLPRRPHGEIVF |
| | actus | | 100196 | IEIEVLETQCHVLDETPVENCTVRPQHYHAVGGDCDV |
| | | | | KIIHHEGVDKVVGAKCHSDPDSVEDVRRNCPNCPILLP |
| | | | | LSDPRVVDCVEYVLSKHNEKLSGHIYEVLEISRGQHQY |
| | | | | EPEAFYVEFVIVEVNCTAQEAHDDHHHCHPNTAGEDH |
| | | | | IAFCRATVERSLASLEKPKDEKFESDCVILNVKEGHAH |
| | | | | SHLIQHHVGKYSISPGHNNTVLNLAHSHNHTSASHESH |
| | | | | SHEHVAEVPVAVAKREVPTDIPHHHMHSHPVKLCPGK |
| | | | | VHHFKL* (SEQ ID NO: 228) |
| D.acu_FETUA- | Deinag- | genomic | http://dx.doi. | MNSLVALLLLGQIIGSTLSSQVRGDLECDNKDAKEWA |
| 2 | kistrodon | DNA | org/10.5524/ | DVAVRYINEHKLHGYKHALNVIKNIVVVPWDGDLVAV |
| | actus | | 100196 | FLKLNLLDTECHVSDSTPVENCTVREQHNHAVEMDCD |
| | | | | VKIMFDIETFKRDVFVKCHSTPDSVENVRRDCPKCLIL |
| | | | | LPPNDPRVVDSVEYVLSKHNEQLSGHIYEVLEILRGQY |
| | | | | KYEPEAFYVEFVIVEVNCTAQEAHDDHHHCHPNTAGE |
| | | | | DHIAFCRATVFRSHVGLEKPKDKKFESDCVILHVKDGH |
| | | | | AHSHLIQQHIEKNSISPEHNITILNFIHPQNHTSTSHES |
| | | | | HEHAAEVPVDFVKKELPTDISNRHTPPVKGCPGKVHHFE |
| | | | | L* (SEQ ID NO: 229) |
| D.acu_FETUA- | Deinag- | genomic | http://dx.doi. | MNSLVALLLLGQIIGSTLSYLVKEEDLNCNSKAAKYRA |
| 4 | kistrodon | DNA | org/10.5524/ | DQVVRYINDHKLHGYKQALNVIKIFHLLSPERFSTVFY |
| | actus | | 100196 | LKLNLLDTECHVLDQTPVEKCTVRPQHNHAVEMDCD |
| | | | | AKIIFNYDIYKDYIFVKCNSTP*YEPEAFYVEFVIVEVN |
| | | | | CTAQEAHDDHHHCHPNTAGEDHIAFCRATVFRSHASL |
| | | | | EKPKDEKFESDCVILNVKRGHAYSQLIQHHVEKNSISP |
| | | | | GHNSTVLNLAHSHNHTRASHESHSDECVVECPVAFVK |
| | | | | KNIQTNIPHHTHPVEGCPGRVFHFQL* (SEQ ID NO: |
| | | | | 230) |
| D.acu_FETUA- | Deinag- | genomic | http://dx.doi. | MNSLVVLVLLGQVIGSTHSSMVKEEDLNCNSKAAKYR |
| 5 | kistrodon | DNA | org/10.5524/ | ADQAVDYINEHNLHGYKQALNVIKIIRLLPSDGLSVIFN |
| | actus | | 100196 | LKLNLLETKCHVLDPTPVENCTVRPQLNHAVETDCDV |
| | | | | KIIYNIATFKDEFLVKCHSTPGSVENILRDCPKCPLLLPL |
| | | | | SDPHVVDSVEYVLSKHNEKLSGHIYEVLEISRGQHQ*H |
| | | | | IAFCRATVFRSHASLEKPKDEKFESDCVILHVKGGHAH |
| | | | | SHLIQHHTGKYSTSPGHNSTILNVAHSHNYTSASHESHS |
| | | | | HEHVAEVPVDSVKKEVPTDIPHHHTPPVDGCPGKVLH |
| | | | | FKL* (SEQ ID NO: 231) |
| P.fla_FETUA- | Proto- | genomic | BFFQ01000665 | MNSLVALLLLGQIIGCTFSHHLPSHVPVDCNGEDAEKW |
| 1 | bothrops | DNA | | ADLAVDYINEHNLHGYKYAVNVINEIFVLPRRPHGKMI |
| | flavoviridis | | | LLELNLLETECHVLDQTPIKNCTVRPPHYHAVEGDCDV |
| | | | | KIIHDEDVDKVVAAKCYSNPDSVEDVQQNCPKCPILLS |
| | | | | LTDPHVVDSVEYVLEKHNAKLSGHIYEVLEISRGQHK |
| | | | | YEPEAYYLEFVIVEVNCTAQEAHDDHHQCHPYTAGEE |
| | | | | HIGFCRATVFRSHASLEKPKDEKFESDCVIFDIKEGHAH |
| | | | | SHLIEHHVGKYSTSPGYNNTVLNLVHSHNHTSASHESH |
| | | | | SHEHVAEVPVAVAKREVPTDIPHDHTHPVKLCPGKVHH |
| | | | | FKL* (SEQ ID NO: 232) |
| P.fla_FETUA- | Proto- | genomic | BFFQ01000665 | MNSLVALVLLGQIIGSTLSSQVRGDLECDDKEAKNWA |
| 2/HSF | bothrops | DNA | | DDAVRYINEHKLHGHKQALNVIKNICVVPWNGDLVAV |
| | flavoviridis | | | FLELNLLETECHVLDPTPVEKCTVRQQHNHAVEMDCD |
| | | | | AKIMFNVETFKRDVFVKCHSTPDSVENVRRNCSKCPIL |
| | | | | LPPNNPHVVDSVEYVLNKHNEKLSGHIYEVLEISRGQH |
| | | | | KYEPEAYYLEFVIVEINCTAQEAHDDHHQCHPYTAGED |
| | | | | HIAFCRSTVFRSHASLEKPKDEKFESDCVILDVKDGHA |
| | | | | HSHLIQQHIEKNSISPEHNITILNFVHPDNHTSTSHESHE |
| | | | | HVAEVPVVFVKKELPTDISDHHTTPVKGCPGKVHHFEL* |
| | | | | (SEQ ID NO: 3) |
| P.fla_FETUA- | Proto- | genomic | BFFQ01000665 | MNSLVALVLLGQIIGSTVSFQLGPNMDCNTKGTKDWA |
| 3/HLP | bothrops | DNA | | DIGVHYINEHKLHGYKQALNVIKIFRLLPSDGRSVIFHF |
| | flavoviridis | | | NLNLLETECHVLDSTPVENCTVRPQHNHAVEMDCNVR |
| | | | | IIHDITTFEDEVFVKCSSTPGSVENILRDCPKCPILLSPN |
| | | | | DPHVVDSVEYVLNKHNEKLSGHIYEVLEISRGQHKYEPE |
| | | | | AYYLEFVIVEINCTAQEAHDDYHQCHPYTAGEDHIAFC |
| | | | | RSTVFRSHASLEKPKDEKFESDCVILDVKEGHSHSHLIE |
| | | | | HHVGKYSTSPGYNSTDECVVECPVAFVNKEVPTDISDH |
| | | | | NTPPVKGCPGRVLHFQL* (SEQ ID NO: 233) |
| P.fla_FETUA- | Proto- | genomic | BFFQ01000665 | MNSLVALVLLGQIIGSTHSYMVKEEDLNCNSKGAKY* |
| 5 | bothrops | DNA | | ADQAVRYINEHNLHGYKQALNVIKIFRLLPSAGFSVIIY |
| | flavoviridis | | | LKLNLLETECHVLDSTPVENCTVRQQHNHAVETDCDV |
| | | | | KIIYNIMTFKDEFLVKCHSTPGSVENILRDCPKCPILLSL |
| | | | | SDPHVVDSVEYVLEKHNAKLPGHIYEVFEISRGQHKY |
| | | | | EPEALYLEFVIVEVNCTAQEAHDDHHQCHPYTAGEDHI |
| | | | | AFCRSTVFRSHASLEKPKDEKFESDCVILDVKEGHAHS |
| | | | | HLIQQHIEKNSISPEHNITILNFVHPHNHTSTSHESHEHV |
| | | | | ADVPVAFVKKELPTDISDHHTTPVKGCPGKVHHFEL* |
| | | | | (SEQ ID NO: 234) |
| G.blo_MSF | Gloydius | CDNA | Q5KQS4 | MHFLVALVLLGQIIGSTLSSQVRGDLECNDREAKEWAD |
| | blomhoffi | | | QAVRYINEHKLHEYKQALNVIKNIVVVPWNGDLVAVF |
| | | | | LKLNLLETECHVLDPTPVEKCTIRPQQNHAVEMDCDA |
| | | | | KIMFDVETFKQDVFVKCHSTPDSVEDVRRNCLKCPILL |
| | | | | SPSDPHVVDSVEYVLNKHNEQLSGHVYEVLEISRGQH |
| | | | | KYEPEAFYVEFAIVEVNCTAQEAHDDHHHCHPNTAGE |
| | | | | DHIAFCKATVFRSHASLEKPKHENFESDCVILDVKEGH |
| | | | | AHSHLIEHHIGKYSTSPGQNSTVECVAECPVAFVNKEV |
| | | | | PTDISDRHTTPVKGCPGKILHFQL* (SEQ ID NO: 2) |
| G.blo_HLP-B | Gloydius | CDNA | BAD88538 | MNSLVALVLLGQMIGSTLSHHLQSHVDCNGEDAEKWA |
| | blomhoffi | | | DMAVHYINEHNLHGYKQVFNVINEIHVLPRRPRGKIIIL |
| | | | | ELKLLETECHVLDPTPVENCTVRPPHYHAVEGDCDVKI |
| | | | | LHDEGVDKVIGAKCHSDPDSVEDVRRNCPKCPILLPLS |
| | | | | DPHVVDSVEYVLNKHNEKLSGHVYEVLEISRGQHKYE |
| | | | | PEAFYVEFAIVEVNCTAQEAHDDHHHCHPNTAGENHI |
| | | | | GFCRATVFRSHASLEKPKDEQFESDCVIFDVKEGHAHS |
| | | | | HLIEHHIGNYNTSPGHNNTVLNLAHSHNHTSASHESHS |
| | | | | HEHVAEVPVAVAKREVPTNTPHDHTHPVKLCPGKVHH |
| | | | | FKL* (SEQ ID NO: 235) |
| G.blo_HLP-A | Gloydius | CDNA | BAD88537 | MNSLVALVLLGQIIGSTLSFQLGPNMDCNTKGTKDWA |
| | blomhoffi | | | DIGVRYINEHKLDGYKNALNIIKIFRLLPSDGRSVIVHF |
| | | | | KLNLLETKCHVLDPTPVENCAVRQQHNHAVEMDCNV |
| | | | | RIIHDIATFEDEVFVKCKSTPDSVENVRRNCPKCPILLPP |
| | | | | NDPHVVDSVEYVLNKHNEKLSGHVYEVLEISRGQHK |
| | | | | YEPEAFYVEFAIVEVNCTAQEARDGHHQCHPYTAGED |
| | | | | HIAFCRATVFRSHASLEKPKDENFESDCVILDVKEGHA |
| | | | | HSHLIQQHIEKYSTSPGHNSTDEYVVECPVAFVEKEVP |
| | | | | TDMSDHDTPPVKGCPGRVLHFQL* (SEQ ID NO: 236) |
| B.jar_FETUA- | Bothrops | genomic | JAGTXL010000047.1 | MNSLVALVLLGQIIGSTLSHHLQSHVDCNGEDAEKWA |
| 1 | jararaca | DNA | | HMAVHYINEHNQHGYKCALNVINEIRLLPRRPHGTIVF |
| | | | | LKLKVLETECHVLDPTPTENCTVRPQHYHAVGGDCDV |
| | | | | KIIHEEGGDKVIGAKCYSDPDSVEDVRRNCPKCPILLN |
| | | | | LNDPQVVDSVEYVLNKHNEKVSGHVYEVLEISRGQH |
| | | | | KNEPEAYYVEFAIVEVNCTAQEAHDDHHQCHPNTAGE |
| | | | | NHIGFCRATVFRSHASLEKPKDEQFESDCVIFDVKDGH |
| | | | | AHSHLIEHHVGKYSTSPGHNNTVLNLVHSHNHTSASH |
| | | | | ESHSHEHVTEVPVAVAKREVPKDVPHDHTHPVKLCPG |
| | | | | KVHHFEL* (SEQ ID NO: 237) |
| B.jar_FETUA- | Bothrops | genomic | JAGTXL010000047.1 | MNSLVALVLLGQIIGSTLSSQVRGDLECDEKDAKEWTD |
| 2/BJ46a | jararaca | DNA | | TGVRYINEHKLHGYKYALNVIKNIVVVPWDGDWVAVF |
| | | | | LKLNLLETECHVLDPTPVKNCTVRPQHNHAVEMDCDV |
| | | | | KIMFNVDTFKEDVFAKCHSTPDSVENVRRNCPKCPILL |
| | | | | PSNNPQVVDSVEYVLNKHNEKLSDHVYEVLEISRGQH |
| | | | | KYEPEAYYVEFAIVEVNCTAQELHDDHHHCHPNTAGE |
| | | | | DHIAFCRATVFRSHASLEKPKDEQFESDCVILHVKEGH |
| | | | | AHSHLIQQHVEKDSISPEHNNTALNFVHPHNDTSTSHE |
| | | | | SHEHLAEVPVAFVKKELPKDISDRHTTPVKGCPGKVH |
| | | | | HFEL* (SEQ ID NO: 238) |
| B.jar_FETUA- | Bothrops | genomic | JAGTXL010000047.1 | MNSMVALVLLGQIIGSTLSSQVRGDLPCDDEDSKWWA |
| 3 | jararaca | DNA | | DVGVRYINEHKLHGYKYALSVIKNIVVVPWDGDWVA |
| | | | | VFLKLNLLETECHVLDPTPVKNCTVRTQHNHAVEMDC |
| | | | | DVKIMFNVETFKEDVFAKCHSTPDSVENVRRNCPKCPI |
| | | | | LLPSNNPQVVDSVEYVLNKHNEQLSDHVYEVLEISRG |
| | | | | QHKYEPEAYYVEFAIVEVNCTAQEAHDDHHQCHPNTA |
| | | | | GEDHIGFCRATVFRSHASLEKPKDEQFESDCVILHVKK |
| | | | | GHAHSHLIQQHVEKDSISPEHNNTALNFVHPHNDTRAS |
| | | | | HESHEHLAKVPVAFVKKELPKDISDRHTTPVVGCPGLR |
| | | | | AVGQPY* (SEQ ID NO: 239) |
| B.jar_FETUA | Bothrops | genomic | JAGTXL010018706.1 | MNSLVALVLLGQIIGSTHSYLVKEEDLNCNSKAAKYRA |
| | jararaca | DNA | | DQAVHYINEHKLHGYKYALNVIKIFHLLPPEEFSTVFYL |
| | | | | KLNLLETECHVFDPTPVENCNVRPQHNHAVEMDCDV |
| | | | | KIIFNFQFFKTEVFVKCNSTPDSVENVRRNSPKCSILLPP |
| | | | | NDPQVIDSVEYVLNKHNEQLSGHVYKVLEISRGQHKY |
| | | | | EPEAYYVEFAIMEINCTAQEAHDDHHQCHPNTAGEDHI |
| | | | | GFCRATVFRSHASLEKPKDEQFESDCVILDVKEGHAHS |
| | | | | HLIQQHVEKYSTSPGHNNTALNLVHLHNDTSTSHKSHS |
| | | | | GKCPVVFPKKKVPTDIPHHTHPVEGCPGKVLHFEL* |
| | | | | (SEQ ID NO: 240) |
| C.tig_FETUA- | Crotalus | genomic | XP_039207926.1 | MNSLVALVLLGQIIGCTFSHHLQSQVDCNGEDAEKWA |
| 1 | tigris | DNA | | DMAVHYINEHNLHGYKYTTNVINEIHVLPRRPHGVIVF |
| | | | | LELKVLETQCHVLDPTPVENCTVRPQHYHAVGGDCDV |
| | | | | KILHDEGVDKVIGAKCHSDPDSVEDVRQNCSKCPILLS |
| | | | | LSDPHVVDSVEYVLNKYNEKLSGHVYEVLEISRGQHK |
| | | | | YEPEAFYVEFAIVEVNCTAEEAHDDHHHCHPNTDGEN |
| | | | | HIGFCKATVFRSHASLEKPKDEMFESDCVIFDVKEGHA |
| | | | | HSHLIEHHVGKYSTSPGHNSTVLNLVHSHNHTSASHES |
| | | | | HSHEHVMEVPVAVAKREVPIDVPHDHTHPVKLCPGKV |
| | | | | HHFQL* (SEQ ID NO: 241) |
| C.tig_FETUA- | Crotalus | genomic | XP_039207927.1 | MNSLVALVLLGXSIGSTLSSQVRGDLECDNKEAKEWA |
| 2 | tigris | DNA | | DMAVRYINEHKLHGYKQALNVIKNILVVPWDGDLVAV |
| | | | | YLKLNLLETECHMLDPHPVENCTIRPQNNHAVKMDCN |
| | | | | AKIMFDVVTFKQDVFVKCHSTPDSVENVRRNCPKCPIL |
| | | | | LPWNDSHVVDSVEYVLNKHNEKLSGHVYEVLEISRGQ |
| | | | | HKYEPEAFYVEFAIVEVNCTAEEAHDDHHHCHPNTAG |
| | | | | EDHIGFCRATVFRSHASLEKPKDEKFESDCVILHVKEG |
| | | | | YAYSHLLYKQIEKYNVPPEFRNTVLNLTHSHNHTSTSH |
| | | | | ESHEHVAEVPVAFVKKELPTDISDHDTPPVEGCPGKAL |
| | | | | HFQL* (SEQ ID NO: 242) |
| C.tig_FETUA- | Crotalus | genomic | XP_039207923.1 | MNSLVALVLLGQIIGSTLSFQLAPNMDCNTKGTKDWA |
| 3 | tigris | DNA | | DIGVRYINEHKLHGYKQALNIIKIFRLLPSDGLSVMFHF |
| | | | | KLNLLETECHVLDPTPVENCIVRQKNNHAVEMDCNVR |
| | | | | IIHDIATFGDEVFVKCNSTPDSVENVRRNCPKCPILLPPN |
| | | | | DPHVVDSVEYVLNKHNEQLSGHVYEVLEISRGQHKYE |
| | | | | PEAFYVEFAIVEVNCTAEEAHDDHHHCHPNTAGEDHIG |
| | | | | FCRATVFRSHASLEKPKDEKFESDCVILHVKKGHAYSH |
| | | | | LIEHHVGKYNTSPGRNSTDECVVECPVAFVNKEVPKDI |
| | | | | SDHDTPPVEGCPGKALHFQL* (SEQ ID NO: 243) |
| C.tig_FETUA- | Crotalus | genomic | XP_039207924.1 | MNSLVVLVLLGQIIGSTHSYMVREEHLNCNSKAAKYR |
| 4 | tigris | DNA | | ADQAVRYINEHKLHGYKQALNIIKTFHLLPPERFTTVF |
| | | | | YLKLNLLETECHVLDPTPVENCTVRPQNNHAVEMDCD |
| | | | | VKINFNFEFFKTEIFVKCNSTPNSVENVRRNCPECPILLP |
| | | | | PNDPHVIDSVEYVLNKHNEKLSGHVYEVLEISRGQHK |
| | | | | YEPEAFYVEFAIVEVNCTAEEAHDDHHHCHPNTAGED |
| | | | | HIGFCRATVFRSHASLEKPKDEKFESDCVILHVKEGHA |
| | | | | TSHLIEHHTGKYSTSPGRNSTDECVVECPVAFVKKEVP |
| | | | | THIPHHTHPVEGCPGKVFHFQL* (SEQ ID NO: 244) |
| C.tig_FETUA- | Crotalus | genomic | LOC120311907 | MNSLVVLVLLGQIIGSTLSYMVREEDLNCNSKAAKYR |
| 5 | tigris | DNA | | ADQAVHYINEHNLHGYKQALNIIKTIRLLPSDGLSVIIY |
| | | | | LKLNLLETECHVLDPTPVENCTVRQQNNHAVETDCDV |
| | | | | KIIYNIETFKDEFLVKCHSTPGSVENILQDCPKCPILMPL |
| | | | | SEPHVTDSVEYVLNKHNEQLSGHVYEVLEISRGQHKY |
| | | | | EPEAFYVEFAIVEVNCTAQEAHDDHHHCHPNTDGENH |
| | | | | IGFCRATVFRSYASLEKPKVEMSESDCVILDVKEGHAH |
| | | | | SHLIEHHVGKYSISPGHNNTVLNLAHSHNHTSASHESH |
| | | | | SHEHVTEVPVAVAKREVPTDIPHHHTPSVDGCPGKVLH |
| | | | | FKL* (SEQ ID NO: 245) |
| C.hor_FETUA- | Crotalus | genomic | OP223732 | MNSLVALVLLGQIIGSTHSYMVREEDLNCNSKAAKYR |
| 5 | horridus | DNA | | ADQAVHYINEHNLHGYKQALNIIKTIRLLPSDGLSVIM |
| | | | | YLKLNLLETECHVLDPTPVENCTVRQQHNHAVETDCD |
| | | | | VKIIYNIETFKDEFLVKCHSTPGSVEKILEDCPKCPLLLS |
| | | | | LSDPHVVDSVEYVLNKHNEKLSGHVYEVLEISRGQHK |
| | | | | YEPEAFYVEFAIVEVNCTAQEAHDDHHHCHPNTAGEN |
| | | | | HIAFCRATVFRSYASLEKPKDEKFESDCVILHVKEGHT |
| | | | | HSHLIEHHIGKYSTSPGHNSTVLNLAHSHNHTSASHES |
| | | | | HSHEHVTEVPVAVAKREDPTDIPHHHTPSVDGCPGKVL |
| | | | | HFKL* (SEQ ID NO: 246) |
| C.hor_FETUA- | Crotalus | genomic | OP223732 | MNSLVVLVLLCQIIGSTYSYMVREEDLNCNSKAAKYR |
| 4 | horridus | DNA | | ADQAVRYINEHKLHGYKQALNIIKIFHLLPPERFMTVF |
| | | | | YLKLNLLETECHVLDPTPVENCTVRPQNNHAVEMDCD |
| | | | | VKINFNFEFFKTEIFVKCNSTPDSVENVQRNCPECPILLP |
| | | | | PNDPHVINSVEYVLNKHNEKLSGHVYEVLEISRGQHK |
| | | | | YEPEAFYVEFAIVEVNCTAEEAHNDHHHCHPNTAGKD |
| | | | | HIGFCRATVFRSHASLEKPKDEMFESDCVIFDVKKGHA |
| | | | | HSHLIQQHIGKYNISPGHNNAVLNLAHSHNDTRASQES |
| | | | | HSDECVVECPVAFVKKEVPTHIPHHTHPVEGCPGKVFH |
| | | | | FQL* (SEQ ID NO: 247) |
| C.hor_FETUA- | Crotalus | genomic | OP223732 | MNSLVALMLLGQIIGSTLSFQLAANMDCNTKGTKDWA |
| 3 | horridus | DNA | | DIGVRYINEHKLHGYKQDLNIIKIFRLLPSDGRSVIFHFK |
| | | | | LNLLETECHVLDPTPVENCTVRQQNNHAVEMDCNVRI |
| | | | | IHDIATFEDEVFVKCNSTPDSVENVRRNCPKCPILLPPN |
| | | | | DPHVVDSVEYVLNKHNEKLSGHIYEVLEISRGQHKYE |
| | | | | PEAFYVEFAIVEVNCTAEEAHDDHHHCHPNTDGENHI |
| | | | | GFCRSTVFRSHASLEKPKDEKFESDCVILHVKEGHAHS |
| | | | | HLIEHHVGKYSTSPGRNSTDECVVECPVAFVNKEVPKD |
| | | | | ISDHDTPPVEGCPGKVLHFQL* (SEQ ID NO: 248) |
| C.hor_FETUA- | Crotalus | genomic | OP223732 | MNSLVALVLLGQIIGSTLSSQVRGDLECDDKEAKEWAD |
| 2 | horridus | DNA | | IAVRYINEHKLHGYKQALNVIKNILVVPWNGDLVAVFL |
| | | | | KLNLLETECHVLDPTPVENCTVRPQNNHAVEMDCDAK |
| | | | | IMFDVVTFKTDVFVKCHSTPDSVENVRRNCPKCPILLP |
| | | | | RNDSHVVDSVEYVLNKHNEKLSGHVYEVLEISRGQHK |
| | | | | SEPEAFYVEFAIVEVNCTAQEAHDDHHHCHPNTTGED |
| | | | | HIGFCRATVFRSHASLEKPKDEKFESDCVIFDVKEGHA |
| | | | | HSHLIEHHVGKYSTSPGRNSTDECVVECPVAFVNKELP |
| | | | | TDISDHHTTPVKGCPGKVLHFQL* (SEQ ID NO: 249) |
| C.hor_FETUA- | Crotalus | genomic | OP223732 | MNSLVALVLLGQIIGCTFSHHLQSQVDCNGEDAEKWA |
| 1 | horridus | DNA | | DMAVHYINEHNLHGYKYTNNVINEIHVLPRRPRGEIVF |
| | | | | LELKVLETQCHVLDPTPVENCTVRPLHYHAVGGDCDV |
| | | | | KIIHDEGVDKVIGAKCHSDPDSVEEVRRNCPECPILLSL |
| | | | | SDPHVINCVEYVLNKYNEKLSGHVYEVLEISRGQHKY |
| | | | | EPEAFYVEFAIVEVNCTAEEAHDDHHHCHPNTDGENHI |
| | | | | GFCRATVFRSHASLEKPKDEMFESDCVIFDVKEGHAHS |
| | | | | HLIEHHVGKYSTSPGHNNTVLNLIHSHNHTSASHESHS |
| | | | | HEHMTEVPVAVAKREVPTDVPHDHIHPVKLCPGKVHH |
| | | | | FKL* (SEQ ID NO: 250) |
| C.scu_FETUA- | Crotalus | genomic | | MNSLVALVLLGQIIGSTHSYMVREEDLNCNSKAAKYR |
| 5 | scutulatus | DNA | | ADQAVHYINEHNLHGYKQALNIIKTIRLLPSDGLSVIIY |
| | | | | LKLNLLETECHVLDPTPVENCTVRQQNNHAVETDCDV |
| | | | | KIIYNIETFKDEFLVKCHSTPGSVEKILQDCPKCPILLSL |
| | | | | SDPHVVDSVEYVLNKHNEQLSGHVYEVLEISRGQHKYE |
| | | | | PEAFYVEFAIVEVNCTAQEAHDDHHHCHPNTAGEDHI |
| | | | | AFCRATVFRSYASLEKPKDEMSESDCVILDVKEGHAHS |
| | | | | HLIEHHTGKYSTSPGHNSTVLNLAHSHNHTSASHESHS |
| | | | | HEHVTEVPVAVAKREVPTDIPHHHTPSVDGCPGKVLHF |
| | | | | KL* (SEQ ID NO: 251) |
| C.scu_FETUA- | Crotalus | genomic | | MNSLVALVLLGQIIGSTLSYMVREEDLNCNSKAAKYR |
| 4 | scutulatus | DNA | | ADQAVRYINEHKLHGYKQALNIIKTFHLLPPERFTTVF |
| | | | | YLKLNLLETECHVLDPTPVENCTVRPQYNHAVEMDCD |
| | | | | VKINFNFEFFKTEIFVKCNSTPDSVENVR*NCPECPILLP |
| | | | | PNDPHVIDSVEYVLNKHNEKLSGHIYEVLEISRGQHKY |
| | | | | EPEAFYVEFAIVEVNCTAEEAHDDHHHCHPNTAGEDHI |
| | | | | GFCRATVFRSHASLEKPKDEKFESDCVILHVKKGHAHS |
| | | | | HLIQQHIGKYNISPGHHNTVLNLAHSHNDTRASQESHS |
| | | | | DECVVECPVAFVKKEVPTHIPHHTHPVEGCPGKVHHF |
| | | | | KL* (SEQ ID NO: 252) |
| C.scu_FETUA- | Crotalus | genomic | | DGLSVIFHFKLNLLETECHVLDPTPLENCTVRQQNNHA |
| 3 | scutulatus | DNA | | VEMDCNVRIIHDIATFEDEVFVKCNSTPDSVENVRRNC |
| | | | | PKCPILLPPNDPHVVDSVEYVLNKHNEKLSGHIYEVLEI |
| | | | | SRGQHKYEPEAFYVEFAIVEVNCTAEEAHDDHHHCHP |
| | | | | NTDGENHIGFCRATVFRSHASLEKPKDEKFESDCVILH |
| | | | | VKKGHAYSHLIEHHVGKYNTSPGRNSTDECVVECPVA |
| | | | | FVNKEVPKDISDHDTPPVEGCPGKALHFQL* (SEQ ID |
| | | | | NO: 253) |
| C.scu_FETUA- | Crotalus | genomic | | MNSLVALVLLGQIIGSTLSSQVRGDLECDDKEAKEWAD |
| 2 | scutulatus | DNA | | MAVRYINEHKLHGYKQALNVIKNILVVPWDGDLVAVY |
| | | | | LKLNLLETECHVLDPTPVENCTIRPQNNHAVKMDCNA |
| | | | | KIMFDVVTFKQDVFVKCHSTPDSVENVRRNCPECPILL |
| | | | | PRNDSHVVDSVEYVLNKHNEKLSGHVYEVLEISRGQH |
| | | | | KYEPEAFYVEFAIVEVNCTAQEAHDDHHHCHPNTAGE |
| | | | | NHIGFCRATVFRSHASLEKPKDEKFESDCVILHVKEGH |
| | | | | AHSHLIEHHVGKYNTSPGRNSTDECVVESSLLTKATGR |
| | | | | PVAFVNKEVPADISDHHTTPVKGCPGKVLHFQL* (SEQ |
| | | | | ID NO: 254) |
| C.scu_FETUA- | Crotalus | genomic | | MNSLVALVLLGQIIGCTFSHHLQSQVDCNGEDAEKWA |
| 1 | scutulatus | DNA | | DMAVHYINEHNLHGYKYTTNVINEIHVLPRRPHGEIIFL |
| | | | | ELKVLETQCHVLDPTPVENCTVRPLHYHAVGGDCDVK |
| | | | | ILHDEGVDKVIGAKCHSDPDSVEDVRRNCPNCPILLSL |
| | | | | SDPHVVDSVEYVLNKHNEKLSGHVYEVLEISRGQHKY |
| | | | | EPEAFYVEFAIVEVNCTAEEAHDDHHHCHPNTAGENHI |
| | | | | GFCRATVFRSHASLEKPKDEKFESDCVIFDVKEGHAHS |
| | | | | HLIEHHTGKYSTSPGHNNTVLNLVHSHNHTSASHESHS |
| | | | | HEHMTEVPVAVAKREVPTDVPHDHTHPVKLCPGKVHH |
| | | | | FKL* (SEQ ID NO: 255) |
| C.atx_FETUA- | Crotalus | CDNA | OP223733 | MNSLVALVLLGQIIGSTHSYMVREEDLNCNSKAAKYR |
| 5 | atrox | | | ADQAVRYINEHNLHGYKQALNIIKTIRLLPSDGLLVVIY |
| | | | | LKLNLLETECHVLDPTPVENCTVRQQNNHAVETDCDV |
| | | | | KVIYNIETFKDEFLVKCHSTPGSVEKILQDCPKCPILLSL |
| | | | | SDPHVVDSVEYVLNKHNEQLSGHVYEVLEISRGQHKY |
| | | | | EPEAFYVEFAIVEVNCTAQEAHDDHHHCHPNTAGENHI |
| | | | | AFCRATVFRSYASLEKPKDEKFESDCVILHVKEGHAHS |
| | | | | HLIEHHTGKHSTSPGHNSTVLNLAHSHNHTSASHESHS |
| | | | | HEHVMEVPVAVANREDPTDIPHHHTPSVDGCPGKVLH |
| | | | | FKL* (SEQ ID NO: 256) |
| C.atx_FETUA- | Crotalus | CDNA | OP223733 | MNSLVALVLLGQIIGSTLSYMVKEEDMNCNSKAAKYR |
| 4 | atrox | | | ADQAVRYINEHKLHGYKQALNIIKTFHLLPPERFTTVF |
| | | | | YLKLNLLETECHVLDPTPVENCTVRPQNNHAVEMDCD |
| | | | | VKINFNFEFFKTEIFVKCNSTPDSVENVQRNCPKCPILL |
| | | | | PPNDPHVINSVEYMLNKHNEKLSGHVYEVLEISRGQH |
| | | | | KYEPEAFYVEFAIVEVNCTAEEAHDDHHHCHPNTAGE |
| | | | | DHIGFCRATVFRSHASLEKPKDEMFESDCVIFDVKKGH |
| | | | | SHSHLIQQHIGKYNISPGHHNTVLNLAHSHNDTRASHE |
| | | | | SHSDECVVECPVAFVKKEVPTHIPHHTHPVEGCPGKVH |
| | | | | HFKL* (SEQ ID NO: 257) |
| C.atx_FETUA- | Crotalus | CDNA | OP223733 | MNSLVALVLLGQIIGSTLSFQLAGNMDCNTKGTKDWA |
| 3 | atrox | | | DIGVRYINEHKLHGYKQALNIIKIFRLLPSDGRSVIFHFK |
| | | | | LNLLETECHVLDPTPLENCTVRQQNNHAVEMDCNVRII |
| | | | | HDIATFEDEVFVKCNSTPDSVENVRRNCPKCPILLPPND |
| | | | | PHVIDSVEYVLNKHNEKLSGHIYEVLEISRGQHKYEPE |
| | | | | AFYVEFAIVEVNCTAEEAHDDHHDCHPNTDGENHIGF |
| | | | | CRATVFRSHASLEKPKEEMFESDCVILHVKKGHAYSHL |
| | | | | IEHHVGKYNTSPGRNSTDECVVQCPVAFVNKEVPKDIS |
| | | | | DHDTPPVEGCPGKALHFQL* (SEQ ID NO: 258) |
| C.atx_FETUA- | Crotalus | CDNA | | MNSLVALVFLGQIIGSTLSSQVRGDLECDDKEAKEWAD |
| 2 | atrox | | | MAVCYINEHKLHGYKQALNVIKNILVVPWDGDLVAVY |
| | | | | LKLNLLETECHVLDPHPVENCTIRPQNNHAVKMDCNA |
| | | | | KIMFDVVTFKQDVFVKCHSTPDSVENIRRNCPKCPILL |
| | | | | PRNDSHVVDSVEYVLNKHNEKLSGHIYEVLEISRGQH |
| | | | | KYEPEAFYVEFAIVEVNCTAQEAHDDHHHCHPNTAGE |
| | | | | DHIGFCRATVFRSHASLEKPKDEKFESDCVILHVKEGH |
| | | | | VHSHLIEHHTGKYSTSPGSNSTDECVVQCPVAFVNKEL |
| | | | | PTDISDHHTTPVKGCPGKVFHFQL* (SEQ ID |
| | | | | NO: 259) |
| C.atx_FETUA- | Crotalus | CDNA | OP223733 | MNSLVALVLLGQIIGCTFSHHLQSQIDCNGEDAEKWAD |
| 1 | atrox | | | MAVHYINEHNLHGYKYTTNVINEIHVLPWRPHGEIIFL |
| | | | | ELKVLETQCHVLDPTPVENCTVRPLHYHAVGGDCDVK |
| | | | | IIHDEGVDKVIGAKCHSDPDSVENVRQNCPECPILLSLS |
| | | | | DPHVVDCVEYVLNKYNEKLSGHIYEVLEISRGQHKYE |
| | | | | PEAFYVEFAIVEVNCTAEEAHDDHHHCHPNTAGENHIG |
| | | | | FCRATVFRSHASLEKPKDEMFESDCVIFDVKEGHAHSH |
| | | | | LIEHHTGKYSTSPGHNNTVLNLIHSHNHTSASHESHSD |
| | | | | EHMTEVPVAVAKREVPTDVPHDHTHPVKLCPGKVHHF |
| | | | | KL* (SEQ ID NO: 8) |
| C.ada_FETUA- | Crotalus | genomic | OP223730 | MNSLVALVLLGQIIGSIHSYMVREEDLNCNSKAAKYRA |
| 5 | adamanteus | DNA | | DQAVQYINEHNLHGYKQALNIIKTIRLLPSDGLLVVIYL |
| | | | | KLNLLETECHVLDPTPVENCTVRPQNNHAVETDCDVK |
| | | | | IIYNIETFKDEFLVKCHSTPGSVEKILQDCPKCPILLPPN |
| | | | | DPHVVDVEYVLNKHNEKLSGHVYEVLEISRGQHKYEP |
| | | | | EAFYVEFAIVEVNCTAQEAHDDHHHCHPNIAGEDHIGF |
| | | | | CRATVFRSYASLEKPKDEKFESDCVILDVKEGHAHSHL |
| | | | | IQQHIGKYSTSPGHNNTVLNLAHSHNHTSASHESHSHE |
| | | | | HVTEVPVAVAKREDPTDIPHHHTPSVDGCPGKVLHFKL* |
| | | | | (SEQ ID NO: 260) |
| C.ada_FETUA- | Crotalus | genomic | OP223730 | MNSLVVLVLLGQIIGSTHSYMVREEDMNCNSKAAKYR |
| 4 | adamanteus | DNA | | ADQAVRYINEHKLHGYKQALNIIKTFHLLPPERFTTVF |
| | | | | YLKLNLLETECHVLDPTPVENCTVRPQNNHAVEMDCD |
| | | | | VKINFNFEFFKTEIFVKCNSTPDSVENVRRNCPECPILLP |
| | | | | PNDPHVIDSVEYVLNKHNEKLSGHIYEVLEISRGQHKY |
| | | | | EPEAFYVEFAIVEVNCTAEEAHDDHHHCHPNTDGENHI |
| | | | | GFCRATVFRSHASLEKPKDEKFESDCVILHVKEGHAHS |
| | | | | HLIQQHIGKYNISPGHNNTVLNLAHSHNDTRASQESHS |
| | | | | DECVVECPVVFVKKEVPTHIPHHTHPVEGCPGKVFHF |
| | | | | QL* (SEQ ID NO: 261) |
| C.ada_FETUA- | Crotalus | genomic | OP223730 | MNSLVALMLLGQIIGSTLSFQLAGNMDCNTKGTKDWA |
| 3 | adamanteus | DNA | | DIGVRYINEHKLHGYKQALNIIKIFRLLPSGGLSVIFHFK |
| | | | | LNLLETECHVLDPTPVENCTVRPQNNHAVEMDCNVRII |
| | | | | HDIATFEDEVFVKCNSTPDSLENVRRNCPKCPILLPPND |
| | | | | PHVVDSVEYVLNKHNEKLSGHIYEVLEISRGQHKYEPE |
| | | | | AFYVEFAIVEVNCTAEEAHDDHHHCHPNTDGENHIGF |
| | | | | CRATVFRSHASLEKPKDEKFESDCVILHVKEGHAHSHL |
| | | | | IQHHTGKYSTSPGRNSTDECVVECPVAFVNKELPTDIS |
| | | | | DHDTPPVEGCPGKVLHFQL* (SEQ ID NO: 262) |
| C.ada_FETUA- | Crotalus | genomic | OP223730 | MNSLVALVFLGQIIGSTLSSQVRGDLECDDKEAKEWAD |
| 2 | adamanteus | DNA | | MAVRYINEHKLHGYKEALNVIKNILVVPWNGDLVAVF |
| | | | | LKLNLLETECHVLDPTPVENCTVRPQNNHAVEMDCDA |
| | | | | KIMFDVVTFKKDVFVKCLSTPDSVENVRRNCPKCPILL |
| | | | | PRNDSHVVDSVEYVLNKHNEKLSGHVYEVLEISRGQH |
| | | | | KYEPEAFYVEFAIVEVNCTAQEAHDDHHHCHPNTAGE |
| | | | | DHIGFCRATVFRSHASLEKPKDEKFESDCVILHVKEGH |
| | | | | AHSHLIEHHTGKYSTSPGRNSTDECVVQCPVAFVNKEL |
| | | | | PTDISDHHTTPVKGCPGKVFHFQL* (SEQ ID |
| | | | | NO: 263) |
| C.ada_FETUA- | Crotalus | genomic | OP223731 | MNSLVALVLLGQIIGCTFSHHLQSQIDCNGEDAEKWAD |
| 1 | adamanteus | DNA | | MAVHYINEHNLHGYKYTTNVINEIHVLPRRPHGEIIFLE |
| | | | | LKVLETQCHVLDPTPVENCTVRPLHYHAVGGDCDVKII |
| | | | | HDEGVDKVIGAKCHSDPDSVENVRQNCPECPILLSLSD |
| | | | | PHVIDSVEYVLNKYNEKLSGHVYEVLEISRGQHKYEPE |
| | | | | AFYVEFAIVEVNCTAEEAHDDHHHCHPNTDGENHIGF |
| | | | | CRATVFRSHATLEKPKDEMFELDCVIFDVKEGHAHSHL |
| | | | | IEHHVGKYSTSPGHNNTVLNLIHSHNHTSASHESHSHE |
| | | | | HMMEVPVAVAKREVPTDVPHDHTHPVKLCPGKVHHF |
| | | | | KL* (SEQ ID NO: 264) |

What is claimed is:

1. A method comprising administering to an individual who has experienced envenomation by a snake a composition comprising a protein that has an amino acid sequence that is at least 95% identical to SEQ ID NO:1 such that development of injury to the individual from the envenomation is inhibited.

2. The method of claim 1, wherein the snake is a species of the genus Crotalus, Agkistrodon, or Sistrurus.

3. The method of claim 2, wherein inhibition of the injury comprises reduced proteolysis of collagen, fibrinogen, or a combination thereof.

4. The method of claim 3, wherein the composition further comprises at least one additional anti-venom agent, wherein the at least one additional anti-venom agent is an antibody or antigen binding fragment thereof that specifically binds to a component of snake venom.

5. The method of claim 4, wherein the at least one least one additional anti-venom agent is a monoclonal antibody or antigen binding fragment thereof that specifically binds to a component of snake venom, or is a group of polyclonal antibodies or antigen binding fragments thereof, wherein the polyclonal antibodies or antigen binding fragments thereof specifically bind to a plurality of proteins in snake venom.

6. The method of claim 5, wherein the individual is a human, a canine animal, a feline animal, an equine animal, a porcine animal, or a bovine animal.

7. A composition comprising a combination of a protein that has an amino acid sequence that is at least 95% identical to SEQ ID NO:1 and at least one additional anti-venom agent.

8. The composition of claim 7, wherein the at least one additional agent is a monoclonal antibody or antigen binding fragment thereof that specifically binds to a component of snake venom.

9. The composition of claim 8, wherein the at least one additional agent comprises polyclonal antibodies or antigen binding fragments thereof that specifically bind to a plurality of proteins in snake venom.

10. The composition of claim 9, wherein the composition is more effective at inhibiting development of an injury to an individual who is envenomated by a snake, relative to the effectiveness of a composition comprising the polyclonal antibodies or antigen binding fragments thereof, but does not comprise the protein that is at least 95% identical to SEQ ID NO:1.

11. A kit comprising an isolated or recombinantly produced protein that has an amino acid sequence that is at least 95% identical to SEQ ID NO:1 and a sealed container comprising said protein, the kit optionally including printed materials instructing a user to administer the composition to an individual who has experienced a snake bite, the kit further optionally comprising a syringe.

12. The kit of claim 11, further comprising at least one antibody or antigen binding fragment thereof that specifically binds to at least one protein in venom of a snake.

13. The kit of claim 12, wherein the at least one antibody is present in a group of polyclonal antibodies or antigen binding fragments thereof, wherein the polyclonal antibodies or the fragments thereof specifically bind to a plurality of proteins in snake venom.
